SEQUENCE LISTING (5 sequences)

SEQ ID NO: 1 - protein, 95 aa, Artificial Sequence
  Description of Artificial Sequence; Note = Synthetic Construct
SDKLNEEAAKNIMVGNRCEVTVGAQMARRGEVAYVGATKFKEGVWVGVKYDEPVGKNDGSVAGVRYFDCDPKYGGFVRPVDVKVGDFPELSIDEI

SEQ ID NO: 2 - protein, 5 aa, Artificial Sequence
  Description of Artificial Sequence; Note = Synthetic Construct
GKNDG

SEQ ID NO: 3 - protein, 5 aa, Artificial Sequence
  Description of Artificial Sequence; Note = Synthetic Construct
GKHDG

SEQ ID NO: 4 - protein, 5 aa, Artificial Sequence
  Description of Artificial Sequence; Note = Synthetic Construct
GKNSG

SEQ ID NO: 5 - protein, 5 aa, Artificial Sequence
  Description of Artificial Sequence; Note = Synthetic Construct
GKHSG

(19) United States
(12) Patent Application Publication
(10) Pub. No.: US 2003/0211538 A1
(43) Pub. Date: Nov. 13, 2003

(54) CAP-Gly domain structure and uses thereof

(76) Inventor: Ming Luo, Vestavia Hills, AL (US)

Correspondence Address: NEEDLE & ROSENBERG, P.C., SUITE 1000, 999 PEACHTREE STREET, ATLANTA, GA, 30309-3915, US

(21) Appl. No.: 10/309,763

(22) Filed: Dec. 3, 2002

Related U.S. Application Data
(60) Provisional application No. 60/338,171, filed on Dec. 3, 2001.

(51) Int. Cl. G01N 33/53; G06F 19/00; G01N 33/48; G01N 33/50; C07K 14/47

(52) U.S. Cl. 435/7.1; 530/350; 702/19

(57) ABSTRACT

The present invention relates to a polypeptide that includes amino acid sequence of the CAP-Gly domain or a portion thereof and heterologous amino acid sequence, the structure of any such polypeptide and its use in designing, identifying or validating ligands to the CAP-Gly domain or homologous structure.

### CROSS-REFERENCE TO RELATED APPLICATIONS

This application claims the benefit of priority of U.S. Provisional Application No. 60/338,171, filed Dec. 3, 2001, which is hereby incorporated in its entirety herein by reference.

### STATEMENT OF GOVERNMENT SUPPORT

This invention was made with government support under National Institute of General Medical Sciences (NIGMS) Grant IP50-GM62407 and NASA Cooperative Agreement NCC8-126. The government may have certain rights in the invention.

### FIELD OF INVENTION

The disclosed invention is generally related to structure of and use of the structure of cytoskeletal associated proteins (CAPs). More particularly, the disclosed invention relates to any structure which includes a conserved motif, the CAP-Gly domain, that has been identified in a number of CAPs.

### SUMMARY OF THE INVENTION

In accordance with the purpose(s) of this invention, as embodied and broadly described herein, this invention, in one aspect, relates to a polypeptide that includes amino acid sequence of the CAP-Gly domain or a portion thereof and heterologous amino acid sequence.

In a second aspect, the present invention relates to an isolated polypeptide containing an amino acid sequence according to amino acid residues 135 to 229 of a cytoskeletal-associated protein with structure analogous to the structure of the CAP-Gly domain of F53F4.3 protein.

In a third aspect, the present invention relates to a crystal of the CAP-Gly domain, wherein the space group of the crystal is P6122 and unit cell dimensions of the crystal are: about a=64±3 Å, b=64±3 Å, and c=102±3 Å; about a=64±2 Å, b=64±2 Å, and c=102±2 Å; or about a=64±1 Å, b=64±1 Å, and c=102±1 Å.

In a fourth aspect, the present invention relates to a method of characterizing protein structures that includes the steps of: determining the three-dimensional structure of the CAP-Gly domain; determining the three-dimensional structure of an experimental protein; comparing the three-dimensional structure of the experimental protein to the three-dimensional structure of the CAP-Gly domain; and recording variances between the three-dimensional structure of the CAP-Gly domain and the experimental protein.

In a fifth aspect, the present invention relates to a method of evaluating two or more experimental proteins in respect to the CAP-Gly domain that includes the steps of: evaluating the variances between the three-dimensional structure of each experimental protein and the three-dimensional structure of the CAP-Gly domain; ranking the experimental protein with the least variance from the structure of CAP-Gly domain as being most similar to the CAP-Gly domain.

In a sixth aspect, the present invention relates to a method for generating analogs of polypeptides that contain the CAP-Gly domain that includes the steps of: determining the structure of a CAP-Gly domain; selecting a polypeptide containing an amino acid sequence that maintains a CAP-Gly domain structure; and generating an analog polypeptide containing the amino acid sequence that maintains the CAP-Gly domain structure.

In a seventh aspect, the present invention relates to a method for determining whether an analog of the CAP-Gly domain will have an altered three-dimensional structure as compared to the CAP-Gly domain. This method includes the steps of: determining the three-dimensional coordinates of atoms of a CAP-Gly domain; providing a computer having a memory means, a data input means, a visual display means, the memory means containing three-dimensional molecular simulation software operable to retrieve coordinate data from the memory means and to display a three-dimensional representation of a molecule on the visual display means and being operable to produce a three-dimensional representation of an analog of the molecule responsive to operator-selected changes to the chemical structure of the molecule and to display the three-dimensional representation of the analog; inputting three-dimensional coordinate data of the atoms of the CAP-Gly domain into the computer and storing the data in the memory means; displaying a three-dimensional representation of the CAP-Gly domain on the visual display means; inputting into the data input means of the computer at least one operator-selected change in chemical structure of the CAP-Gly domain; executing the molecular simulation software to produce a modified three-dimensional molecular representation of the analog structure; and displaying the three-dimensional representation of the analog on the visual display means, whereby changes in three-dimensional structure of the Cap-Gly domain consequent on changes in chemical structure can be visually determined.

In an eighth aspect, the present invention relates to a method for identifying CAP-Gly domain analogs that mimic the three-dimensional structure of the CAP-Gly domain. This method includes the steps of: producing a multiplicity of analog structures of the CAP-Gly domain by methods according to the seventh aspect of the invention; and selecting an analog structure represented by a three-dimensional representation wherein the three-dimensional configuration and spatial arrangement of regions involved in function of the CAP-Gly domain remain substantially preserved.

In a ninth aspect, the present invention relates to a method for producing an analog of a CAP-Gly domain that mimics the three-dimensional structure of the CAP-Gly domain. This method includes the steps of: determining the three-dimensional coordinates of atoms of an CAP-Gly domain; providing a computer having a memory means, a data input means, a visual display means, the memory means containing three-dimensional molecular simulation software operable to retrieve coordinate data from the memory means and to display a three-dimensional representation of a domain on the visual display means and being operable to produce a modified three-dimensional analog representation responsive to operator-selected changes to the chemical structure of the domain and to display the three-dimensional representation of the modified analog; inputting three-dimensional coordinate data of atoms of the CAP-Gly domain into the computer and storing the data in the memory means; inputting into the data input means of the computer at least one operator-selected change in chemical structure of the CAP-Gly domain; executing the molecular simulation software to produce a modified three-dimensional molecular representation of the analog structure; displaying the three-dimensional representation of the analog on the visual display means, whereby changes in three-dimensional structure of the CAP-Gly domain consequent on changes in chemical structure can be visually monitored; inputting operator-selected changes in the chemical structure of the CAP-Gly domain, executing the software to produce a modified three-dimensional molecular representation of the analog structure, and displaying the three-dimensional representation of the analog on the visual display means; selecting an analog structure represented by a three-dimensional representation wherein the three-dimensional configuration and spatial arrangement of regions involved in function of the CAP-Gly domain remain substantially preserved; synthesizing the selected analog by means of recombinant DNA technology; and determining the CAP-Gly domain function of the synthesized CAP-Gly domain analog, whereby an analog having the activity is a mimic of the three-dimensional structure of the CAP-Gly domain.

In a tenth aspect, the present invention relates to a method for identifying a potential ligand of a CAP-Gly domain containing protein. This method includes: using a three-dimensional structure of the CAP-Gly domain or portions thereof as defined by atomic coordinates of F53F4.3 according to Table 2; employing the three-dimensional structure to design or select the potential ligand; synthesizing the potential ligand; contacting the potential ligand with the CAP-Gly domain containing protein; and determining whether the potential ligand binds to the CAP-Gly domain containing protein.

In an eleventh aspect, the present invention relates to an analog of the CAP-Gly domain made by methods according to the sixth or ninth aspect of the invention.

In a twelfth aspect, the present invention relates to an analog structure of a CAP-Gly domain produced according to the seventh aspect of the invention.

In a thirteenth aspect, the present invention relates to a ligand of CAP-Gly domain containing polypeptide made according to the tenth aspect of the invention.

In a fourteenth aspect, the present invention relates to a method for identifying an interacting partner for a protein containing a CAP-Gly domain. This method includes the steps of: providing a CAP-Gly domain or analog thereof; contacting the CAP-Gly domain or analog thereof with potential interacting partners; and determining the presence of interaction between the CAP-Gly domain or analog thereof and the potential interacting partners, thereby identifying an interacting partner of the protein containing a CAP-Gly domain.

In a fifteenth aspect, the present invention relates to an apparatus for determining whether a compound will interact with a protein containing a CAP-Gly domain. This apparatus includes a memory that stores the three-dimensional coordinates and identities of the atoms of the CAP-Gly domain that together form a solvent-accessible surface and executable instructions; and a processor that executes instructions to receive three-dimensional structural information for a candidate compound, determine if the three-dimensional structure of the candidate compound is complementary to the structure of the solvent-accessible surface of the CAP-Gly domain, and output the results of the determination.

In a sixteenth aspect, the present invention relates to a computer-readable storage medium. This medium includes digitally-encoded structural data. The data includes the identity and three-dimensional coordinates of at least 6 amino acids of the CAP-Gly domain.

In a seventeenth aspect, the present invention relates to a repository of reference three-dimensional coordinates and software. The software is configured to; receive a subject set of coordinates which comprise a subject structure; compare each subject set of coordinates to the reference set of coordinates; calculate the root mean squared deviation of the subject set of coordinates from the reference set of coordinates; and compare the root mean squared deviation so obtained to limit values. If the root mean squared deviation calculated is less than or equal to the limit values, the subject structure is assigned a function based on the subject structure's similarity to CAP-Gly domain structure.

In an eighteenth aspect, the present invention relates to a method of determining relationships between two or more polypeptide structures. This method includes the steps of: obtaining a reference structure, wherein the reference structure is a structure of a polypeptide comprising the CAP-Gly domain or a portion thereof; obtaining at least one subject structure; determining a topology diagram for each of the reference and subject structures; comparing the topology diagram of the reference structure and the topology diagram of the subject structure; and assigning a relationship between the reference structure and any subject structure, wherein if the topology diagrams of the subject structures correspond to the topology diagram of the reference structure, the proteins have substantially the same protein fold.

In a nineteenth aspect, the present invention relates to polypeptides that include structure which is substantially the same as that of a polypeptide comprising the CAP-Gly domain or a portion thereof as indicated by the eighteenth aspect of the invention.

In a twentieth aspect, the present invention relates to method of identifying a compound that alters a function of a CAP-Gly domain containing protein. The method includes; providing a model of the structure of the CAP-Gly domain, studying the interaction of at least one candidate ligand with the model; selecting a compound which is predicted to act as a ligand; and determining that the selected compound will alter a function of a CAP-Gly domain containing protein.

In a twenty-first aspect, the present invention relates to a method of screening compounds to identify ligands with biological effects. The method includes: contacting a polypeptide comprising a CAP-Gly domain with at least one compound; assaying for a selected biological effect; assaying for the selected biological effect in the absence of the at least one compound; and comparing the level of the selected biological effect in the presence of the at least one compound to that in the absence of the at least one compound, whereby compounds are identified as ligands with biological effects when the level of the selected biological effect in the presence of the compound differs from the level of the selected biological effect in the absence of the compound.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1.(a) A section (Z=⅙) of the anomalous difference Patterson map at 3.5 Å resolution, contoured at 1.0σ, calculated from experimental X-ray diffraction data. The peaks observed in the map agree with the sulfur positions used in the phasing calculation. (b) A portion of the electron density map calculated with phases derived from sulfur anomalous diffraction data, followed by solvent-flattening phase extension and refinement. The map is contoured at 1.0σ around a section of the central sheet. The ribbon and bonds from the refined structure are shown for reference, with key residues labeled.

FIG. 2. Structure of the CAP-Gly domain of C. elegans F53F4.3 protein. Ribbon and surface plots are shown (Carson, “Ribbons” Methods in Enzymology 277: 493-505 (1997)). The surface plot adjacent to the ribbon is rotated approximately 90 degrees about the Y axis, and the next surface rotated 180 degrees more. (a) Temperature factors: The color-coding for the refined atomic B-factors is: yellow>36; orange>42; red>48. (b) Information content: The calculation is based on the 58 protein sequence alignment as described in the text (also see FIG. 3). The color-coding for the information content in bits is: green>2.75; blue>3.25; purple>3.75.

FIG. 3. Sequence alignment to the CAP-Gly domain of C. elegans F53F4.3. The amino acid sequences of 20 representative members of the 58 proteins aligned as described in the text. The 20 chosen had identities >30% and the most descriptive annotation, which is displayed at the end of each sequence. The observed secondary structure of the F53F4.3 is displayed as a cartoon above the sequences. The green, blue and purple rectangles along the secondary structure denote the information content of the entire 58 protein alignment, as in FIG. 2(b). The residue numbering above the sequences is for C. elegans F53F4.3. Note charged residues (D, E, H, K, R).

FIG. 4. The CAP-Gly structure. a, a schematic topology drawing of the CAP-Gly domain of C. elegans F53F4.3 protein. The β-strands are represented by arrows. There are three β-sheets formed by three, three, and two strands, respectively. The strand of β 2a and β 2b is continuous. b, a ribbon drawing of the three-dimensional structure of the CAP-Gly domain. The shading is the same as the topology drawing.

### BRIEF DESCRIPTION OF THE TABLES

Table 1 summarizes the X-ray crystallography data sets and refinement parameters of the structure of the F53F4.3 polypeptide which contains the CAP-Gly domain.

Table 2 provides the atomic structure coordinates of the F53F4.3 polypeptide, which contains the CAP-Gly domain, as determined by X-ray crystallography. The amino acids represented in Table 2 correspond to the amino acid sequence of SEQ ID NO: 1.

### DESCRIPTION OF THE PREFERRED EMBODIMENTS

Before the present compounds, compositions, articles, devices, and/or methods are disclosed and described, it is to be understood that this invention is not limited to specific methods, specific solutions, or to particular devices, as such may, of course, vary. It is also to be understood that the terminology used herein is for the purpose of describing particular embodiments only and is not intended to be limiting. Throughout the specification and claims, reference will be made to a number of terms which shall be defined to have the following meanings:

As used in the specification and the appended claims, the singular forms “a,” “an” and “the” include plural referents unless the context clearly dictates otherwise. Thus, for example, reference to “solvent” includes mixtures of solvents, and the like.

Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

“Amino acid,” as used herein, means the typically encountered 20 amino acids which make up polypeptides. In addition, it further includes less typical constituents which are both naturally occurring, such as, but not limited to formylmethionine and selenocysteine, analogs of typically found amino acids and mimetics of amino acids or amino acid functionalities.

“Analogous,” as used herein, particularly to describe a structure, means that the structure has characteristic properties like that of another structure, even though there may be substantial differences between the structures as a whole or between significant portions of the structures.

“Analog,” as used herein, has the commonly accepted meaning within the art. In particular, it refers to those compounds which have certain similarities in structure, function and/or properties to the species of which they are an analog. By way of illustration, the commonly known nucleoside analogs such as AZT, ddI, ddC, and d4T have both structural and functional similarity to normal nucleosides. Similar relationships between polypeptides or small molecule compounds and their corresponding analogs are also recognized by those of skill in the art.

“Mimetic,” as used herein, includes a chemical compound, or an organic molecule, or any other mimetic, the structure of which is based on or derived from a binding region of a polypeptide or ligand. For example, one can model predicted chemical structures to mimic the structure of a binding region, such as a binding loop of a peptide. Such modeling can be performed using standard methods (see for example, Zhao et al., Nat. Struct. Biol. 2: 1131-1137 (1995)). Mimetics identified by method such as this can be further characterized as having the same binding functions as the originally identified molecule of interest according the binding assays or modeling methods described herein. Mimetics can also be selected from combinatorial chemical libraries in much the same way that peptides are (Ostresh et al., Proc. Natl. Acad. Sci. U.S.A. 91: 11138-11142 (1994); Dorner et al., Bioorg. Med. Chem. 4: 709-715 (1996); Eichler et al., Med. Res. Rev. 15: 481-496 (1995); Blondelle et al., Biochiem. J. 313: 141-147 (1996); Perez-Paya et al., J. Biol. Chem. 271: 4120-4126 (1996)). Mimetics can also be designed on the basis of previous identified structures as is appreciated by those of skill in the art.

“Bind,” as used herein, means the well-understood interaction between two species. For example, the interaction between a polypeptide and a ligand or the interaction between a protein and a dye molecule. “Specifically bind,” as used herein, describes interactions between species wherein a member of the binding pair does not substantially cross react with other species not identical, or substantially similar to, or analogous to the other member of the binding pair. Specific binding is often associated with a particular set of interactions which form between the members of the binding pair.

“Domain,” as used herein, has the well-known meaning of the art used to classify and characterize protein structure. As the term is normally used, domains are considered to be compact, local, semi-independent units of protein structure. In a multi-domain protein, the domains can make up functionally and structurally distinct modules. These modules are usually formed from a single continuous segment of a polypeptide chain, a region of amino acid sequence.

“Deletion,” as used herein, refers to a change in either amino acid or nucleotide sequence in which one or more amino acid or nucleotide residues, respectively, are absent.

“Insertion” or “addition,” as used herein, refers to a change in an amino acid or nucleotide sequence resulting in the addition of one or more amino acid or nucleotide residues, respectively, as compared to the naturally occurring molecule.

“Substitution,” as used herein, refers to the replacement of one or more amino acids or nucleotides by different amino acids or nucleotides, respectively.

“Isolated,” as used herein refers to material, such as a nucleic acid or a protein, which is: (1) substantially or essentially free from components which normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a locus in the cell (e.g., genome or subcellular organelle) not native to a material found in that environment. The alteration to yield the synthetic material can be performed on the material within or removed from its natural state.

“Purified,” as used herein, refers to species, such as polypeptides, that are removed from their natural environment, isolated or separated, and are at least 60% free from other components with which they are normally associated or components similar to those with which they are normally associated. It is preferable that they be more free from other components than to be less free from other components. For example, more preferably they are more than 70%, 75%, 80%, 85%, 90%, 95%, 97%, 98%, or 99% free from other components.

“Substantially similar,” as used herein, refers in one aspect to polypeptides or portions thereof which have structures that are closely related to a referent polypeptide. When it is used in this context, substantially similar includes accommodation or specific differences mandated by specific differences between the species compared. For example, two polypeptide structures having the same structural motif, but with different amino acid sequence, are substantially similar. Likewise, two polypeptide structures having the same overall motif, but wherein there are regions of variance between the two structures can also be classified as substantially similar depending upon the degree of variance and the fraction of the structure over which it occurs. Structures with similarity between them such that they have RMSD of 3 Å or less (e.g., having RMSD of 2.5, 2, 1.5, 1 Å or less) are substantially similar. As will be recognized by those of skill in the art, substantially similar may also be used to refer to properties of different species.

“Topology,” as used herein, denotes specific characteristics of a protein structure. These characteristics include, but are not limited to, how the strands and helices are related sequentially in the folded chain, and the nature of the loops which connect them.

“B,” as used herein, means the thermal factor, i.e., temperature factor, that, measures movement of the atom around its atomic center. The B-factor is given by: Bᵢ=8π²Uᵢ² where Uᵢ² is the mean square displacement of atom I (as described in “Protein Crystallography” T. L. Blundell & L. N. Johnson, Academic Press, Inc., London (1976)).

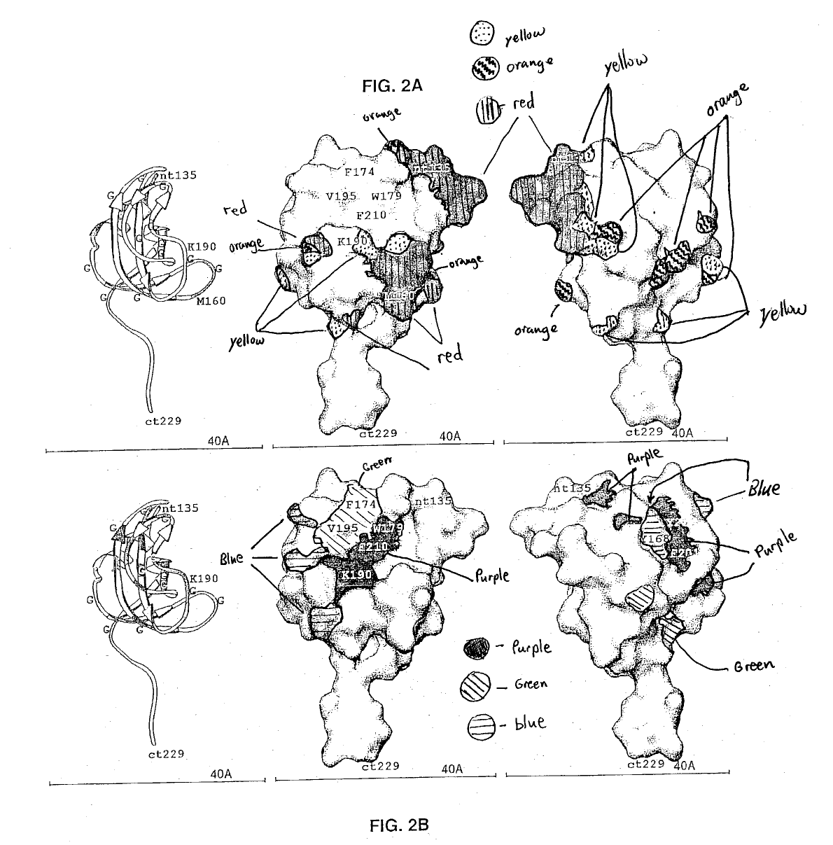

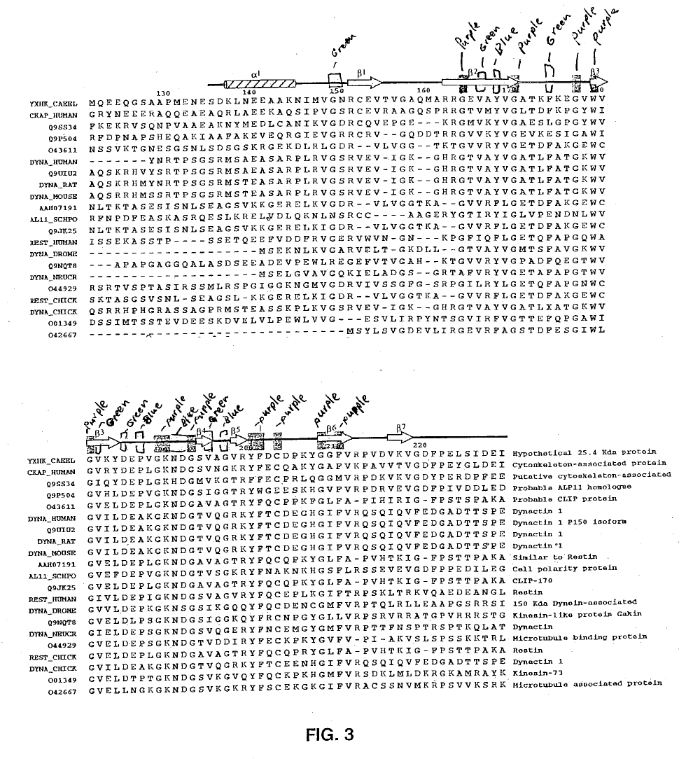

Amino acid substitutions, deletions and additions which do not significantly interfere with the three-dimensional structure of the CAP-Gly domain will depend, in part, on the region of the CAP-Gly domain where the substitution, deletion or addition occurs. In more variable portions of the structure, such as those shown in FIG. 3, non-conservative, as well as conservative substitutions, may be tolerated without significantly disrupting the three-dimensional structure of the CAP-Gly domain. In more conserved regions, or regions containing significant secondary and tertiary structure, such as those shown in FIGS. 2 and 3, conservative amino acid substitutions are preferred.

Conservative amino acid substitutions are well-known in the art, and include substitutions made on the basis of similarity in polarity, charge, solubility, hydrophobicity, hydrophilicity, amphipathicity and other factors. It is further recognized by those of skill in the art that substitutions, additions or deletions of a small percentage of amino acids (typically less than 5%, more typically less than 1%) in an encoded sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (1), Leucine (L), Methionine (M), Valine (V); and 6) Phenylalanine (F), Tyrosine (Y), Tryptophan (W).

It will further be recognized that certain substitutions, additions and/or deletions can result from adoption of nucleic acid sequences that are advantageous for providing convenient cloning, restriction endonuclease and/or other features used by those of skill in the art. Further, there exist substitutions, addition and/or deletions which can be used to provide features useful in the purification of the polypeptide, such as is described herein with respect to a histidine tag but which applies equally well to other features.

Substitutions, additions and/or deletions to the CAP-Gly domain, and/or the sequence disclosed herein, which result in structures that are analogous to the structure described herein or portions thereof are specifically contemplated. Further, substitutions, additions and/or deletions to the CAP-Gly domain which result in structures which are substantially the same as the structure disclosed herein or portions thereof are also specifically contemplated.

It should be noted that structures comprising portions of the CAP-Gly domain, the disclosed structure of F53F4.3 protein, or portions thereof, need not retain the activity of the proteins or domains from which they are derived. It is specifically contemplated that certain mutants and variants of the disclosed structures will retain structures substantially similar to and or analogous to the disclosed structures, but will not have other features or activities that are typically associated with the non mutant or the non-varied species.

“Portion,” as used herein, refers to the common meaning of the term as used by those of skill in the art. Specifically, a portion of an amino acid sequence includes any other amino acid sequence which comprises the portion and any further sequence.

Structures of the polypeptides of the invention can be obtained by those methods disclosed in the Examples Section or by other means known to those of skill in the art. Some of the methods recognized by those of skill in the art require the use of crystals comprising the CAP-Gly domain. Crystals suitable for structure determination can be obtained by use of standard practices known to those of skill in the art. These include, but are not limited to, batch, liquid bridge, dialysis, vapor diffusion and hanging drop methods (see U.S. Pat. No. 5,942,428, incorporated herein by reference).

The crystals of the invention, and the atomic structure coordinates obtained therefrom such as those in Table 2, are useful. For example, the structure coordinates can be used as phasing models for determining the structures of additional members of the CAP-Gly domain containing proteins, of complexes between the CAP-Gly domain or other such domains and ligands which bind to the CAP-Gly domain.

The atomic level structure, as well as CAP-Gly domain containing polypeptides, of the invention can be used for ligand screening, modeling and design. For example, compounds that are not known ligands of proteins comprising the CAP-Gly domain can be brought into contact with CAP-Gly domain proteins, and the structure of complexes, if formed, can be elucidated. Use of the presently disclosed structure of the uncomplexed CAP-Gly structure can then be used to determine the structure of complexes formed between the CAP-Gly domain and compounds which interact with the CAP-Gly domain. Examples of such methods of ligand screening, modeling and design as applied to other proteins can be found in U.S. Pat. Nos. 6,297,021; 6,251,620; 5,856,116; 5,864,488; 5,834,228; 6,316,416; and 6,225,076, each incorporated herein by reference). The application of the methods described in each of these references, and others cited elsewhere herein, using the structure disclosed herein and adaptation to the specifics of CAP-Gly domain containing proteins as appropriate is also contemplated.

Structural coordinates, such as atomic coordinates, of this invention can be stored in a machine-readable form on machine-readable storage medium. Examples of such media include, but are not limited to, computer hard drive, diskette, DAT tape, CD-ROM, and the like. The information stored on this media can be used for display as a three-dimensional shape or representation thereof or for other uses based on the structural coordinates, the spatial relationships between atoms described by the structural coordinates or the three-dimensional structures that they define. Such uses can include the use of a computer capable of reading the data from the storage media and executing instructions to generate and/or manipulate structures defined by the data. Commonly used sets of instructions, i.e., computer programs, for viewing or otherwise manipulating structures include, but are not limited to; Midas (UCSF), MidasPlus (UCSF), MOIL (University of Illinois), Yummie (Yale University), Sybyl (Tripos, Inc.), Insight/Discover (Biosym Technologies), MacroModel (Columbia University), Quanta (Molecular Simulations, Inc.), Cerius (Molucular Simulations, Inc.), Alchemy (Tripos, Inc.), LabVision (Tripos, Inc.), Rasmol (Glaxo Research and Development), Ribbon (University of Alabama), NAOMI (Oxford University), Explorer Eyechem (Silicon Graphics, Inc.), Univision (Cray Research), Molscript-(Uppsala University), Chem-3D (Cambridge Scientific), Chain (Baylor College of Medicine), 0 (Uppsala University), GRASP (Columbia University), X-Plor (Molecular Simulations, Inc.; Yale University), Spartan (Wavefunction, Inc.), Catalyst (Molecular Simulations, Inc.), Molcadd (Tripos, Inc.), VMD (University of Illinois/Beckman Institute), Sculpt (Interactive Simulations, Inc.), Procheck (Brookhaven National Laboratory), DGEOM (QCPE), RE_VIEW (Brunel University), Modeller (Birbeck College, University of London), Xmol (Minnesota Supercomputing Center), Protein Expert (Cambridge Scientific), HyperChem (Hypercube), MD Display (University of Washington), PKB (National Center for Biotechnology Information, NIH), ChemX (Chemical Design, Ltd.), Cameleon (Oxford Molecular, Inc.), and Iditis (Oxford Molecular, Inc.).

Ligands as defined herein can include antibodies generated against peptides of the present invention or reactive against the polypeptides of the present invention. Use of these antibodies for the purposes of characterizing the polypeptides of the invention is contemplated. Use of these antibodies, that can bind to CAP-Gly domain containing proteins or polypeptides, to form antibody containing complexes is also contemplated. New chemical or recombinant ligands can be generated by identifying compounds that bind to CAP-Gly domain containing proteins, particularly those that bind to the CAP-Gly domain. Once a lead compound is identified that binds to the CAP-Gly domain containing protein, or more particularly the CAP-Gly domain itself, variants can be created and evaluated for use as a therapeutic agent. A wide variety of compounds can be screened for binding activity. Such molecules are generally identified by screening known or newly-generated libraries of compounds, by computer-assisted drug design (utilizing the structural coordinates provided by the present invention) or by a combination of these methods. A combinatorial chemical library is a collection of diverse chemical compounds generated by either chemical synthesis or biological synthesis, by combining a number of chemical “building blocks” such as reagents. For example, a linear combinatorial chemical library such as a polypeptide library can be formed by combining a set of chemical building blocks called amino acids in every possible way for a given compound length (i.e., the number of amino acids in a polypeptide compound).

Preparation and screening of combinatorial chemical libraries is well known to those of skill in the art. Such combinatorial chemical libraries include, but are not limited to, peptide libraries (see e.g., U.S. Pat. No. 5,010,175). Chemical libraries can also be generated that peptoids (PCT Pub. No. WO 91/19735), encoded peptides (PCT Pub. No. WO 93/20242), random bio-oligomers (PCT Pub. No. WO 92/00091), benzodiazepines (U.S. Pat. No. 5,288,514), diversomers such as hydantoins, benzodiazepines and dipeptides (Hobbs et al., Proc. Nat. Acad. Sci. USA 90: 6909-6913 (1993)), vinylogous polypeptides (Hagihara et al., J. Amer. Chem. Soc. 114: 6568 (1992)), non-peptidal peptidomimetics with β-D-glucose scaffolding (Hirschmann et al., J. Amer. Chem. Soc. 114: 9217-9218 (1992), analogous organic syntheses of small compound libraries (Chen et al., J. Amer. Chem. Soc. 116: 2661 (1994)), oligocarbamates (Cho et al., Science, 261: 1303 (1993)), and/or peptidyl phosphonates (Campbell et al., J. Org. Chem. 59: 658 (1994)), nucleic acid libraries, peptide nucleic acid libraries (see e.g., U.S. Pat. No. 5,539,083), antibody libraries (U.S. Pat. No. 5,593,853), small organic molecules libraries (see e.g., benzodiazepines; isoprenoids, U.S. Pat. No. 5,569,588; thiazolidinones and metathiazoanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like).

Devices for the preparation of combinatorial libraries are commericially available (see e.g., 357 MPS, 390 MPS, Advanced Chem Tech, Louisville, Ky.; Symphony, Rainin, Woburn, Mass.; 433A, Applied Biosystems, Foster City, Calif.; 9050 Plus, Millipore, Beford, Mass.).

Solution phase chemistries can also be used to generate suitable libraries. Particularly, when accomplished using robotic systems. These systems include, but are not limited to, those manufactured and/or available from Takeda Chemical industries Ltd., Zymate II from Zymark Corp., and Orca from HP. The nature of these devices and their modification to accomplish necessary operations will be apparent to those of skill in the art. However, the present invention can also be accomplished using any number of the numerous combinatorial libraries that are themselves commercially available (see e.g., ComGenex, Princeton, N.J.; Asinex, Moscow, Ru; Tripos, Inc., St. Louis, Martek Biosciences, Columbia, Md., etc.).

Identification of ligands can include detection of direct binding of candidate compounds to CAP-Gly domain containing proteins. Identification of ligands, or the characterization of identified ligands as having an activity, can be accomplished using cell-based assays. Cell-based assays can be in vivo, wherein the cells used are living cells, including cells in an animal and cells ex vivo. Ex vivo refers to assays that are performed using an intact membrane that is outside the body, e.g., explants, cultured cell lines, transformed cell lines, primary cell lines, and extracted tissue, e.g., blood. In vitro assays, those that do not require the presence of cells with an intact membrane, can include screening for binding using isolated and/or recombinant CAP-Gly domain containing proteins, but also includes any assay wherein cellular contents have been removed from the constraints of an intact membrane.

Assays to determine activity of compounds can include assays for formation of aggresomes. Assays can be in vivo, ex vivo, or in vitro. Compounds to be assayed can include those identified as ligands of CAP-Gly domain containing proteins. If identified as ligands, they can be ligands that bind to the CAP-Gly domain. For example, a collection of compounds characterized as potential ligands or identified as ligands can be contacted with cells that have been infected with a virus, such as ASFV, that forms viral factories. Staining or detection of viral proteins, thereby allowing monitoring of viral assembly, can be accomplished using practices as are known in the art. Alternatively, monitoring of complete viral particles formed can be used to determine an effect on viral assembly. One such method for monitoring aggresome formation, as is known in the art, is described in Heath et al., “Aggresomes resemble sites specialized for virus assembly,” J. Cell Biol. 153: 449-455 (2001), incorporated herein by reference for its teachings regarding assays.

The antibodies of the present invention which specifically bind the polypeptides of the present invention or portions thereof can include polyclonal and monoclonal antibodies which can be intact immunoglobulin molecules, chimeric immunoglobulin molecules, or Fab or F(ab′)₂ fragments. Such antibodies and antibody fragments can be produced by techniques well known in the art which include those described in Harlow and Lane (“Antibodies: A Laboratory Manual” Cold Spring Harbor Laboratory, Cold Spring Harbor, N.Y., (1989)) and Kohler et al. (Nature 256: 495-97 (1975); and U.S. Pat. Nos. 5,545,806, 5,569,825 and 5,625,126, incorporated herein by reference. The antibodies can be of any isotype IgG, IgA, IgD, IgE and IgM.

The present invention can also include single chain antibodies (ScFv), comprising linked VH and VL domains and which retain the conformation and specific binding activity of the native idiotype of the antibody. Such single chain antibodies are well known in the art and can be produced by standard methods. (see, e.g., Alvarez et al., Hum. Gene Ther. 8: 229-242 (1997)).

The antibodies can be produced against peptides of the known amino acid sequence of CAP-Gly domains, or peptides comprising a CAP-Gly domain, which can be easily identified to be immunogenic peptides according to methods well known in the art for identifying immunogenic regions in an amino acid sequence. It is preferred that the antibodies be specific for CAP-Gly domain specific sequence and/or structures.

Conditions whereby an antigen/antibody complex can form as well as assays for the detection of the formation of an antigen/antibody complex and quantitation of the detected protein are standard in the art. Such assays can include, but are not limited to, Western blotting, immunoprecipitation, immunofluorescence, immunocytochemistry, immunohistochemistry, fluorescence activated cell sorting (FACS), fluorescence in situ hybridization (FISH), immunomagnetic assays, ELISA, ELISPOT (Coligan et al., eds., Current Protocols in Immunology, Wiley, N.Y. (1995)), agglutination assays, flocculation assays, cell panning, etc., as are well known to those of skill in the art.

The antibody of this invention can be bound to a substrate (e.g., beads, tubes, slides, plates, nitrocellulose sheets, etc.) or conjugated with a detectable moiety or both bound and conjugated. The detectable moieties contemplated for the present invention can include, but are not limited to, an immunofluorescence moiety (e.g., fluorescein, rhodamine), a radioactive moiety (e.g., ³²P, ¹²⁵I, ³⁵S), an enzyme moiety (e.g., horseradish peroxidase, alkaline phosphatase), a colloidal gold moiety and a biotin moiety. Such conjugation techniques are standard in the art (see, e.g., Harlow and Lane, “Antibodies, A Laboratory Manual” Cold Spring Harbor Publications, New York, (1988); Yang et al., Nature 382: 319-324 (1996)). As recognized by those of skill in the art, a detectable moiety or label can be a composition detectable by spectroscopic, photochemical, biochemical, immunochemical or chemical methods. The antibodies for the CAP-Gly domain or fragments, analogs or mimetics thereof can be used to bind to CAP-Gly domain containing polypeptides in vitro or in vivo. When the antibody is coupled to a label which is detectable but which does not interfere with binding to the CAP-Gly domain or fragments thereof, the antibody can be used to identify the presence or absence of accessible CAP-Gly domains. Labels can be coupled either directly or indirectly to the disclosed antibodies. One example of indirect coupling is by use of a spacer moiety. These spacer moieties, in turn, can be either insoluble or soluble (Diener et al., Science 231: 148 (1986)).

There are many different labels and methods of labeling known to those of ordinary skill in the art. Examples of the types of labels which can be used in the present invention include enzymes, radioisotopes, fluorescent compounds, chemiluminescent compounds, and bioluminescent compounds. Those of ordinary skill in the art will know of other suitable labels for binding to the monoclonal antibody, or will be able to ascertain such, using routine experimentation. Furthermore, the binding of these labels to the monoclonal antibody of the invention can be done using standard techniques common to those of ordinary skill in the art.

For in vivo detection, radioisotopes may be bound to immunoglobin either directly or indirectly by using an intermediate functional group. Intermediate functional groups which often are used to bind radioisotopes which exist as metallic ions to immunoglobins are the bifunctional chelating agents such as diethylenetriaminepentacetic acid (DTPA) and ethylenediaminetetraacetic acid (EDTA) and similar molecules.

For any aspect of the invention where delivery of a compound, protein or other element to an organism, tissue, or cell is required, the use of appropriate methods as known to those of skill in the art is contemplated. Vectors (e.g., retroviruses, adenoviruses, lipsomes, etc.) containing nucleic acids can be administered directly to the organism, tissue or cell for transduction of cells. Alternatively, naked DNA can be administered.

Administration can be by any of the routes normally used for introducing a molecule into ultimate contact with blood or tissue cells, as described below. If a nucleic acid is to be used, for example, to induce the production of a desired polypeptide, they can be administered in any suitable manner, preferably with pharmaceutically acceptable carriers. Suitable methods of administering such nucleic acids are available and well known to those of skill in the art and, although more than one route can be used to administer a particular composition, a particular route can often provide advantages. Optimization of methods of administration and of the compounds administered are within the skill of those in the art and are hereby contemplated.

Administration of compounds or ligands can be by any of the routes normally used to introduce a compound into ultimate contact with the tissue to be affected. The compounds can be administered in any suitable manner, preferably with pharmaceutically acceptable carriers. Suitable methods of administering such compounds are available and well known to those of skill in the art. Pharmaceutically acceptable carriers are determined in part by the particular composition being administered, as well as by the particular method used to administer the composition. Accordingly, there can be a wide variety of suitable formulations (e.g., Remington's Pharmaceutical Sciences).

In one aspect, the present invention relates to a polypeptide that includes, amino acid sequence of the CAP-Gly domain or a portion thereof and heterologous amino acid sequence. The heterologous sequence can be from a different protein, a different species or can be sequence not derived from any other known amino acid sequence.

The polypeptide can include amino acid sequence of at least 5, 6, 7, 8, 9, 10, 12, 15, 20, 25, 30, 40, 50, 75, 100 or 150 contiguous amino acid residues derived from the CAP-Gly domain. The contiguous amino acid residues can be those described by SEQ ID NO: 1 or a portion thereof. The polypeptide can be an isolated polypeptide or a purified polypeptide. The polypeptide can have a CAP-Gly domain with structure analogous to the structure of the CAP-Gly domain of F53F4.3 protein or can structure substantially the same as the structure of the CAP-Gly domain of F53F4.3 protein.

Methods of designing, generating and expressing chimeric proteins containing heterologous sequence are known to those of skill in the art (see Molecular Cloning: A Laboratory Manual, 2nd Ed., Sambrook, Fritsch and Maniatis, Cold Spring Harbor Laboratory, Cold Spring Harbor, N.Y., 1989; and Current Protocols in Molecular Biology, Ausubel et al., John Wiley and Sons, New York 1987 (updated quarterly)). For example, production of the proteins can include cultivating cells of microorganisms that have been transformed by nucleic acids, such as, but not limited to, DNA comprising sequences that encode the amino acid sequence of the protein. If this is done under conditions allowing the expression of the protein encoded by the nucleic acid, subsequent purification of the protein using methods known to those of skill in the art yield the protein.

In a second aspect, the present invention relates to an isolated polypeptide containing an amino acid sequence according to amino acid residues 135 to 229 of a cytoskeletal-associated protein with structure analogous to the structure of the CAP-Gly domain of F53F4.3 protein. The sequence according to amino acid residues 135 to 229 is designated as SEQ ID NO: 1. The sequence of SEQ ID NO: 1 is as follows:

SER ASP LYS LEU ASN GLU GLU ALA ALA LYS ASN ILE
MET VAL GLY ASN ARG CYS GLU VAL THR VAL GLY ALA
GLN MET ALA ARG ARG GLY GLU VAL ALA TYR VAL GLY
ALA THR LYS PHE LYS GLU GLY VAL TRP VAL GLY VAL
LYS TYR ASP GLU PRO VAL GLY LYS ASN ASP GLY SER
VAL ALA GLY VAL ARG TYR PHE ASP CYS ASP PRO LYS
TYR GLY GLY PHE VAL ARG PRO VAL ASP VAL LYS VAL
GLY ASP PHE PRO GLU LEU SER ILE ASP GLU ILE

Alternatively, the structure of the polypeptide can be substantially the same as the structure of the CAP-Gly domain of F53F4.3 protein. The polypeptide can include the amino acid sequence GKNDG (SEQ ID NO: 2), GKHDG (SEQ ID NO: 3), GKNSG (SEQ ID NO: 4) or GKHSG (SEQ ID NO: 5). If these sequences are present, they can be located in the portion of the polypeptide having structure analogous to or structure substantially the same as the CAP-Gly domain.

In a third aspect, the present invention relates to a crystal of the CAP-Gly domain, wherein the space group of the crystal is P6122 and unit cell dimensions of the crystal are: about a=64±3 Å, b=64±3 Å, and c=102±3 Å; about a=64±2 Å, B=5±2 Å, and c=102±2 Å; or about a=64±1 Å, b=64±1 Å, and c=102±1 Å. The CAP-Gly domain can have a three-dimensional structure characterized by the atomic structure coordinates of Table 2. The crystals can be formed from polypeptides having heterologous sequence.

In a fourth aspect, the present invention relates to a method of characterizing protein structures. This method can include the step of determining the three-dimensional structure of the CAP-Gly domain. It can include the step of determining the three-dimensional structure of an experimental protein. It can include the step of comparing the three-dimensional structure of the experimental protein to the three-dimensional structure of the CAP-Gly domain. It can include recording variances between the three-dimensional structure of the CAP-Gly domain and the experimental protein. The three-dimensional structure of the CAP-Gly domain can be derived from the structure of polypeptides containing heterologous sequence. The three-dimensional structure can be derived from a crystal of the CAP-Gly domain, wherein the space group of the crystal is P6122 and unit cell dimensions of the crystal are about a=64 Å, b=64 Å, and c=102 Å. The three-dimensional structure can be the structure defined by the atomic structure coordinates of Table 2.

In a fifth aspect, the present invention relates to a method of evaluating two or more experimental proteins in respect to the CAP-Gly domain. This method can include the step of evaluating the variances between the three-dimensional structure of each experimental protein and the three-dimensional structure of the CAP-Gly domain. It can include ranking the experimental protein with the least variance from the structure of CAP-Gly domain as being most similar to the CAP-Gly domain. It can include ranking additional experimental proteins in respect to their variance from the structure of the CAP-Gly domain.

In a sixth aspect, the present invention relates to a method for generating analogs of polypeptides that contain the CAP-Gly domain. This method can include determining the structure of a CAP-Gly domain. It can include selecting a polypeptide containing an amino acid sequence that maintains a CAP-Gly domain structure. It can include generating an analog polypeptide containing the amino acid sequence that maintains the CAP-Gly domain structure.

In a seventh aspect, the present invention relates to a method for determining whether an analog of the CAP-Gly domain will have an altered three-dimensional structure as compared to the CAP-Gly domain. This method can include determining the three-dimensional coordinates of atoms of a CAP-Gly domain. It can include providing a computer having a memory means, a data input means and a visual display means. The memory means can contain three-dimensional molecular simulation software operable to retrieve coordinate data from the memory means and operable to display a three-dimensional representation of a molecule on the visual display means. Further, the memory means can be operable to produce a three-dimensional representation of an analog of the molecule responsive to operator-selected changes to the chemical structure of the molecule and to display the three-dimensional representation of the analog. It can include inputting three-dimensional coordinate data of the atoms of the CAP-Gly domain into the computer and storing the data in the memory means. It can include displaying a three-dimensional representation of the CAP-Gly domain on the visual display means. It can include inputting into the data input means of the computer at least one operator-selected change in chemical structure of the CAP-Gly domain. It can include executing the molecular simulation software to produce a modified three-dimensional molecular representation of the analog structure. It can include displaying the three-dimensional representation of the analog on the visual display means, whereby changes in three-dimensional structure of the Cap-Gly domain consequent on changes in chemical structure can be visually determined. Selection of the analog structure can include the displaying on the visual display means the three-dimensional structure of both the original CAP-Gly domain and the CAP-Gly domain analog. The selection can include visually comparing the configuration and spatial arrangement of the CAP-Gly domain and selecting an analog structure wherein the domains are substantially the same.

In an eighth aspect, the present invention relates to a method for identifying CAP-Gly domain analogs that mimic the three-dimensional structure of the CAP-Gly domain. This method can include the step of producing a multiplicity of analog structures of the CAP-Gly domain by methods according to the seventh aspect of the invention. This method can include selecting an analog structure represented by a three-dimensional representation wherein the three-dimensional configuration and spatial arrangement of regions involved in function of the CAP-Gly domain remain substantially preserved.

In a ninth aspect, the present invention relates to a method for producing an analog of a CAP-Gly domain that mimics the three-dimensional structure of the CAP-Gly domain. This method can include the step of determining the three-dimensional coordinates of atoms of a CAP-Gly domain. It can also include providing a computer having a memory means, a data input means and a visual display means. The memory means can contain three-dimensional molecular simulation software operable to retrieve coordinate data from the memory means and operable to display a three-dimensional representation of a domain on the visual display means. This software can be operable to produce a modified three-dimensional analog representation responsive to operator-selected changes to the chemical structure of the domain and be operable to display the three-dimensional representation of the modified analog. This method can also include inputting three-dimensional coordinate data of atoms of the CAP-Gly domain into the computer and storing the data in the memory means, inputting into the data input means of the computer at least one operator-selected change in chemical structure of the CAP-Gly domain, executing the molecular simulation software to produce a modified three-dimensional molecular representation of the analog structure, displaying the three-dimensional representation of the analog on the visual display means, whereby changes in three-dimensional structure of the CAP-Gly domain consequent on changes in chemical structure can be visually monitored, inputting operator-selected changes in the chemical structure of the CAP-Gly domain, executing the software to produce a modified three-dimensional molecular representation of the analog structure, and displaying the three-dimensional representation of the analog on the visual display means. This method can also include selecting an analog structure represented by a three-dimensional representation wherein the three-dimensional configuration and spatial arrangement of regions involved in function of the CAP-Gly domain remain substantially preserved, synthesizing the selected analog by means of recombinant DNA technology, and determining the CAP-Gly domain function of the synthesized CAP-Gly domain analog, whereby an analog having the activity is a mimic of the three-dimensional structure of the CAP-Gly domain. Examples of such function and activity by which to determine whether an analog is a mimic includes, but is not limited to, the ability to bind ligands normally bound by the CAP-Gly domain.

In a tenth aspect, the present invention relates to a method for identifying a potential ligand of a CAP-Gly domain containing protein. This method can include using a three-dimensional structure of the CAP-Gly domain or portions thereof as defined by atomic coordinates of F53F4.3 according to Table 2 to design or select a potential ligand. It can include employing the three-dimensional structure to design or select the potential ligand. It can include synthesizing the potential ligand. It can include contacting the potential ligand with the CAP-Gly domain containing protein and determining whether the potential ligand binds to the CAP-Gly domain containing protein. When the three-dimensional structure is used to design or select a ligand, the method can include identification of chemical functionalities capable of associating with the CAP-Gly domain based on chemical principles and the structure. It can also include assembling the identified chemical functionalities into a single molecule to provide the structure of the CAP-Gly domain potential ligand. When the three-dimensional structure is used to design potential ligands, the potential ligands can be designed de novo or a known compound can be modified to provide a potential ligand. The CAP-Gly domain, from which the structure and/or coordinates are derived, can consist essentially of sequence corresponding to amino acid residue 135 through amino acid residue 229 of F53F4.3 from Candida elegans. If atomic coordinates are used, the atomic coordinates can be those obtained when a crystal having the space group of P6122 and unit cell dimensions of about a=64 Å, b=64 Å, and c=102 A are used to determine the structure. The atomic coordinates can also be those shown in Table 2.

In an eleventh aspect, the present invention relates to an analog of the CAP-Gly domain made by methods according to the sixth or ninth aspect of the invention.

In a twelfth aspect, the present invention relates to an analog structure of a CAP-Gly domain produced according to the seventh aspect of the invention.

In a thirteenth aspect, the present invention relates to a ligand of CAP-Gly domain containing polypeptide made according to the tenth aspect of the invention.

In a fourteenth aspect, the present invention relates to a method for identifying an interacting partner for a protein containing a CAP-Gly domain. This method can include the steps of providing a CAP-Gly domain or analog thereof and contacting the CAP-Gly domain or analog thereof with potential interacting partners. It can also include determining the presence of interaction between the CAP-Gly domain or analog thereof and the potential interacting partners. If interaction is detected, this can identify an interacting partner of the protein containing a CAP-Gly domain. The CAP-Gly domain or analog thereof can be a part of a polypeptide containing heterologous sequence.

In a fifteenth aspect, the present invention relates to an apparatus for determining whether a compound will interact with a protein containing a CAP-Gly domain. This apparatus can includes a memory that stores the three-dimensional coordinates and identities of the atoms of the CAP-Gly domain that together form a solvent-accessible surface and executable instructions. The apparatus can also include a processor that executes instructions to receive three-dimensional structural information for a candidate compound, determine if the three-dimensional structure of the candidate compound is complementary to the structure of the solvent-accessible surface of the CAP-Gly domain, and output the results of the determination. The three-dimensional coordinates and identities of atoms of the CAP-Gly domain can be those derived from the structure of amino acid residue 135 through amino acid residue 229 of F53F4.3 from Candida elegans. Further, the set of three-dimensional coordinates and identities of atoms of the CAP-Gly domain can be those derived from a crystal having the space group of P6122 and unit cell dimensions of approximately a=b=64 Å and c=102 Å. Still further, the three-dimensional coordinates and identities of atoms of the CAP-Gly domain can be the atomic coordinates in Table 2.

In a sixteenth aspect, the present invention relates to a computer-readable storage medium. This medium includes digitally-encoded structural data. The data can include the identity and three-dimensional coordinates of atoms of at least 5 amino acids of the CAP-Gly domain. The data can include the identity and/or three-dimensional coordinates of atoms from 7, 8, 9, 10, 11, 12, 13, 14, 15, 16, 17, 18, 19, 20, 25, 30, 35, 40, 50, 60, 70, 80, 90, 100 or 150 amino acid residues from a polypeptide containing the CAP-Gly domain or from a polypeptide that consists of a portion of the CAP-Gly domain. The structural data can contain the identity and the three-dimensional coordinates of atoms from 7, 8, 9, 10, 11, 12, 13, 14, 15, 16, 17, 18, 19, 20, 25, 30, 35, 40, 50, 60, 70, 80, 90, 100 or 150 amino acid residues from a polypeptide containing the CAP-Gly domain or from a polypeptide that consists of a portion of the CAP-Gly domain. The data contained in computer-readable storage medium can include the atomic coordinates in Table 2 or a portion thereof.

In a seventeenth aspect, the present invention relates to a repository of reference three-dimensional coordinates and software. The software is configured to; receive a subject set of coordinates which comprise a subject structure; compare each subject set of coordinates to the reference set of coordinates; calculate the root mean squared deviation of the subject set of coordinates from the reference set of coordinates; and compare the root mean squared deviation so obtained to limit values. If the root mean squared deviation calculated is less than or equal to the limit values, the subject structure is assigned a function based on the subject structure's similarity to CAP-Gly domain structure. The reference coordinates can be the coordinates shown in Table 2 or a portion thereof. The limit values associated with the reference coordinates can correspond to values less than or equal to 3 Å, 2.5 Å, 2 Å, 1.5 Å, 1 Å, 0.5 Å, 0.2 Å, or 0.1 Å in root mean squares deviation.

In an eighteenth aspect, the present invention relates to a method of determining relationships between two or more polypeptide structures. This method includes the steps of obtaining a reference structure and at least one subject structure, wherein the reference structure is a structure of a polypeptide containing the CAP-Gly domain or a portion thereof. This method includes determining a topology diagram for each of the reference and subject structures. This method also includes comparing the topology diagram of the reference structure and the topology diagram of the subject structure and, on the basis of this comparison, assigning a relationship between the reference structure and any subject structure. Any such assignment can be based on whether or not the topology diagrams of the subject structures correspond to the topology diagram of the reference structure. The assignment of a relationship between proteins can include an indication that the proteins have substantially the same protein fold. It can also include an indication that the proteins have analogous protein folds.

The reference structure used for determining relationships between proteins and the presence, or absence, of the CAP-Gly domain motif can include the use of a structure defined by the atomic coordinates of Table 2 as a reference structure.

The determination of topology diagrams can include consideration of secondary structural elements, spatial adjacency of secondary structural elements within the observed protein fold and the approximate orientation of secondary structural elements. The determination of topology diagrams can neglect the length of loop elements or their structure. The determination of topology diagrams can also neglect the spatial orientations of secondary structural elements.

As will be recognized by those of skill in the art, the determination of topology diagrams such as those contemplated here can be accomplished by a number of different particular methods. Further, the method by which a particular topology is displayed or denoted can also be accomplished by a number of different particular methods. Thus, in a more basic form, a portion of the method described herein includes learning distinguishing patterns of proteins, those structural aspects which make them relatively unique, and comparing these to one another, wherein those which have the same distinguishing patterns have similar, or the same, structural aspects.

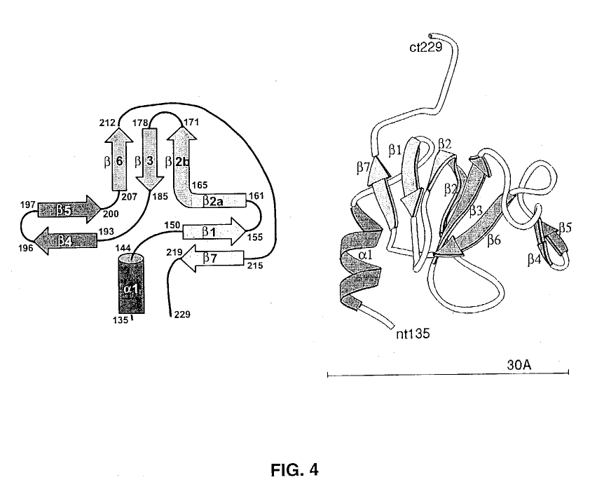

Methods of learning and comparing the structural aspects of proteins can include generating topology diagrams which convey combinations of a few secondary structure elements with specific geometric arrangements. Principles of protein structure related to the scientific basis for using these simplified diagrams, as well as methods of generating these simplified diagrams can be found throughout the literature (for e.g., Branden et al., “Introduction to Protein Structure” Garland Publishing, Inc., NY & London, 1991; Westhead et al., Prot. Sci. 8: 897-904 (1999); Gilbert et al., Bioinformatics 15: 317-326 (1999); and references cited therein). As is known to those of skill in the art, the determination of topology diagrams can be accomplished manually. However, it is often done using computer algorithms and software. One particular method of determining topology diagrams is the use of TOPS protein topology search, pattern discovery and structure comparison. Other aspects, including description of the principles and methods to apply those principles that allow those of skill in the art to practice the techniques of topology diagramming, are described in Richardson (“Beta-sheet topology and the relatedness of proteins” Nature 268: 495-500 (1977); “The anatomy and taxonomy of protein structure” Advances in Protein Chemistry 34: 167-339 (1981)). FIG. 4 illustrates some features common to most topology diagrams.

In a nineteenth aspect, the present invention relates to a polypeptide that includes any amino acid sequence that adopts structure substantially similar to that of a polypeptide comprising the CAP-Gly domain or a portion thereof. Polypeptides can be identified as being ones that adopt structure substantially similar to those comprising the CAP-Gly domain or a portion thereof by use of the concept of topology diagrams as described above. The amino acid sequence of the polypeptide that has some structure substantially similar to that of at least a portion of the CAP-Gly domain can be of different lengths. For example, the length of any amino acid sequence within the polypeptide that has a structure substantially similar to that observed in the CAP-Gly domain can be greater than 5, 6, 7, 8, 9, 10, 12, 14, 16, 18, 20, 25, 30, 35, 40, 50, 60, 70, 80 or 90 contiguous residues in length. Alternatively, the length of any amino acid sequence within the polypeptide that has a structure substantially similar to that observed in the CAP-Gly domain can be less than 100, 90, 80, 70, 60, 50, 40, 35, 30, 25, 20, 18, 16, 14, 12, 10, 9, 8, 7, or 6 contiguous residues in length. Further, it is contemplated that more than one amino acid sequence in a polypeptide can adopt structure substantially similar to that of a polypeptide comprising the CAP-Gly domain or a portion thereof. For example, by way of illustration, a polypeptide of 120 amino acid residues could contain three regions of 15, 20, and 7 residues in length that are each substantially similar to portions of the structure of the CAP-Gly domain.

The CAP-Gly domain structure against which other polypeptide structures are compared to determine if they are substantially similar to the CAP-Gly domain can be the structure defined by atomic coordinates from Table 2.

In a twentieth aspect, the present invention relates to method of identifying a compound that alters a function of a CAP-Gly domain containing protein. The method includes; providing a model of the structure of the CAP-Gly domain, studying the interaction of at least one candidate ligand with the model; selecting a compound which is predicted to act as a ligand; and determining that the selected compound will alter a function of a CAP-Gly domain containing protein. The CAP-Gly domain structure used in the method can be a structure according to that described in Table 2. Studying the interaction can include studying the interaction of a ligand with selected amino acids from the CAP-Gly domain. Selected amino acids can include amino acids selected from the group consisting of sequence according to Gly189 to Gly193, of sequence according to Val156 to Met160, Arg162, Tyr168, Phe174, Trp179, Lys190, Asn191, Val195, Tyr200, Phe201, Gly209, Phe210, and Val211 of F54F4.3, homologs, and conservative variations thereof. Greater than 2, 3, 4, 5, 6, 7, 8, 9, 10, 11, 12, 13, 14, 15, or 16 amino acids can be selected. Fewer than 20, 19, 18, 17, 16, 15, 14, 13, 12, 11, 10, 9, 8, 7, 6, 5, or 4 amino acids can be selected. Homologs can be sequence homologs or structural homologs. Appropriate methods of alignment to compare sequence or structure between homologous proteins or domains (homologs) can be used to identify homologous regions of structure. Homologs, if defined in terms of sequence identity, can be those having less than 98, 96, 94, 92, 90, 88, 86, 84, 82, 80, 78, 76, 74, 72, 70, 68, 66, 64, 62, 60, 58, 56, 54, 52, 50, 48, 46, 44, 42, 40, 38, 36, 34, 32, 30, 28, 26, 24, 22, or 20% sequence identity. Homologs, if defined in terms of sequence identity, can be those having greater than 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96 or 98% sequence identity.

The method can include the use of molecular dynamics calculations. These calculations can be used in providing or refining the model of the structure of the CAP-Gly domain, studying the interaction of at least one candidate ligand with the model, and in selecting a compound which is predicted to act as a ligand. Studying the interaction of a candidate ligand with the model and selecting a compound which is predicted to act as a ligand can also be accomplished using visual inspection of the provided model of the structure of the CAP-Gly domain, the structure of the compound and/or a combination of the two, including a model of the CAP-Gly domain with a bound or interacting compound molecule.

The method can include the use of assays to determine binding or absence of binding between the compound and a CAP-Gly domain. A CAP-Gly domain provided for this purpose can be a native protein containing a CAP-Gly domain, a fragment of a protein containing a CAP-Gly domain, or a polypeptide that includes a CAP-Gly domain. The protein, fragment or polypeptide can be purified or isolated. The assay can be an in vivo assay, an ex vivo assay, or an in vitro assay. One assay contemplated is an assay that monitors the assembly of a virus, particularly wherein the virus assembly assay monitors the assembly of a virus selected from the group consisting of large DNA viruses and recombinants and variants thereof. Examples of such viruses include, but are not limited to, poxviruses, iridoviruses, and African swine fever virus. Another assay contemplated is an assay that monitors chaperone activity. Examples of such assays are known to those of skill in the art and can be adapted to the present invention, if necessary, or used as they currently exist.

In a twenty-first aspect, the present invention relates to a method of screening compounds to identify ligands with biological effects. The method includes: contacting a polypeptide comprising a CAP-Gly domain with at least one compound; assaying for a selected biological effect; assaying for the)selected biological effect in the absence of the at least one compound; and comparing the level of the selected biological effect in the presence of the at least one compound to that in the absence of the at least one compound, whereby compounds are identified as ligands with biological effects when the level of the selected biological effect in the presence of the compound differs from the level of the selected biological effect in the absence of the compound. Compounds to be screened can be selected from chemical libraries, natural products libraries, and combinatorial libraries or from other collections of compounds. The biological effect to be monitored can include, but are not limited to, those that have an effect on microtubule formation, microtubule organization, viral capsid formation, virus factory formation, plaque formation, aggresome formation and chaperone activity. The assay can be an in vivo assay, an ex vivo assay, or an in vitro assay.

Web-links to pages on the WWW which are of particular use in describing the state of the art, at the time of this disclosure, can be found at:

http://www.soi.city.ac.uk/˜drg/seminars/protein-topology/;

http://www.soi.city.ac.uk/˜drg/seminars/nato_asi/; http://www.rcsb.org/pdb/; and links to other webpages contained therein. This statement is not a representation that any information contained therein is prior art in respect to the invention, nor that any material contained therein is material to patentability of the present invention. Furthermore, the representation is made only in respect to the information available when these pages were last reviewed and does not take into account in additions, deletions or alterations to the cited webpages that occurred prior to, concurrently with, or subsequent to review.

Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C or is at ambient temperature, and pressure is at or near atmospheric.

### EXAMPLE ONE

Cytoskeleton-associated proteins (CAPs) are involved in organization of cellular filaments and transportation of vesicles and organelles along the cytoskeleton network. A conserved motif, CAP-Gly, has been identified in a number of CAPs, including CLIP-170, kinesins and dyneins.

The crystal structure of the CAP-Gly domain of C. elegans F53F4.3 protein, solved by single-wavelength sulfur-anomalous phasing, revealed a novel protein fold containing three β-sheets. The most conserved sequence, GKNDG, is located in two consecutive sharp turns on the surface, forming the entrance to a hydrophobic groove. The groove residues are highly conserved as measured from the information content of the aligned sequences. The C-terminal tail of another molecule in the crystal is bound in this groove. Other interesting structural features are also identified in this commonly distributed domain in CAPs.

The cytoskeleton of a eukaryotic cell controls the spatial distribution and the movement of vesicles, organelles and large protein complexes. Much of the cytoskeleton is based upon three types of protein filaments: microtubules, intermediate filaments, and actin filaments. There are extensive interactions between the filaments mediated by a large number of cytoskeleton-associated proteins (Goode et al., “Functional cooperation between the microtubule and actin cytoskeletons” Current Opinioin in Cell Biology 12: 63-71 (2000); Houseweart et al., “Cytoskeletal linkers: New MAPs for old destinations” Dispatch 9: R864-R866 (1999)). Some of the well-studied cytoskeletal proteins, such as kinesins and dyneins, which act as motors, are bound directly to microtubules (Karki et al., “Cytoplasmic dynein and dynactin in cell division and intracellular transport” Current Opinion in Cell Biology 11: 45-53 (1999); Nogales, “Structural Insights Into Microtubule Function” Annual Reviews of Biophysics and Biomolecular Structures 30: 397-420 (2001)). For instance, microtubules and actin filaments are cross-linked by microtubule-associated proteins such as myosin-kinesin complexes, myosin-CLIP-170 complexes, and dynein-dynactin complexes (Goode et al., Curr. Opin. Cell Biol. 12: 63-71 (2000); Fuchs and Yang, Cell 98: 547-550 (1999). These protein molecules or molecular assemblies have one end that binds to the microtubule and another end that binds to different vesicles or organelles. Movements related to nucleus positioning or mitotic spindle orientation are orchestrated between microtubules and actin filaments through dynamic interactions of different cytoskeleton-associated proteins. The cytoskeleton is a dynamic network that disassembles and reassembles constantly according to functional requirements. Regulation of the dynamic assembly of the cytoskeleton and motor proteins involves other non-motor accessory proteins. The term “+TIPS” has recently been proposed to describe microtubule plus-end-tracking proteins (Schuyler and Pellman, Cell 105: 421-424 (2001)). This class of proteins tends to localize to the plus end of microtubules, and these proteins are the most conserved microtubule-associated proteins.

Common features have been identified for cytoskeleton-associated proteins. One common feature of a set of these proteins is the presence of the glycine-rich cytoskeleton-associated protein (CAP-Gly) domain. This characteristic domain was first discovered in restin (or CLIP-170), the prototype +TIP, and three other proteins by use of sequence homology analysis (Riehemann et al., “Sequence homologies between four cytoskeleton-associated proteins” Trends Biochem. Sci. 18: 82-83 (1993)). This domain is present in a Pfam family of 69 proteins (Bateman et al., Nucl. Acids Res. 30: 276-280 (2002) Restin is a filament-associated protein abundant in the tumoral cells characteristic for Hodgkin's disease (Bilbe et al., EMBO J. 11; 2103-2113 (1992)). Recently, this motif of three repeats was also found in the familial cylindromatosis tumor suppressor gene CKLD (Bignell et al., “Identification of the familial cylindromatosis tumor-suppressor gene” Nature Genetics 25: 160-165 (2000)). Most proteins contain only one CAP-Gly motif, whereas others may have two or three. The wide distribution of the CAP-Gly motif in cytoskeleton-associated proteins suggests that this domain could be a common adhesive domain for attachment to microtubules. However, the structure of the domain was unknown.

The CAP-Gly domain can also be found in some motor-associated proteins such as dynactin chain 1 (Collin et al., Genomics 53: 359-364 (1998)) and Drosophila kinesin-73 (Li et al., Proc. Natl. Acad. Sci. USA 94: 1086-1091 (1997)). The two CAP-Gly domains in CLIP-170 located at the N terminus were shown to mediate the binding of the CLIP-170 protein to the microtubules (Pierre, et al., Cell 70: 887-900 (1992)). The C-terminal region of CLIP-170 carries the domains for organelle binding suggesting that CLIP-170 is a microtubule-organelle linker protein (Scheel et al., J. Biol. Chem. 274: 25883-25891 (1999)). The CAP-Gly domains of CLIP-170 bind specifically to the growing (plus) end of the microtubule (Perez et al., Cell 96: 517-527 (1999)), whereas the opposite end of CLIP-170 mediates interactions with dynactin (Vaughan et al., J. Cell Sci. 112: 1437-1447 (1999)) or myosin (Lantz and Miller, J. Cell Biol. 140: 897-910 (1998)). This characteristic feature might allow CLIP proteins to participate in the dynamic control of cargo transport along microtubules or between microtubules and actin filaments.

By analysis of amino acid sequence, we find that the protein F53F4.3 from C. elegans is a tubulin-specific chaperone B. This is demonstrated by its homology to the human gene sequence found in the Entrez database provided by the National Center for Biotechnology Information (NCBI) using sequence gi3025329 (hypothetical protein F53F4.3 in chromosome V and sequence gi3023518 (tubulin-specific chaperone B, also known as tubulin folding cofactor B and cytoskeleton-associated protein CKAPI). Between the two sequences, identities were found for 99/230 residues (43%), positives for 139/230 residues (60%) and gaps for 9/230 residues (3%). Lopez-Fanaraga et al. discuss important aspects of tubulin-specific chaperone B in “Review: Postchaperonin Tubulin Folding Cofactors and their Role in Microtubule Dynamics,” J. Structural Biology, 135; 219-229 (2001).

Further indications regarding the function of CAP-Gly domain containing proteins can be found in the literature. In particular, Delabie et al., “Restin in Hodgkin's disease and anaplastic large cell lymphoma,” Leuk. Lymphoma 12: 21-26 (1993); Johnston et al., Cytoplastic dynein/dynactin mediates the assembly of aggresomes,“Cell Motil. Cytoskeleton 53(1): 26-38 (2002); and Gross et al., “Coordination of opposite-polarity microtubule motors,” J. Cell Biology 156(4); 715-724 (2002).

The role that CAP-Gly domains, when present in specific proteins, can play in the formation of aggresomes is of particular interest. Indicated as essential for the proper function of certain microtubule-associated proteins and cytoskeletal elements, alteration, ablation or restoration, in the case of proteins wherein normal function is lacking, of function conferred by CAP-Gly domains can be used to effect a number of disease states. For example, CAP-Gly domain function plays a role in the formation of cellular structures called aggresomes. These structures are formed in cells in response to an accumulation of misfolded protein. First identified as sites able to sequester misfolded cystic fibrosis transmembrane conductance receptors or presenilin, it is now thought that aggressomes can be a general cellular response (Johnston et al., Aggresomes; a cellular response to misfolded proteins,” J. Cell Biol. 143: 1883-1898 (1998); Wigley et al., “Dynamic association of proteasomal machinery with the centrosome,” J. Cell Biol. 145: 481-490 (1999); Garcia-Mata et al., “Characterization and dynamics of aggresome formation by a cytosolic GFP-chimera,” J. Cell Biol. 146: 1239-1254 (1999); Kopito, “Aggresomes, inclusion bodies and protein aggregration,” Trends Cell Biol. 10:524-530 (2000)). Cells normally use chaperones and proteases to remove misfolded proteins. However, if these mechanisms fail, or are functioning at too low a level relative to the amount of misfolded proteins present, potentially toxic protein aggregates can be transported along microtubules to an aggresome. Normally situated next to the microtubule organizing center of the cell, these aggresomes are typically sequestered from the rest of the cell. As indicated in the literature, dynein/dynactin, which comprise CAP-Gly domain and require CAP-Gly domain activity for proper function, act in the assembly of aggresomes (Johnston et al., Cytoplastic dynein/dynactin mediates the assembly of aggresomes,“Cell Motil. Cytoskeleton 53(1): 26-38 (2002)).

Diseases in which aggresome assembly has been implicated include, but are not limited to, Hodgkin's disease, anaplastic large cell lymphoma (Delabie et al., “Restin in Hodgkin's disease and anaplastic large cell lymphoma,” Leuk. Lymphoma 12: 21-26 (1993)) and neurodegenerative diseases such as, but not limited to alzheimer's disease (Johnston et al., Cytoplastic dynein/dynactin mediates the assembly of aggresomes,” Cell Motil. Cytoskeleton 53(1): 26-38 (2002)). Diseases wherein plaques, like those in alzheimer's disease, are formed can be mediated by the function of CAP-Gly domain containing proteins. Accordingly, modulation of the function and/or level of CAP-Gly domains can affect the rate at which plaques are formed and/or the rate diseases progress.

Aggresome assembly, and the cellular mechanisms for promoting aggresome assembly, have also been implicated in the assembly and replication of certain viruses. In particular, it has been known for many years that large DNA viruses such as poxviruses, iridoviruses and the closely related African Swine Fever (ASF) virus are assembled in discrete cytoplasmic structures. Often referred to as viral factories, these perinuclear structures contain viral DNA, high concentrations of structural proteins and the cellular membranes required for viral assembly. These structures also tend to exclude host proteins suggesting that the viruses can induce the formation of new subcellular structures to act as a scaffold for virus replication and assembly. The generation of specific assembly sites within cells could occur actively by targeting viral proteins into the subcellular structure or region.

Understanding the structural basis of critical proteins involved in these processes can allow screening of compounds to identify appropriate compounds for treatment of conditions such as, but not limited to, those described herein. As will be understood by those of skill in the art, the identification of the CAP-Gly domain's structure and its recognition as a discrete structural element allows the study of use of the domain and its structure to identify ligands that can bind to many different proteins that comprise the CAP-Gly domain. Accordingly, the present invention is not limited to the particular protein encoded by the F53F4.3, but includes, among other embodiments, the use of a structure according to that derived from F53F4.3 to design putative ligands, to identify ligands, and to use identified ligands to identify those that have significant biologic activity.

The benefits of the high throughput structural screening work described herein can be illustrated by the elucidation of a previously unknown protein fold. This new protein fold, along with others found as a result of structural genomics research increases the number of known, unique protein structures. A database of greater breadth of unique, characterized protein folds is a major goal of modern structural genomics research. A number of analyses have suggested that it would take between 16,000 to 50,000 protein structures to cover more than 90% of the protein fold space by comparative modeling (Vitkup et al., “Completeness in structural genomics” Nature Structural Biology 8: 559-566 (2001); Sali, “Target practice” Nature Structural Biology 8: 482-484 (2001)).

Here we report the first result from this high throughput (HTP) effort. The structure solution of the CAP-Gly domain of the C. elegans F53F4.3 protein provides a convincing example of how HTP X-ray crystallography can impact on our knowledge of new genes identified from genome sequencing.

The genome of C. elegans has been completely sequenced, and several proteins were predicted to have CAP-Gly domain based on homology. The genes from C. elegans were selected as targets by the Southeast Collaboratory for Structural Genomics (Norvell and Machalek, Nat. Struct. Biol. 7 (suppl.); 931-931 (2000)).

Based on the annotation of the C. elegans genome performed by the C. elegans sequencing consortium, primers were designed for cloning all predicted cDNAs into an ENTRY vector in the GATEWAY cloning system (Life Technologies) (The C.E.S.consortium, “Genome sequence of the nematode C. elegans: a platform for investigating biology” Science 282: 2012-2018 (1998); Walhout, A. J. et al., “GATEWAY recombinational cloning: application to the cloning of large numbers of open reading frames or ORFeomes” Methods Enzymol. 328: 575-592 (2000)). These ENTRY vectors were systematically recombined with the pDEST 17.1 expression vector in a 96-well format.

The expression of each recombinant protein expression vector was screened and vectors that expressed soluble proteins in E. coli were selected. F53F4.3 was one of the selected vectors that produced a high amount of soluble protein when protein expression in E. coli was induced at 18° C. The recombinant protein expressed by this vector has a hexahistidine tag and an eight amino acid peptide in front of the N-terminus and an eight amino acid peptide following the C-terminus of the gene. The His tag was included for purification by a nickel affinity column and both eight amino acid peptides at each terminus resulted from the particular recombination reaction used during the cloning process (Walhout et al., “GATEWAY recombinational cloning: application to the cloning of large numbers of open reading frames or ORFeomes” Methods Enzymol. 328: 575-592 (2000)). After nickel affinity FPLC gel filtration (S-75, Pharmacia), and ion exchange (Resource Q, Pharmacia) chromatography purification, the purified protein was concentrated to about 10 mg/ml for crystallization.

Before any crystals were produced, it was observed that the purified F53F4.3 protein, stored at 4° C., had cleaved in half. The two resulting fragments were isolated from the SDS PAGE gel and subjected to mass spectrometry analysis to determine the molecular weight of each fragment and to automated N-terminal amino acid sequencing to determine the N-terminal sequence of each fragment. It was determined that the C-terminal fragment corresponds to residues 101-292.

The nucleic acid sequence encoding residues 101-292 was subsequently cloned into a pET 28b vector (Novagen) for the purpose of expressing this protein fragment. The protein fragment was expressed with a hexahistidine tag and a thrombin cleavage site at the N-terminus of the fragment arranged such that treatment with thrombin generated the a polypeptide with amino acid sequence corresponding to amino acid residues 101-292. The protein was purified using the earlier-described protocol and was subjected to varying conditions to stimulate protein crystallization. One particular set of conditions resulted in large single crystals (˜0.5mm) in conjunction with crystallization by the hanging drop method (described by McPherson, “Crystallization of Biological Macromolecules” Cold Spring Harbor Laboratories Press, 1998). In particular, the hanging drop was made from one half volume purified protein solution and one half volume crystallization buffer. The purified protein solution in this example was of a concentration of 10 mg/ml. The crystallization buffer solution used in the reservoir during crystallization and to make up the hanging drop as described contained 1.8 M ammonium sulfate and 0.1 M MES (pH 6.5). Removal of the His-tag by thrombin cleavage did not appear to inhibit or enhance crystallization. While crystals in this particular example were formed from polypeptides that had been processed to remove the His-tag, polypeptides retaining the His-Tag can also be used. Using these conditions allows formation of suitable crystals in approximately four days. The temperature at which crystallization occurs is not critical. Crystallization was observed at 4° C. and at room temperature.

X-ray diffraction data from the crystals were collected to 2.5 Å resolution on a single crystal, flash-cooled to 100K. This work, done at beamline ID-17 (IMCA-CAT), Advanced Photon Source (APS) at the Argonne National Laboratory, was accomplished using a MarResearch 165 mm CCD detector and 1.74 Å X-rays following the protocol described previously (Liu et al., “Structure of the Ca²⁺-regulated photoprotein obelin at 1.7 Å resolution determined directly from its sulfur substructure” Protein Sci 9: 2085-2093 (2000)). The HKL2000 software package was used to determine a data collection strategy (99% completion) (Otwinowski et al., “Processing of X-ray Diffraction Data Collected in Oscillation Mode” Methods in Enzymology 276: 307-326 (1997)). Data collection was divided into 4 passes. The first and third passes covered one anomalous diffraction wedge (86°-146°), while the second and fourth passes covered the other anomalous diffraction wedge (266°-326°). The oscillation angle was 1° per frame for all the passes. Data processing was carried out using the HKL2000 software (Otwinowski et al., “Processing of X-ray Diffraction Data Collected in Oscillation Mode” Methods in Enzymology 276: 307-326 (1997)). Processed data from different passes were then merged. Statistics and particulars regarding the collection and processing of the data are given in Table 1.

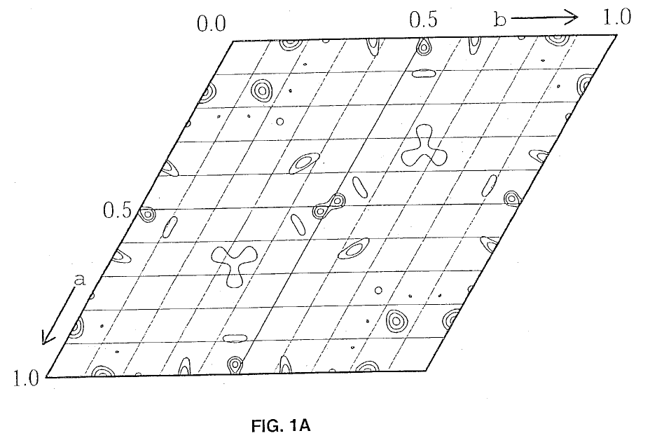

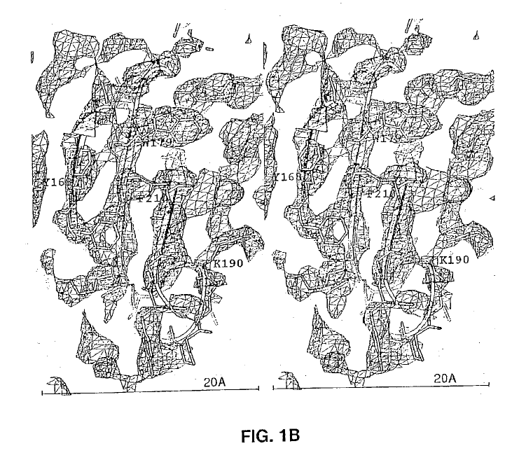

Low-resolution data (3.5 Å ) was used to locate the sulfur atom sites. A total of 1707 Bijvoet reflection pairs greater than 2σ, between 20.0 Å and 3.5 Å resolution (Bijvoet difference R-factor was 1.5%), were used to determine three sulfur atom positions (FIG. 1a) with SOLVE V1.16 (Terwilliger, “Multiwavelength anomalous diffraction phasing of macromolecular structures: analysis of MAD data as single isomorphous replacement with anomalous scattering data using the MADMRG Program” Methods Enzymol 276: 530-537 (1997)). These three sulfur sites could also have been easily determined by use of the program SHELXD (Sheldrick, “Phase annealing in SHELX-90: direct methods for larger structures” Acta Cryst. A46: 463-466 (1990)). Following determination of the three sulfur sites, PHASES (Furey et al., “PHASES-95: A Program Package for the Processing and Analysis of Diffraction Data from Macromolecules” Methods in Enzymology 277: 590-620 (1997)) was used to refine the three previously identified sulfur positions and to locate one additional sulfur site by Bijvoet difference Fourier analysis. In light of the final, solved structure, these heavy atom sites corresponded to sulfur atoms of two cysteines, to a sulfur atom of one methionine, and possibly to a Cl ion in the solvent region. For the purposes of the phase calculations, all heavy atom sites were treated as sulfur.

These four heavy atom sites were used to resolve between the enantiomorphic space groups P6122 and P6522. The handedness test feature in ISAS2001 was used at 20.0-3.5 Å resolution to identify P6122 as the correct space group because of the high figure-of-merit (FOM) and low map-inversion R-value (Wang, “Resolution of phase ambiguity in macromolecular crystallography” Methods Enzymol 115: 90-112 (1985)). The same four “sulfur” sites were used to estimate the protein phases at 3.0 Å resolution using ISAS (Wang, “Resolution of phase ambiguity in macromolecular crystallography” Methods Enzymol 115: 90-112 (1985)). After three filters and two cycles of phase extension, the final average figure-of-merit and map-inversion R-value were 0.73 and 0.281 respectively. The original electron density map is shown in FIG. 1b, in which the polypeptide chain could be traced for about 65 residues. After a polyAla model was fitted into the density, the original phases and the calculated phases from the polyAla model were provided to the wARP program with diffraction data between 50-1.8 Å collected on Raxis IV detector mounted on a Rigaku RU300 generator (Lamzin et al., “Current state of automated crystallographic data analysis” Nat Struct Biol 7, Suppl: 978-81 (2000)). On the basis of the wARP program's analysis, a new polyAla model with 95 residues was generated. Sequence assignment and refinement were carried out with programs O (Jones et al., “Improved methods for building protein models in electron density maps and the location of errors in these models” Acta Crystallogr A 47: 110-119 (1991)) and XPLOR3.5 (Brunger et al., “Crystallography & NMR system: A new software suite for macromolecular structure determination” Acta Crystallogr D Biol Crystallogr 54: 905-921 (1998)). The final model of 109 residues were refined against a data set to 1.77 Å resolution collected at APS (Table 2). The structure, as described in the table, possesses a number of interesting characteristic features.

Crystals of the wild type recombination F53F4.3 protein (residues 121-292) were grown in conditions found in a robotic screen using the 96-well sitting drop plates. the optimal reservoir solution consisted of 1.8 M ammonium sulfate and 0.1 M MES buffer at pH 6.5. The protein drop contained 5 microliters of the reservoir solution and 5 microliters of the protein solution at 10 mg/ml concentration in 20 mm HEPES buffer, pH 7.4. Crystals of 0.4×0.4×0.3 mm in size appeared in the drops in 4 days at room temperature. The space group was determined to be P6122 with a=b=61.16 Å, c=101.95 Å from x-ray diffraction data collected on a Raxis-IV image plate detector (MSC). To obtain phases for solving the crystal structure, the ISAS method was employed (Wang, Methods Enzymol. 115: 90-112 (1985)). It has been argued that the anomalous signal from the sulfur atoms present in the wild type proteins would be sufficient for phase determination using only single wavelength x-ray data (Liu et al., Protein Sci. 9: 2085-2093 (2000)). To collect x-ray diffraction data with high accuracy required for the ISAS method, crystals of F53F4.3 were cryo-frozen at 100 K and x-ray data were collected the wavelength of 1.74 Å to increase the anomalous signal from sulfur atoms while minimizing crystal damage. After screening a number of crystals a data set to 2.5 Å resolution was completed from the best crystal. This data set to the moderate resolution limit was collected for phasing only. Three sulfur atom positions could be located by the SOLVE program using SAS data. The Patterson peaks corresponding to the sulfur sites were clearly visible (FIG. 1A). A fourth site was found in further refinement. Phases derived from these anomalous sites were refined and extended by solvent flattening (Wang, Methods Enzymol. 115: 90-112 (1985)). An electron density map was calculated, and polypeptide chain tracing and some side chain identification were possible (FIG. 1B). A complete structure model corresponding to residues 135-229 was built in steps and refined to 1.77 Å using a high resolution data set and the X-plor program (Brunger, X-PLOR Version 3.1: A System for X-ray Crystallography and NMR, Yale University Press, New Haven and London (1992)). The structure refinement statistics are summarized in Table I.

The C-terminal domain of F53F4.3 is roughly spherical with a three-layer β/β structure (FIG. 4). The N-terminus has a nine residue α-helix (Asp135-Lys144) with relatively higher average temperature factors. This α-helix was preceded by 16 disordered residues that do make any stable contacts with the rest of this domain and could be available for inter-molecular interactions. The three-layer β/β structure contains seven anti-parallel β-strands. The first layer consists of β1, β2a, and β7. The second layer starts with the extension of β-strand 2 (β2b) and continues with β3 and β6. The last layer contains β4 and β5. The topology of the CAP-Gly domain could be defined as three antiparallel beta sheets of 3-3-2 strands. The two three-stranded sheets are L-shaped to each other with the last strand of the first sheet extended continuously to the first strand of the second sheet. The two-stranded sheet is on top of the second three-stranded sheet. The C-terminal six residues (Leu224 to Ile229) protrude out of the globular domain. The structure of this domain represents a new protein fold. In particular, the CAP-Gly domain appears to be a rigidly packed motif in the middle of two extended polypeptides. A search for three dimensional homology, using the method of Gibrat et al., found no similar structures from the non-redundant subset of the Protein Data Bank (Gibrat et al., “Surprising similarities in structure comparison” Current Opinion in Structural Biology 6: 377-385 (1996)).

The sequence of the F53F4.3 domain was provided to the EBI server (http://www.ebi.ac.uk/fasta33) to search the Swiss-Prot sequence database using the FASTA alignment method (Pearson, “Rapid and sensitive sequence comparison with FASTP and FASTA” Methods in Enzymology 183: 63-98 (1990)). The 58 aligned sequences, ranging from 55% to 22% identity, corresponded to the conserved cytoskeleton association protein glycine-rich motif (CAP-Gly) (Riehemann et al., “Sequence homologies between four cytoskeleton-associated proteins” Trends Biochem. Sci. 18: 82-83 (1993)); Watanabe et al., “Cloning, expression, and mapping of CKAPI, which encodes a putative cytoskeleton-associated protein containing a CAP-Gly domain ” Cytogenet Cell Genet 72: 208-211 (1996)). The Shannon information content was used as the measure of sequence conservation to determine the level of correspondence of the F53F4.3 domain to the CAP-Gly domain (Karlin et al., “Methods for assessing the statistical significance of molecular sequence features by using general scoring schemes” Proc. Natl. Acad. Sci. USA 87: 2264-2268 (1990)). This rigorous statistical procedure compares each position in the given alignment with the probability of randomly distributed amino acids weighted by the observed distribution of each amino acid in the C. elegans genome. A variety of structural alphabets may be employed in this analysis, for example, grouping the amino acids by charge or chemical class. The data presented here treat each of the 20 amino acids independently. A portion of the alignment and its annotation is shown in FIG. 3.

The longest stretch of highly conserved sequence observed corresponds to the portion of the sequence from Gly189 to Gly193. This sequence is located at the surface of the domain between β3 and β4. The five-residues, GKNDG, which comprise this segment are conserved in most homologous sequences. The few exceptions are those sequences where the asparagine (N) is substituted by a histidine (H) or the aspartic acid (D) is substituted by a serine (S). A loop structure opposite this conservative stretch, Val156 to Met160, was observed to have higher average temperature factors. This could be an indication that this part of the structure is flexible. The presence of this highly variable region among the many homologous sequences can indicate that the variable region is a characteristic recognition site for other protein interactions which are not conserved across the homologous members of the CAP-Gly family. It is contemplated that the conserved stretch can associate with the cytoskeleton. There is a conserved aromatic cluster including Tyr168, Phe174, Trp179, Tyr200, Phe201, and Phe210. The hydrophobic cluster may represent a stabilizing core structure. There is also a significant groove adjacent to the conserved 189-193 loop. The groove is lined by 12 residues: Val156, Gln 159, Arg162, Phe174, Trp179, Tyr184, Lys190, Asn191, Val195, Gly209, Phe210 and Val211. The sidechains of Phe174, Trp179, Phe210, along with Val195 form a large hydrophobic patch adjacent to the conserved 189-193 loop. A number of their main chain and side chain atoms are solvent-accessible in the crystal lattice, the extended C terminus of one molecule is packed into the groove of a symmetry-related molecule. In the crystal lattice, the extended C-terminus of one molecule can be packed into the hydrophobic groove of a symmetry-related molecule. The sidechain of residue Glu228 forms a buried salt bridge with Arg162 and a hydrogen bond with residue Tyr184. As was indicated in the sequence alignment, residue 162 is usually basic in character. It was also observed that hydrogen bonds can also form between mainchain atoms of residue Ile229 and the symmetry-related molecule. Lys-190 is also close to the acid C-terminus.

The CAP-Gly domains contain large numbers of conserved glycine residues. Two of these, Gly193 and Gly197, are involved in the formation of two consecutive sharp turns. The second of these sharp turns is a Type II β-turn. This unique double-turn motif exposes the most conserved region (GKNDG) in the CAP-Gly domain. The residue after the GKNDG sequence, often a serine/threonine, was observed to form a hydrogen bond with the sidechain of residue Asp192. Other conserved glycine residues in the CAP-Gly domain include: Gly170 and Gly177, which are involved in bending β2b and β3 which results in the loop connecting β2b and β3 to be perpendicular; Gly189, which is in the middle of an extended loop containing the most conserved stretch of sequence; Gly181, which is packed closely to the conserved Phe210 (these two residues are within 3.5 Å); and Gly208, which is packed closely to Phe201 (these two residues are within 4Å ). These conserved glycine residues can be required for maintaining the folding of the three-layer sheet.

The crystal structure of a widely distributed CAP-Gly domain is reported to high resolution. This domain was isolated from the C. elegans F53F4.3 protein when expressed in E. coli. Based on previous sequence alignment, the CAP-Gly domain was proposed to contain 52 amino acids (Pfam: PF1302) (Reihemann and Sorg, Trends Biochem. Sci. 18: 82-83 (1993)). These 52 amino acids constitute the conserved core region of this domain. However, the crystal structure revealed that this domain consists of 84 amino acids in this protein. One alpha helix and three beta-sheets form a novel protein fold not observed previously. Two conserved regions are identified on the surface based on information content analysis of the amino acid sequences from this protein family. A surface loop containing residues 193-197 (GKNDG) could present itself for interaction with the microtubule. The second feature is a groove that was occupied in our crystal structure by the C terminus of a neighboring molecule. A helix at the N terminus is loosely formed in this crystal structure and is located upstream from the CAP-Gly domain of C. elegans F53F4.3. The function of F53F4.3 is unknown in C. elegans.

The CAP-Gly domain was first identified by comparing restin, a filament-associated protein abundant in the tumoral cells characteristic for Hodgkin's disease ((Watanabe et al. “Cloning, expression, and mapping of CKAPI, which encodes a putative cytoskeleton-associated protein containing a CAP-GLY domain” Cytogenet Cell Genet 72: 208-211 (1996)), with other proteins known to be cytoskeleton associated proteins (Riehemann et al., “Sequence homologies between four cytoskeleton-associated proteins” Trends Biochem. Sci. 18: 82-83 (1993)). Recently, this motif of three repeats was also found in the familial cylindromatosis tumor-suppressor gene CYLD (Bignell et al., “Identification of the familial cylindromatosis tumor-suppressor gene” Nature Genetics 25: 160-165 (2000)). Most proteins identified as containing the CAP-Gly motif contain only a single copy of the motif. However, there are known proteins which contain two or three copies of the CAP-Gly motif. The wide distribution of the CAP-Gly motif in cytoskeleton-associated proteins including kinesins and dyneins indicates that it is involved in the attachment of these proteins to the cytoskeleton. The conserved patch identified on the surface is located centrally in the CAP-Gly domain. It is contemplated that this conserved patch offers a point of contact with the cytoskeleton. It is also contemplated that the hydrophobic groove that holds the C-terminus binds the filamentous proteins. For instance, the cytoplasmic linker protein CLIP-170 was shown to be require for the binding of endocytic vesicles to microtubules in vitro and to be co-localized with endocytic organelles in vivo (Pierre et al., Cell 70: 887-900 (1992)). There are two CAP-Gly domains in CLIP-170. When these two domains were deleted from CLIP-170, its binding to microtubules was diminished. The purified CLIP-170 forms an elongated dimer of a central coil-coil structure with its N-terminal domain binding to microtubules (Scheel et al., J. Biol. Chem. 274: 25883-25891(1999)). It is also contemplated that the (X-helix at the N-terminus of the CAP-Gly domain is involved in coil-coil formation as other cytoskeleton associated proteins (Riehemann et al., “Sequence homologies between four cytoskeleton-associated proteins” Trends Biochem. Sci. 18: 82-83 (1993)).

The precise pattern of the CAP-Gly domain interactions with the cytoskeleton remains to be delineated. There are three potential structural regions that could interact with the cytoskeleton. First, the conserved patch identified on the surface is central in the CAP-Gly domain, which could offer a point of contact with microtubules. The specially arranged glycine residues render an unusual structure feature protruding out on the protein surface. The highly conserved residues in this region could readily be available to fit into a receptive region on microtubules. Second, a groove that holds the C terminus of the neighboring molecules might also be a candidate for binding the filamentous proteins. In this structure, the ordered residues extend to the last residues of the C terminus. In other CAP-Gly-containing proteins, this domain is usually located in the middle of a long polypeptide chain. The C terminus could represent hypothetically the binding peptide from the cytoskeleton if such a peptide is required for binding. Third, the N terminus of the CAP-Gly domain might be involved in interactions with other cytoskeleton-associated proteins (Riehamann and Sorg, Trends Biochem. Sci. 18: 82-83 (1993)). The F53F4.3 does not form a dimer in vitro. However, the helix region of the CAP-Gly domain in other CAP-Gly proteins could be more extended and may contain residues that induce coil-coil interactions. Generally speaking, the coil-coil interaction of the CAP-Gly domain, if present, is likely to be with other cytoskeleton accessory proteins or to be involved in dimerization in CLIP-170 (Scheel et al., J. Biol. Chem. 274: 25883-25891 (1999)). The helix region has very high B-factor in this structure and is preceded by a long disordered region. It is possible that a helix would only be stable in the formation of a multiplex protein structure. The precise pattern of the CAP-Gly domain interactions with the cytoskeleton remains to be delineated. This new structure, however, could provide the platform for mapping these critical interactions.

The CAP-Gly domain could be viewed as a microtubule association module in cytoskeleton-associated proteins that may contain one, two, or three such domains with modularly increased affinity. The adhesive property of the CAP-Gly domain to microtubule is in another sense analogous to that of some DNA binding domains such as zinc fingers (Laity et al., Curr. Opin. Struct. Biol. 11; 39-46 (2001)). Both microtubules and DNA are biopolymers. The CAP-Gly domain or zinc fingers are protein modular units that are used for association to the polymer in a consecutive manner depending on the affinity requirement. The specificity for the binding location could be provided by sequence variations of the nonconserved regions or interacting with other regions. The CAP-Gly domain has also been found in dynein/dynactin (P150Glued) complexes. The conventional view is that the motor proteins could get on and off the microtubule by alternated motor domain binding to walk along the filament. The function of the CAP-Gly domain in this mobile complex may be to act as a tether to “glue” the complex on the microtubule. This will keep the traveling complex on the right track without totally falling off the microtubule.

This new structure provides the platform for mapping these critical interactions. Further, the present disclosure demonstrates that protein crystal structures can be determined in a high throughput manner using the naturally present sulfur sites as anomalous scatterers. Provided that high quality crystals could be grown, this process is relatively straightforward. It is also demonstrated that one prerequisite for growing high quality protein crystals is the production of highly purified, rigid, globular protein molecules. One method for obtaining such molecules is the subcloning of genes encoding the amino acid sequence of digested fragments from soluble protein preparations. The portion of a larger gene product which remains after partial digestion and/or degradation can be employed to identify the portion of a gene product that ultimately crystallizes to form desirable crystals. Use of this method, allows the elucidation of novel protein structure folds that correlated to significant biological functions without prior sequence-based target selection. This is particularly valuable since each protein family classified by sequence homology contains many members and it is difficult to predict which representative member can be used to yield useful crystals for structure determination. Random screening in a HTP manner can be a valuable alternative approach to complement other target selection strategies.

All publications, patents and patent applications cited in this specification are hereby incorporated by reference as if each individual publication, patent or patent application were specifically and individually indicated to be incorporated by reference.

Although the invention has been described in some detail by way of illustration and for the purposes of clarity and understanding, it will readily be apparent to one of ordinary skill in the art in light of the teachings regarding this invention that certain changes and modifications can be made thereto without departing from the spirit or scope of the appended claims.

TABLE 1
X-ray Crystallography Data Statistics

| a. Crystal | |
|---|---|
| Crystallization: | 1.8M (NH4)2SO4, 0.1M Mes, |
| | pH 6.5 |
| Space group: | P6122 |
| Cell parameters (Å): | a = 64.156 |
| | b = 64.156 |
| | c = 101.946 |
| b. Data collection and processing | |
| Data set used for phasing | |
| Wavelength (Å): | 1.74 |
| Resolution1 (Å): | 2.5 |
| Completeness (%): | 99.8 |
| Bijvoet Redundancy: | 16.9 |
| R_{merge}¹ (%): | 3.2 (6.3 in the last shell)² |
| Data set used for refinement | |
| Wavelength (Å): | 1.54 |
| Resolution (Å): | 1.77 |
| Reflections: | 12,651 |
| Completeness (%): | 99.2 (95.0 in the last shell) |
| R_{merge}¹ (%): | 4.3 (36.9 in the last shell) |
| Redundancy: | 9.8 |
| I/σ(I): | 19.0 (2.9 in the last shell) |
| c. Refinement statistics | |
| Reflections used in refinement: | 11,603 (at 50-1.77 Å) |
| Reflections used in R_{free}¹: | 622 (5%) |
| Residues modeled: | 135 to 229 |
| Water molecules incorporated: | 88 |
| R_{cryst}¹(%); | 22.5 |
| R_{free}¹: | 29.4 |
| Ramachandran plot: | 84.4% core region |
| | 15.6% allowed region |
| Average B-factors | |
| for protein: | 28.816 |
| for water: | 38.593 |
| Errors (not including glycine residues) | |
| Rmsd of bond length (Å): | 0.021 |
| Rmsd of bond angles (°): | 1.70 |

¹R_{merge} = ΣΣj|F − <F>|/Σ<F>.
²Highest resolution of data set followed by resolution range in highest bin for compiling statistics.

TABLE 2
Atomic Coordinates

| | | Amino | Amino | | | | | B |
|---|---|---|---|---|---|---|---|---|
| Atom | Atom | Acid | Acid | | | | | (Thermal |
| Number | Type | Type | Number | X | V | Z | Occupancy | Factor) |
| 1 | CB | SER | 135 | −11.350 | 20.947 | 56.966 | 1.00 | 81.94 |
| 2 | CG | SER | 135 | −12.713 | 20.547 | 56.919 | 1.00 | 83.92 |
| 3 | C | SER | 135 | −10.251 | 18.729 | 56.591 | 1.00 | 76.62 |
| 4 | O | SER | 135 | −11.100 | 17.847 | 56.459 | 1.00 | 78.43 |
| 5 | N | SER | 135 | −9.156 | 20.450 | 58.010 | 1.00 | 81.57 |
| 6 | CA | SER | 135 | −10.461 | 19.863 | 57.592 | 1.00 | 79.57 |
| 7 | N | ASP | 136 | −9.119 | 18.751 | 55.891 | 1.00 | 71.81 |
| 8 | CA | ASP | 136 | −8.825 | 17.722 | 54.900 | 1.00 | 66.92 |
| 9 | CB | ASP | 136 | −7.829 | 18.238 | 53.858 | 1.00 | 61.86 |
| 10 | CG | ASP | 136 | −8.290 | 17.972 | 52.434 | 1.00 | 60.91 |
| 11 | OD1 | ASP | 136 | −8.023 | 18.820 | 51.559 | 1.00 | 58.45 |
| 12 | OD2 | ASP | 136 | −8.923 | 16.919 | 52.189 | 1.00 | 58.99 |
| 13 | C | ASP | 136 | −8.281 | 16.447 | 55.522 | 1.00 | 65.46 |
| 14 | O | ASP | 136 | −7.650 | 16.472 | 56.578 | 1.00 | 64.24 |
| 15 | N | LYS | 137 | −8.548 | 15.330 | 54.857 | 1.00 | 62.86 |
| 16 | CA | LYS | 137 | −8.074 | 14.038 | 55.319 | 1.00 | 59.94 |
| 17 | CB | LYS | 137 | −8.858 | 12.909 | 54.640 | 1.00 | 63.47 |
| 18 | CG | LYS | 137 | −8.669 | 12.823 | 53.125 | 1.00 | 69.09 |
| 19 | CD | LYS | 137 | −9.779 | 13.556 | 52.367 | 1.00 | 72.44 |
| 20 | CE | LYS | 137 | −9.924 | 13.048 | 50.930 | 1.00 | 70.95 |
| 21 | NZ | LYS | 137 | −9.001 | 11.918 | 50.610 | 1.00 | 71.06 |
| 22 | C | LYS | 137 | −6.612 | 13.973 | S4.918 | 1.00 | 56.23 |
| 23 | O | LYS | 137 | −5.777 | 13.430 | 55.639 | 1.00 | 56.06 |
| 24 | N | LEU | 138 | −6.319 | 14.557 | 53.761 | 1.00 | 51.68 |
| 25 | CA | LEU | 138 | −4.970 | 14.590 | 53.217 | 1.00 | 49.61 |
| 26 | CB | LEU | 138 | −4.970 | 15.337 | 51.882 | 1.00 | 49.99 |
| 27 | CG | LEU | 138 | −5.790 | 14.681 | 50.765 | 1.00 | 52.38 |
| 28 | OD1 | LEU | 138 | −7.250 | 15.092 | 50.891 | 1.00 | 54.20 |
| 29 | CD2 | LEU | 138 | −5.240 | 15.090 | 49.407 | 1.00 | 51.01 |
| 30 | C | LEU | 138 | −3.994 | 15.249 | 54.185 | 1.00 | 46.29 |
| 31 | O | LEU | 138 | −2.902 | 14.734 | 54.426 | 1.00 | 45.20 |
| 32 | N | ASN | 139 | −4.389 | 16.389 | 54.739 | 1.00 | 43.15 |
| 33 | CA | ASN | 139 | −3.537 | 17.097 | 55.682 | 1.00 | 42.66 |
| 34 | CB | ASN | 139 | −4.136 | 18.460 | 56.016 | 1.00 | 38.37 |
| 35 | CG | ASN | 139 | −3.736 | 19.519 | 55.021 | 1.00 | 37.03 |
| 36 | OD1 | ASN | 139 | −2.574 | 19.601 | 54.627 | 1.00 | 35.80 |
| 37 | ND2 | ASN | 139 | −4.695 | 20.332 | 54.600 | 1.00 | 35.11 |
| 38 | C | ASN | 139 | −3.376 | 16.275 | 56.948 | 1.00 | 44.80 |
| 39 | O | ASN | 139 | −2.339 | 16.326 | 57.613 | 1.00 | 41.50 |
| 40 | N | GLU | 140 | −4.413 | 15.511 | 57.272 | 1.00 | 48.73 |
| 41 | CA | GLU | 140 | −4.406 | 14.663 | 58.456 | 1.00 | 53.43 |
| 42 | CB | GLU | 140 | −5.799 | 14.074 | 58.690 | 1.00 | 60.92 |
| 43 | CG | GLU | 140 | −6.847 | 15.090 | 59.123 | 1.00 | 71.76 |
| 44 | CD | GLU | 140 | −6.648 | 15.571 | 60.551 | 1.00 | 78.31 |
| 45 | OE1 | GLU | 140 | −7.414 | 16.459 | 60.989 | 1.00 | 82.39 |
| 46 | OE2 | GLU | 140 | −5.728 | 15.063 | 61.235 | 1.00 | 82.03 |
| 47 | C | GLU | 140 | −3.407 | 13.539 | 58.250 | 1.00 | 52.10 |
| 48 | O | GLU | 140 | −2.691 | 13.143 | 59.173 | 1.00 | 51.74 |
| 49 | N | GLU | 141 | −3.367 | 13.032 | 57.025 | 1.00 | 51.02 |
| 50 | CA | GLU | 141 | −2.461 | 11.953 | 56.677 | 1.00 | 52.17 |
| 51 | CB | GLU | 141 | −2.830 | 11.377 | 55.306 | 1.00 | 57.96 |
| 52 | CG | GLU | 141 | −3.813 | 10.215 | 55.354 | 1.00 | 67.31 |
| 53 | CD | GLU | 141 | −3.274 | 9.024 | 56.127 | 1.00 | 73.12 |
| 54 | OE1 | GLU | 141 | −2.406 | 8.304 | 55.583 | 1.00 | 77.30 |
| 55 | OE2 | GLU | 141 | −3.718 | 8.809 | 57.279 | 1.00 | 75.74 |
| 56 | C | GLU | 141 | −1.023 | 12.458 | 56.651 | 1.00 | 49.34 |
| 57 | O | GLU | 141 | −0.173 | 11.986 | 57.408 | 1.00 | 50.13 |
| 58 | N | ALA | 142 | −0.764 | 13.427 | 55.778 | 1.00 | 45.98 |
| 59 | CA | ALA | 142 | 0.567 | 14.004 | 55.624 | 1.00 | 42.24 |
| 60 | CB | ALA | 142 | 0.505 | 15.181 | 54.660 | 1.00 | 39.20 |
| 61 | C | ALA | 142 | 1.194 | 14.446 | 56.943 | 1.00 | 39.82 |
| 62 | O | ALA | 142 | 2.415 | 14.503 | 57.059 | 1.00 | 41.31 |
| 63 | N | ALA | 143 | 0.359 | 14.749 | 57.932 | 1.00 | 39.20 |
| 64 | CA | ALA | 143 | 0.833 | 15.209 | 59.234 | 1.00 | 38.19 |
| 65 | CB | ALA | 143 | −0.217 | 16.102 | 59.879 | 1.00 | 37.26 |
| 66 | C | ALA | 143 | 1.195 | 14.078 | 60.184 | 1.00 | 39.69 |
| 67 | O | ALA | 143 | 1.971 | 14.272 | 61.119 | 1.00 | 39.76 |
| 68 | N | LYS | 144 | 0.626 | 12.900 | 59.947 | 1.00 | 43.58 |
| 69 | CA | LYS | 144 | 0.880 | 11.725 | 60.784 | 1.00 | 45.47 |
| 70 | CB | LYS | 144 | 0.356 | 10.468 | 60.082 | 1.00 | 51.80 |
| 71 | CG | LYS | 144 | −0.876 | 9.852 | 60.729 | 1.00 | 61.43 |
| 72 | CD | LYS | 144 | −0.505 | 8.981 | 61.926 | 1.00 | 65.88 |
| 73 | CE | LYS | 144 | −1.412 | 9.263 | 63.117 | 1.00 | 67.63 |
| 74 | NZ | LYS | 144 | −1.389 | 10.701 | 63.507 | 1.00 | 67.97 |
| 75 | C | LYS | 144 | 2.354 | 11.516 | 61.143 | 1.00 | 41.21 |
| 76 | O | LYS | 144 | 2.694 | 11.290 | 62.305 | 1.00 | 38.15 |
| 77 | N | ASN | 145 | 3.222 | 11.602 | 60.141 | 1.00 | 39.14 |
| 78 | CA | ASN | 145 | 4.649 | 11.394 | 60.344 | 1.00 | 38.82 |
| 79 | CB | ASN | 145 | 5.204 | 10.544 | 59.206 | 1.00 | 44.45 |
| 80 | CG | ASN | 145 | 4.955 | 9.067 | 59.412 | 1.00 | 50.10 |
| 81 | OD1 | ASN | 145 | 4.427 | 8.388 | 58.532 | 1.00 | 54.99 |
| 82 | ND2 | ASN | 145 | 5.334 | 8.558 | 60.582 | 1.00 | 51.85 |
| 83 | C | ASN | 145 | 5.500 | 12.652 | 60.481 | 1.00 | 37.59 |
| 84 | O | ASN | 145 | 6.720 | 12.558 | 60.635 | 1.00 | 39.85 |
| 85 | N | iLE | 146 | 4.882 | 13.827 | 60.423 | 1.00 | 33.41 |
| 86 | CA | ILE | 146 | 5.655 | 15.056 | 60.547 | 1.00 | 30.29 |
| 87 | CB | ILE | 146 | 4.909 | 16.271 | 59.928 | 1.00 | 28.40 |
| 88 | CG2 | ILE | 146 | 5.755 | 17.542 | 60.090 | 1.00 | 25.16 |
| 89 | CG1 | ILE | 146 | 4.618 | 16.002 | 58.445 | 1.00 | 26.59 |
| 90 | CD1 | ILE | 146 | 3.942 | 17.151 | 57.716 | 1.00 | 28.39 |
| 91 | C | ILE | 146 | 5.952 | 15.326 | 62.015 | 1.00 | 29.18 |
| 92 | O | ILE | 146 | 5.043 | 15.560 | 62.808 | 1.00 | 29.76 |
| 93 | N | MET | 147 | 7.233 | 15.285 | 62.370 | 1.00 | 29.53 |
| 94 | CA | MET | 147 | 7.655 | 15.514 | 63.746 | 1.00 | 29.85 |
| 95 | CB | MET | 147 | 8.409 | 14.289 | 64.286 | 1.00 | 34.62 |
| 96 | CG | MET | 147 | 7.851 | 12.944 | 63.837 | 1.00 | 39.84 |
| 97 | SD | MET | 147 | 6.197 | 12.622 | 64.484 | 1.00 | 46.09 |
| 98 | CE | MET | 147 | 6.524 | 12.648 | 66.220 | 1.00 | 46.01 |
| 99 | C | MET | 147 | 8.540 | 16.747 | 63.899 | 1.00 | 28.66 |
| 100 | O | MET | 147 | 9.377 | 17.037 | 63.036 | 1.00 | 25.17 |
| 101 | N | VAL | 148 | 8.350 | 17.465 | 65.008 | 1.00 | 26.74 |
| 102 | CA | VAL | 148 | 9.142 | 18.650 | 65.302 | 1.00 | 24.60 |
| 103 | CB | VAL | 148 | 8.719 | 19.288 | 66.657 | 1.00 | 23.60 |
| 104 | CG1 | VAL | 148 | 9.685 | 20.399 | 67.052 | 1.00 | 17.06 |
| 105 | CG2 | VAL | 148 | 7.291 | 19.833 | 66.547 | 1.00 | 22.12 |
| 106 | C | VAL | 148 | 10.599 | 18.196 | 65.358 | 1.00 | 23.93 |
| 107 | O | VAL | 148 | 10.905 | 17.154 | 65.917 | 1.00 | 23.86 |
| 108 | N | GLY | 149 | 11.491 | 18.974 | 64.759 | 1.00 | 25.20 |
| 109 | CA | GLY | 149 | 12.893 | 18.609 | 64.744 | 1.00 | 25.61 |
| 110 | C | GLY | 149 | 13.293 | 18.011 | 63.406 | 1.00 | 25.59 |
| 111 | O | GLY | 149 | 14.464 | 18.038 | 63.036 | 1.00 | 26.72 |
| 112 | N | ASN | 150 | 12.316 | 17.477 | 62.679 | 1.00 | 25.30 |
| 113 | CA | ASN | 150 | 12.561 | 16.866 | 61.372 | 1.00 | 24.14 |
| 114 | CB | ASN | 150 | 11.271 | 16.236 | 60.810 | 1.00 | 23.55 |
| 115 | CG | ASN | 150 | 10.896 | 14.920 | 61.484 | 1.00 | 23.94 |
| 116 | OD1 | ASN | 150 | 11.669 | 14.353 | 62.255 | 1.00 | 33.09 |
| 117 | ND2 | ASN | 150 | 9.696 | 14.433 | 61.189 | 1.00 | 20.75 |
| 118 | C | ASN | 150 | 13.056 | 17.887 | 60.348 | 1.00 | 23.77 |
| 119 | O | ASN | 150 | 12.617 | 19.038 | 60.338 | 1.00 | 21.30 |
| 120 | N | ARG | 151 | 13.980 | 17.464 | 59.492 | 1.00 | 22.64 |
| 121 | CA | ARG | 151 | 14.448 | 18.337 | 58.429 | 1.00 | 20.29 |
| 122 | CB | ARG | 151 | 15.793 | 17.862 | 57.874 | 1.00 | 20.17 |
| 123 | CG | ARG | 151 | 16.996 | 18.286 | 58.718 | 1.00 | 22.19 |
| 124 | CD | ARG | 151 | 17.247 | 19.798 | 58.684 | 1.00 | 17.93 |
| 125 | NE | ARG | 151 | 17.504 | 20.285 | 57.331 | 1.00 | 17.25 |
| 126 | CZ | ARG | 151 | 17.729 | 21.559 | 57.021 | 1.00 | 17.19 |
| 127 | NH1 | ARG | 151 | 17.732 | 22.487 | 57.968 | 1.00 | 17.85 |
| 128 | NH2 | ARG | 151 | 17.939 | 21.912 | 55.759 | 1.00 | 19.58 |
| 129 | C | ARG | 151 | 13.340 | 18.185 | 57.383 | 1.00 | 19.92 |
| 130 | O | ARG | 151 | 12.681 | 17.142 | 57.310 | 1.00 | 18.29 |
| 131 | N | GYS | 152 | 13.124 | 19.211 | 56.573 | 1.00 | 19.98 |
| 132 | CA | GYS | 152 | 12.048 | 19.145 | 55.597 | 1.00 | 18.82 |
| 133 | CB | GYS | 152 | 10.732 | 19.548 | 56.265 | 1.00 | 19.19 |
| 134 | SG | GYS | 152 | 10.721 | 21.281 | 56.807 | 1.00 | 22.41 |
| 135 | C | GYS | 152 | 12.251 | 20.033 | 54.386 | 1.00 | 19.06 |
| 136 | O | GYS | 152 | 13.174 | 20.851 | 54.326 | 1.00 | 18.29 |
| 137 | N | GLU | 153 | 11.353 | 19.852 | 53.425 | 1.00 | 18.46 |
| 138 | CA | GLU | 153 | 11.342 | 20.625 | 52.202 | 1.00 | 20.68 |
| 139 | CB | GLU | 153 | 11.788 | 19.767 | 51.024 | 1.00 | 21.09 |
| 140 | CG | GLU | 153 | 11.997 | 20.565 | 49.762 | 1.00 | 28.39 |
| 141 | CD | GLU | 153 | 12.058 | 19.691 | 48.529 | 1.00 | 35.86 |
| 142 | OE1 | GLU | 153 | 11.188 | 18.800 | 48.385 | 1.00 | 40.46 |
| 143 | OE2 | GLU | 153 | 12.976 | 19.896 | 47.707 | 1.00 | 36.76 |
| 144 | C | GLU | 153 | 9.903 | 21.088 | 51.990 | 1.00 | 20.49 |
| 145 | O | GLU | 153 | 8.986 | 20.261 | 51.944 | 1.00 | 18.46 |
| 146 | N | VAL | 154 | 9.713 | 22.403 | 51.881 | 1.00 | 18.15 |
| 147 | CA | VAL | 154 | 8.390 | 22.984 | 51.683 | 1.00 | 17.58 |
| 148 | CB | VAL | 154 | 8.156 | 24.207 | 52.611 | 1.00 | 16.79 |
| 149 | CG1 | VAL | 154 | 6.788 | 24.822 | 52.335 | 1.00 | 15.64 |
| 150 | CG2 | VAL | 154 | 8.264 | 23.788 | 54.065 | 1.00 | 18.39 |
| 151 | C | VAL | 154 | 8.194 | 23.436 | 50.240 | 1.00 | 20.03 |
| 152 | O | VAL | 154 | 9.045 | 24.123 | 49.663 | 1.00 | 18.09 |
| 153 | N | THR | 155 | 7.064 | 23.038 | 49.664 | 1.00 | 21.68 |
| 154 | CA | THR | 155 | 6.720 | 23.403 | 48.297 | 1.00 | 26.92 |
| 155 | CB | THR | 155 | 6.852 | 22.199 | 47.343 | 1.00 | 28.59 |
| 156 | CG1 | THR | 155 | 6.054 | 21.113 | 47.834 | 1.00 | 34.53 |
| 157 | CG2 | THR | 155 | 8.291 | 21.746 | 47.247 | 1.00 | 27.24 |
| 158 | C | THR | 155 | 5.269 | 23.873 | 48.279 | 1.00 | 31.06 |
| 159 | O | THR | 155 | 4.360 | 23.067 | 48.093 | 1.00 | 32.72 |
| 160 | N | VAL | 156 | 5.045 | 25.170 | 48.475 | 1.00 | 34.35 |
| 161 | CA | VAL | 156 | 3.680 | 25.691 | 48.482 | 1.00 | 40.54 |
| 162 | CB | VAL | 156 | 3.454 | 26.675 | 49.677 | 1.00 | 39.54 |
| 163 | CG1 | VAL | 156 | 4.746 | 27.372 | 50.034 | 1.00 | 38.51 |
| 164 | CG2 | VAL | 156 | 2.373 | 27.693 | 49.334 | 1.00 | 39.92 |
| 165 | C | VAL | 156 | 3.315 | 26.371 | 47.161 | 1.00 | 43.40 |
| 166 | O | VAL | 156 | 3.851 | 27.424 | 46.818 | 1.00 | 45.66 |
| 167 | N | GLY | 157 | 2.403 | 25.748 | 46.422 | 1.00 | 45.95 |
| 168 | CA | GLY | 157 | 1.972 | 26.297 | 45.150 | 1.00 | 51.22 |
| 169 | C | GLY | 157 | 3.032 | 26.271 | 44.062 | 1.00 | 54.88 |
| 170 | O | GLY | 157 | 3.646 | 25.238 | 43.795 | 1.00 | 55.27 |
| 171 | N | ALA | 158 | 3.244 | 27.417 | 43.427 | 1.00 | 57.37 |
| 172 | CA | ALA | 158 | 4.230 | 27.532 | 42.361 | 1.00 | 59.35 |
| 173 | CB | ALA | 158 | 3.881 | 28.718 | 41.463 | 1.00 | 61.78 |
| 174 | C | ALA | 158 | 5.633 | 27.702 | 42.931 | 1.00 | 59.01 |
| 175 | O | ALA | 158 | 6.610 | 27.245 | 42.343 | 1.00 | 60.03 |
| 176 | N | GLN | 159 | 5.711 | 28.364 | 44.082 | 1.00 | 58.93 |
| 177 | CA | GLN | 159 | 6.965 | 28.640 | 44.781 | 1.00 | 57.77 |
| 178 | CB | GLN | 159 | 6.682 | 28.836 | 46.275 | 1.00 | 61.07 |
| 179 | CG | GLN | 159 | 6.480 | 30.282 | 46.705 | 1.00 | 66.57 |
| 180 | CD | GLN | 159 | 7.059 | 30.568 | 48.084 | 1.00 | 69.67 |
| 181 | OE1 | GLN | 159 | 8.174 | 30.149 | 48.401 | 1.00 | 71.34 |
| 182 | NE2 | GLN | 159 | 6.303 | 31.287 | 48.910 | 1.00 | 71.66 |
| 183 | C | GLN | 159 | 8.058 | 27.581 | 44.618 | 1.00 | 55.60 |
| 184 | O | GLN | 159 | 7.775 | 26.399 | 44.405 | 1.00 | 53.62 |
| 185 | N | MET | 160 | 9.309 | 28.027 | 44.732 | 1.00 | 52.33 |
| 186 | CA | MET | 160 | 10.483 | 27.159 | 44.633 | 1.00 | 48.60 |
| 187 | CB | MET | 160 | 11.733 | 27.999 | 44.369 | 1.00 | 53.25 |
| 188 | CG | MET | 160 | 13.004 | 27.191 | 44.220 | 1.00 | 62.45 |
| 189 | SD | MET | 160 | 13.131 | 26.437 | 42.593 | 1.00 | 70.70 |
| 190 | CE | MET | 160 | 12.379 | 27.727 | 41.567 | 1.00 | 71.92 |
| 191 | C | MET | 160 | 10.663 | 26.408 | 45.952 | 1.00 | 42.61 |
| 192 | O | MET | 160 | 10.523 | 26.997 | 47.020 | 1.00 | 42.25 |
| 193 | N | ALA | 161 | 10.979 | 25.119 | 45.879 | 1.00 | 33.59 |
| 194 | CA | ALA | 161 | 11.171 | 24.313 | 47.082 | 1.00 | 28.66 |
| 195 | CB | ALA | 161 | 11.688 | 22.937 | 46.705 | 1.00 | 29.00 |
| 196 | C | ALA | 161 | 12.130 | 24.972 | 48.071 | 1.00 | 25.04 |
| 197 | O | ALA | 161 | 13.192 | 25.456 | 47.688 | 1.00 | 25.71 |
| 198 | N | ARG | 162 | 11.749 | 24.993 | 49.344 | 1.00 | 20.83 |
| 199 | CA | ARG | 162 | 12.587 | 25.589 | 50.381 | 1.00 | 19.18 |
| 200 | CB | ARG | 162 | 11.948 | 26.874 | 50.912 | 1.00 | 21.09 |
| 201 | CG | ARG | 162 | 11.999 | 28.028 | 49.937 | 1.00 | 20.81 |
| 202 | CD | ARG | 162 | 10.904 | 29.037 | 50.243 | 1.00 | 26.33 |
| 203 | NE | ARG | 162 | 9.581 | 28.423 | 50.249 | 1.00 | 27.99 |
| 204 | CZ | ARG | 162 | 8.532 | 28.907 | 50.907 | 1.00 | 28.22 |
| 205 | NH1 | ARG | 162 | 8.637 | 30.021 | 51.623 | 1.00 | 27.61 |
| 206 | NH2 | ARG | 162 | 7.375 | 28.271 | 50.857 | 1.00 | 29.81 |
| 207 | C | ARG | 162 | 12.772 | 24.599 | 51.521 | 1.00 | 18.82 |
| 208 | O | ARG | 162 | 11.816 | 23.972 | 51.962 | 1.00 | 19.42 |
| 209 | N | ARG | 163 | 14.003 | 24.458 | 51.996 | 1.00 | 18.06 |
| 210 | CA | ARG | 163 | 14.290 | 23.525 | 53.075 | 1.00 | 18.46 |
| 211 | CB | ARG | 163 | 15.549 | 22.714 | 52.755 | 1.00 | 17.60 |
| 212 | CG | ARG | 163 | 15.421 | 21.857 | 51.521 | 1.00 | 14.67 |
| 213 | CD | ARG | 163 | 16.672 | 21.031 | 51.282 | 1.00 | 17.18 |
| 214 | NE | ARG | 163 | 16.607 | 20.378 | 49.980 | 1.00 | 18.92 |
| 215 | CZ | ARG | 163 | 17.553 | 19.595 | 49.475 | 1.00 | 18.30 |
| 216 | NH1 | ARG | 163 | 18.663 | 19.352 | 50.162 | 1.00 | 19.12 |
| 217 | NH2 | ARG | 163 | 17.384 | 19.056 | 48.276 | 1.00 | 19.89 |
| 218 | C | ARG | 163 | 14.472 | 24.231 | 54.407 | 1.00 | 17.45 |
| 219 | O | ARG | 163 | 14.776 | 25.420 | 54.463 | 1.00 | 18.90 |
| 220 | N | GLY | 164 | 14.289 | 23.473 | 55.479 | 1.00 | 19.03 |
| 221 | CA | GLY | 164 | 14.433 | 24.013 | 56.814 | 1.00 | 20.57 |
| 222 | C | GLY | 164 | 14.156 | 22.907 | 57.810 | 1.00 | 22.63 |
| 223 | O | GLY | 164 | 14.317 | 21.730 | 57.491 | 1.00 | 21.61 |
| 224 | N | GLU | 165 | 13.730 | 23.281 | 59.010 | 1.00 | 23.77 |
| 225 | CA | GLU | 165 | 13.433 | 22.319 | 60.062 | 1.00 | 23.10 |
| 226 | CB | GLU | 165 | 14.469 | 22.443 | 61.176 | 1.00 | 26.13 |
| 227 | CG | GLU | 165 | 14.187 | 21.565 | 62.380 | 1.00 | 35.98 |
| 228 | CD | GLU | 165 | 15.153 | 21.833 | 63.523 | 1.00 | 44.59 |
| 229 | OE1 | GLU | 165 | 16.131 | 22.585 | 63.306 | 1.00 | 46.79 |
| 230 | OE2 | GLU | 165 | 14.938 | 21.295 | 64.636 | 1.00 | 45.57 |
| 231 | C | GLU | 165 | 12.036 | 22.554 | 60.629 | 1.00 | 21.92 |
| 232 | O | GLU | 165 | 11.600 | 23.696 | 60.794 | 1.00 | 21.42 |
| 233 | N | VAL | 166 | 11.331 | 21.467 | 60.915 | 1.00 | 19.47 |
| 234 | CA | VAL | 166 | 9.987 | 21.560 | 61.471 | 1.00 | 20.47 |
| 235 | CB | VAL | 166 | 9.306 | 20.173 | 61.497 | 1.00 | 19.82 |
| 236 | CG1 | VAL | 166 | 7.911 | 20.280 | 62.084 | 1.00 | 19.78 |
| 237 | CG2 | VAL | 166 | 9.247 | 19.603 | 60.091 | 1.00 | 17.14 |
| 238 | C | VAL | 166 | 10.087 | 22.104 | 62.895 | 1.00 | 21.45 |
| 239 | O | VAL | 166 | 10.777 | 21.527 | 63.739 | 1.00 | 21.59 |
| 240 | N | ALA | 167 | 9.416 | 23.222 | 63.162 | 1.00 | 19.16 |
| 241 | CA | ALA | 167 | 9.463 | 23.807 | 64.497 | 1.00 | 19.84 |
| 242 | CB | ALA | 167 | 9.903 | 25.261 | 64.412 | 1.00 | 18.14 |
| 243 | C | ALA | 167 | 8.127 | 23.709 | 65.220 | 1.00 | 21.01 |
| 244 | O | ALA | 167 | 8.048 | 23.960 | 66.422 | 1.00 | 22.24 |
| 245 | N | TYR | 168 | 7.082 | 23.323 | 64.492 | 1.00 | 19.77 |
| 246 | CA | TYR | 168 | 5.748 | 23.230 | 65.072 | 1.00 | 21.00 |
| 247 | CB | TYR | 168 | 5.167 | 24.651 | 65.217 | 1.00 | 22.16 |
| 248 | CG | TYR | 168 | 3.854 | 24.751 | 65.975 | 1.00 | 22.88 |
| 249 | CD1 | TYR | 168 | 3.828 | 25.138 | 67.320 | 1.00 | 24.93 |
| 250 | CE1 | TYR | 168 | 2.628 | 25.250 | 68.016 | 1.00 | 25.25 |
| 251 | CD2 | TYR | 168 | 2.639 | 24.478 | 65.347 | 1.00 | 22.45 |
| 252 | CE2 | TYR | 168 | 1.432 | 24.586 | 66.032 | 1.00 | 23.58 |
| 253 | CZ | TYR | 168 | 1.434 | 24.973 | 67.368 | 1.00 | 27.36 |
| 254 | OH | TYR | 168 | 0.241 | 25.091 | 68.050 | 1.00 | 31.33 |
| 255 | C | TYR | 168 | 4.814 | 22.379 | 64.213 | 1.00 | 21.93 |
| 256 | O | TYR | 168 | 4.856 | 22.449 | 62.984 | 1.00 | 22.45 |
| 257 | N | VAL | 169 | 3.979 | 21.569 | 64.864 | 1.00 | 21.73 |
| 258 | CA | VAL | 169 | 3.003 | 20.738 | 64.156 | 1.00 | 24.21 |
| 259 | CB | VAL | 169 | 3.478 | 19.279 | 63.970 | 1.00 | 22.52 |
| 260 | CG1 | VAL | 169 | 2.612 | 18.606 | 62.925 | 1.00 | 23.29 |
| 261 | CG2 | VAL | 169 | 4.929 | 19.234 | 63.560 | 1.00 | 24.20 |
| 262 | C | VAL | 169 | 1.702 | 20.697 | 64.951 | 1.00 | 26.09 |
| 263 | O | VAL | 169 | 1.709 | 20.355 | 66.137 | 1.00 | 30.33 |
| 264 | N | GLY | 170 | 0.591 | 21.051 | 64.307 | 1.00 | 25.69 |
| 265 | CA | GLY | 170 | −0.691 | 21.031 | 64.997 | 1.00 | 24.81 |
| 266 | C | GLY | 170 | −1.614 | 22.210 | 64.736 | 1.00 | 25.46 |
| 267 | O | GLY | 170 | −1.424 | 22.975 | 63.790 | 1.00 | 24.23 |
| 268 | N | ALA | 171 | −2.623 | 22.353 | 65.590 | 1.00 | 24.23 |
| 269 | CA | ALA | 171 | −3.601 | 23.428 | 65.469 | 1.00 | 22.30 |
| 270 | CB | ALA | 171 | −4.847 | 23.084 | 66.293 | 1.00 | 19.67 |
| 271 | G | ALA | 171 | −3.035 | 24.778 | 65.916 | 1.00 | 19.59 |
| 272 | O | ALA | 171 | −2.101 | 24.842 | 66.717 | 1.00 | 18.47 |
| 273 | N | THR | 172 | −3.605 | 25.854 | 65.384 | 1.00 | 20.07 |
| 274 | CA | THR | 172 | −3.181 | 27.200 | 65.746 | 1.00 | 20.35 |
| 275 | CB | IHR | 172 | −2.338 | 27.860 | 64.628 | 1.00 | 17.19 |
| 276 | CG1 | THR | 172 | −3.163 | 28.067 | 63.479 | 1.00 | 16.39 |
| 277 | CG2 | THR | 172 | −1.152 | 26.980 | 64.248 | 1.00 | 16.60 |
| 278 | G | THR | 172 | −4.416 | 28.065 | 65.998 | 1.00 | 22.32 |
| 279 | O | THR | 172 | −5.554 | 27.586 | 65.918 | 1.00 | 24.63 |
| 280 | N | LYS | 173 | −4.189 | 29.342 | 66.291 | 1.00 | 22.52 |
| 281 | CA | LYS | 173 | −5.277 | 30.271 | 66.554 | 1.00 | 19.40 |
| 282 | CB | LYS | 173 | −4.855 | 31.286 | 67.620 | 1.00 | 18.64 |
| 283 | CG | LYS | 173 | −4.576 | 30.680 | 68.986 | 1.00 | 18.54 |
| 284 | CD | LYS | 173 | −4.137 | 31.741 | 69.990 | 1.00 | 20.93 |
| 285 | CE | LYS | 173 | −5.216 | 32.801 | 70.216 | 1.00 | 20.92 |
| 286 | NZ | LYS | 173 | −6.350 | 32.322 | 71.056 | 1.00 | 22.87 |
| 287 | C | LYS | 173 | −5.757 | 31.029 | 65.319 | 1.00 | 19.29 |
| 288 | O | LYS | 173 | −6.892 | 31.512 | 65.297 | 1.00 | 20.02 |
| 289 | N | PHE | 174 | −4.916 | 31.132 | 64.291 | 1.00 | 16.76 |
| 290 | CA | PHE | 174 | −5.305 | 31.889 | 63.109 | 1.00 | 17.45 |
| 291 | CB | PHE | 174 | −4.069 | 32.447 | 62.375 | 1.00 | 14.18 |
| 292 | CG | PHE | 174 | −3.063 | 31.411 | 61.991 | 1.00 | 15.23 |
| 293 | CD1 | PHE | 174 | −3.199 | 30.703 | 60.802 | 1.00 | 14.97 |
| 294 | CD2 | PHE | 174 | −1.963 | 31.154 | 62.810 | 1.00 | 16.28 |
| 295 | CE1 | PHE | 174 | −2.255 | 29.754 | 60.427 | 1.00 | 14.52 |
| 296 | CE2 | PHE | 174 | −1.011 | 30.209 | 62.449 | 1.00 | 14.91 |
| 297 | CZ | PHE | 174 | −1.157 | 29.507 | 61.254 | 1.00 | 16.18 |
| 298 | C | PHE | 174 | −6.217 | 31.157 | 62.136 | 1.00 | 20.39 |
| 299 | O | PHE | 174 | −6.996 | 31.793 | 61.426 | 1.00 | 21.83 |
| 300 | N | LYS | 175 | −6.120 | 29.832 | 62.088 | 1.00 | 18.45 |
| 301 | CA | LYS | 175 | −6.986 | 29.048 | 61.215 | 1.00 | 20.70 |
| 302 | CB | LYS | 175 | −6.547 | 29.131 | 59.748 | 1.00 | 18.01 |
| 303 | CG | LYS | 175 | −7.662 | 28.692 | 58.805 | 1.00 | 17.46 |
| 304 | CD | LYS | 175 | −7.384 | 28.995 | 57.347 | 1.00 | 17.69 |
| 305 | CE | LYS | 175 | −8.447 | 28.359 | 56.458 | 1.00 | 19.36 |
| 306 | NZ | LYS | 175 | −8.408 | 28.842 | 55.043 | 1.00 | 21.36 |
| 307 | C | LYS | 175 | −7.085 | 27.586 | 61.644 | 1.00 | 22.25 |
| 308 | O | LYS | 175 | −6.129 | 26.986 | 62.144 | 1.00 | 23.79 |
| 309 | N | GLU | 176 | −8.272 | 27.024 | 61.455 | 1.00 | 24.28 |
| 310 | CA | GLU | 176 | −8.533 | 25.646 | 61.826 | 1.00 | 26.30 |
| 311 | CB | GLU | 176 | −10.022 | 25.333 | 61.638 | 1.00 | 31.20 |
| 312 | CG | GLU | 176 | −10.500 | 25.385 | 60.193 | 1.00 | 38.78 |
| 313 | CD | GLU | 176 | −10.747 | 26.805 | 59.680 | 1.00 | 48.47 |
| 314 | OE1 | GLU | 176 | −10.944 | 27.735 | 60.503 | 1.00 | 44.41 |
| 315 | OE2 | GLU | 176 | −10.746 | 26.986 | 58.438 | 1.00 | 50.81 |
| 316 | C | GLU | 176 | −7.690 | 24.674 | 61.014 | 1.00 | 23.98 |
| 317 | O | GLU | 176 | −7.092 | 25.040 | 59.999 | 1.00 | 21.35 |
| 318 | N | GLY | 177 | −7.650 | 23.430 | 61.476 | 1.00 | 22.36 |
| 319 | CA | GLY | 177 | −6.893 | 22.406 | 60.788 | 1.00 | 23.02 |
| 320 | C | GLY | 177 | −5.487 | 22.281 | 61.326 | 1.00 | 22.72 |
| 321 | O | GLY | 177 | −5.132 | 22.921 | 62.316 | 1.00 | 23.70 |
| 322 | N | VAL | 178 | −4.685 | 21.451 | 60.666 | 1.00 | 23.17 |
| 323 | CA | VAL | 178 | −3.304 | 21.240 | 61.074 | 1.00 | 22.19 |
| 324 | CB | VAL | 178 | −2.869 | 19.778 | 60.818 | 1.00 | 21.26 |
| 325 | CG1 | VAL | 178 | −1.498 | 19.529 | 61.427 | 1.00 | 20.87 |
| 326 | CG2 | VAL | 178 | −3.889 | 18.829 | 61.417 | 1.00 | 21.75 |
| 327 | C | VAL | 178 | −2.376 | 22.184 | 60.308 | 1.00 | 19.37 |
| 328 | O | VAL | 178 | −2.574 | 22.437 | 59.117 | 1.00 | 20.31 |
| 329 | N | TRP | 179 | −1.375 | 22.710 | 61.007 | 1.00 | 19.13 |
| 330 | CA | TRP | 179 | −0.404 | 23.621 | 60.416 | 1.00 | 17.97 |
| 331 | CB | TRP | 179 | −0.604 | 25.046 | 60.954 | 1.00 | 17.35 |
| 332 | CG | TRP | 179 | −1.832 | 25.697 | 60.416 | 1.00 | 18.30 |
| 333 | CD2 | TRP | 179 | −1.993 | 26.260 | 59.113 | 1.00 | 17.53 |
| 334 | CE2 | TRP | 179 | −3.342 | 26.657 | 58.993 | 1.00 | 18.03 |
| 335 | CE3 | TRP | 179 | −1.126 | 26.466 | 58.028 | 1.00 | 15.51 |
| 336 | CD1 | TRP | 179 | −3.056 | 25.779 | 61.024 | 1.00 | 21.02 |
| 337 | NE1 | TRP | 179 | −3.968 | 26.352 | 60.174 | 1.00 | 18.79 |
| 338 | CZ2 | TRP | 179 | −3.846 | 27.248 | 57.829 | 1.00 | 19.14 |
| 339 | CZ3 | TRP | 179 | −1.622 | 27.049 | 56.880 | 1.00 | 15.12 |
| 340 | CH2 | TRP | 179 | −2.973 | 27.435 | 56.786 | 1.00 | 18.83 |
| 341 | C | TRP | 179 | 0.991 | 23.148 | 60.770 | 1.00 | 17.55 |
| 342 | O | TRP | 179 | 1.215 | 22.610 | 61.849 | 1.00 | 19.98 |
| 343 | N | VAL | 180 | 1.928 | 23.332 | 59.849 | 1.00 | 19.11 |
| 344 | CA | VAL | 180 | 3.316 | 22.957 | 60.098 | 1.00 | 19.55 |
| 345 | CB | VAL | 180 | 3.893 | 22.040 | 58.989 | 1.00 | 16.65 |
| 346 | CG1 | VAL | 180 | 5.308 | 21.625 | 59.352 | 1.00 | 15.42 |
| 347 | CG2 | VAL | 180 | 3.009 | 20.821 | 58.796 | 1.00 | 18.58 |
| 348 | C | VAL | 180 | 4.131 | 24.247 | 60.111 | 1.00 | 18.40 |
| 349 | O | VAL | 180 | 4.181 | 24.965 | 59.114 | 1.00 | 17.93 |
| 350 | N | GLY | 181 | 4.740 | 24.553 | 61.250 | 1.00 | 17.86 |
| 351 | CA | GLY | 181 | 5.562 | 25.744 | 61.340 | 1.00 | 15.58 |
| 352 | C | GLY | 181 | 6.957 | 25.312 | 60.956 | 1.00 | 17.26 |
| 353 | O | GLY | 181 | 7.466 | 24.323 | 61.483 | 1.00 | 19.39 |
| 354 | N | VAL | 182 | 7.581 | 26.037 | 60.039 | 1.00 | 17.32 |
| 355 | CA | VAL | 182 | 8.920 | 25.684 | 59.588 | 1.00 | 17.75 |
| 356 | CB | VAL | 162 | 8.916 | 25.345 | 58.074 | 1.00 | 17.69 |
| 357 | CG1 | VAL | 182 | 10.350 | 25.173 | 57.558 | 1.00 | 18.83 |
| 358 | CG2 | VAL | 182 | 8.099 | 24.080 | 57.831 | 1.00 | 15.00 |
| 359 | C | VAL | 182 | 9.942 | 26.791 | 59.820 | 1.00 | 20.30 |
| 360 | O | VAL | 182 | 9.652 | 27.974 | 59.615 | 1.00 | 18.52 |
| 361 | N | LYS | 183 | 11.134 | 26.390 | 60.261 | 1.00 | 20.12 |
| 362 | CA | LYS | 183 | 12.249 | 27.312 | 60.480 | 1.00 | 20.21 |
| 363 | CB | LYS | 183 | 13.019 | 26.921 | 61.744 | 1.00 | 23.26 |
| 364 | CG | LYS | 183 | 13.812 | 28.054 | 62.388 | 1.00 | 33.61 |
| 365 | CD | LYS | 183 | 14.468 | 27.614 | 63.706 | 1.00 | 37.23 |
| 366 | CE | LYS | 183 | 13.426 | 27.122 | 64.732 | 1.00 | 47.43 |
| 367 | NZ | LYS | 183 | 13.859 | 27.196 | 66.178 | 1.00 | 46.17 |
| 368 | C | LYS | 183 | 13.125 | 27.107 | 59.242 | 1.00 | 19.10 |
| 369 | O | LYS | 183 | 13.824 | 26.098 | 59.141 | 1.00 | 17.48 |
| 370 | N | TYR | 184 | 13.058 | 28.046 | 58.298 | 1.00 | 16.97 |
| 371 | CA | TYR | 184 | 13.809 | 27.966 | 57.039 | 1.00 | 17.47 |
| 372 | CB | TYR | 184 | 13.247 | 28.962 | 56.019 | 1.00 | 16.79 |
| 373 | CG | TYR | 184 | 11.873 | 28.608 | 55.509 | 1.00 | 17.50 |
| 374 | OD1 | TYR | 184 | 10.756 | 29.345 | 55.895 | 1.00 | 19.74 |
| 375 | CE1 | TYR | 184 | 9.476 | 29.013 | 55.437 | 1.00 | 21.46 |
| 376 | CD2 | TYR | 184 | 11.684 | 27.526 | 54.650 | 1.00 | 18.53 |
| 377 | CE2 | TYR | 184 | 10.414 | 27.185 | 54.186 | 1.00 | 20.04 |
| 378 | CZ | TYR | 184 | 9.314 | 27.933 | 54.584 | 1.00 | 21.82 |
| 379 | OH | TYR | 184 | 8.055 | 27.607 | 54.124 | 1.00 | 21.94 |
| 380 | C | TYR | 184 | 15.309 | 28.201 | 57.160 | 1.00 | 17.04 |
| 381 | O | TYR | 184 | 15.770 | 28.861 | 58.083 | 1.00 | 16.25 |
| 382 | N | ASP | 185 | 16.059 | 27.658 | 56.204 | 1.00 | 19.02 |
| 383 | CA | ASP | 185 | 17.507 | 27.812 | 56.176 | 1.00 | 20.81 |
| 384 | CB | ASP | 185 | 18.132 | 26.828 | 55.178 | 1.00 | 19.78 |
| 385 | CG | ASP | 185 | 18.116 | 25.402 | 55.677 | 1.00 | 17.83 |
| 386 | OD1 | ASP | 185 | 17.983 | 25.198 | 56.897 | 1.00 | 20.84 |
| 387 | OD2 | ASP | 185 | 18.234 | 24.478 | 54.848 | 1.00 | 21.41 |
| 388 | C | ASP | 185 | 17.840 | 29.243 | 55.766 | 1.00 | 21.19 |
| 389 | O | ASP | 185 | 18.794 | 29.836 | 56.263 | 1.00 | 24.03 |
| 390 | N | GLU | 186 | 17.048 | 29.791 | 54.852 | 1.00 | 24.35 |
| 391 | CA | GLU | 186 | 17.239 | 31.159 | 54.382 | 1.00 | 29.90 |
| 392 | CB | GLU | 186 | 17.324 | 31.191 | 52.856 | 1.00 | 34.25 |
| 393 | CG | GLU | 186 | 18.410 | 30.325 | 52.267 | 1.00 | 44.55 |
| 394 | CD | GLU | 186 | 18.041 | 29.815 | 50.890 | 1.00 | 52.24 |
| 395 | OE1 | GLU | 186 | 17.076 | 29.019 | 50.785 | 1.00 | 55.72 |
| 396 | OE2 | GLU | 186 | 18.712 | 30.216 | 49.912 | 1.00 | 56.27 |
| 397 | C | GLU | 186 | 16.058 | 32.019 | 54.835 | 1.00 | 30.26 |
| 398 | O | GLU | 186 | 15.028 | 31.493 | 55.258 | 1.00 | 28.54 |
| 399 | N | PRO | 187 | 16.192 | 33.356 | 54.750 | 1.00 | 30.75 |
| 400 | CD | PRO | 187 | 17.372 | 34.093 | 54.260 | 1.00 | 31.36 |
| 401 | CA | PRO | 187 | 15.110 | 34.265 | 55.158 | 1.00 | 29.11 |
| 402 | CB | PRO | 187 | 15.816 | 35.602 | 55.328 | 1.00 | 30.58 |
| 403 | CG | PRO | 187 | 16.940 | 35.543 | 54.338 | 1.00 | 32.46 |
| 404 | C | PRO | 187 | 14.039 | 34.321 | 54.077 | 1.00 | 26.13 |
| 405 | O | PRO | 187 | 13.716 | 35.389 | 53.554 | 1.00 | 26.30 |
| 406 | N | VAL | 188 | 13.491 | 33.156 | 53.755 | 1.00 | 25.66 |
| 407 | CA | VAL | 188 | 12.475 | 33.039 | 52.720 | 1.00 | 23.68 |
| 408 | CB | VAL | 188 | 12.834 | 31.887 | 51.753 | 1.00 | 25.26 |
| 409 | CG1 | VAL | 188 | 14.007 | 32.304 | 50.873 | 1.00 | 25.39 |
| 410 | CG2 | VAL | 188 | 13.190 | 30.622 | 52.544 | 1.00 | 22.93 |
| 411 | C | VAL | 188 | 11.074 | 32.801 | 53.283 | 1.00 | 23.95 |
| 412 | O | VAL | 188 | 10.169 | 32.384 | 52.555 | 1.00 | 22.87 |
| 413 | N | GLY | 189 | 10.901 | 33.076 | 54.575 | 1.00 | 23.30 |
| 414 | CA | GLY | 189 | 9.614 | 32.874 | 55.216 | 1.00 | 21.73 |
| 415 | C | GLY | 189 | 8.770 | 34.130 | 55.325 | 1.00 | 23.38 |
| 416 | O | GLY | 189 | 9.090 | 35.159 | 54.726 | 1.00 | 22.03 |
| 417 | N | LYS | 190 | 7.691 | 34.042 | 56.101 | 1.00 | 22.49 |
| 418 | CA | LYS | 190 | 6.778 | 35.163 | 56.294 | 1.00 | 22.94 |
| 419 | CB | LYS | 190 | 5.365 | 34.783 | 55.834 | 1.00 | 21.84 |
| 420 | CG | LYS | 190 | 5.326 | 34.038 | 54.525 | 1.00 | 26.57 |
| 421 | CD | LYS | 190 | 4.321 | 34.648 | 53.572 | 1.00 | 30.81 |
| 422 | CE | LYS | 190 | 4.357 | 33.943 | 52.222 | 1.00 | 36.07 |
| 423 | NZ | LYS | 190 | 4.792 | 34.858 | 51.130 | 1.00 | 39.80 |
| 424 | C | LYS | 190 | 6.702 | 35.654 | 57.739 | 1.00 | 23.72 |
| 425 | O | LYS | 190 | 6.061 | 36.671 | 58.015 | 1.00 | 25.75 |
| 426 | N | ASN | 191 | 7.344 | 34.948 | 58.663 | 1.00 | 21.28 |
| 427 | CA | ASN | 191 | 7.277 | 35.363 | 60.058 | 1.00 | 21.94 |
| 428 | CB | ASN | 191 | 6.044 | 34.735 | 60.722 | 1.00 | 20.60 |
| 429 | CG | ASN | 191 | 6.087 | 33.219 | 60.731 | 1.00 | 18.35 |
| 430 | OD1 | ASN | 191 | 5.189 | 32.555 | 60.212 | 1.00 | 23.94 |
| 431 | ND2 | ASN | 191 | 7.124 | 32.665 | 61.322 | 1.00 | 16.21 |
| 432 | C | ASN | 191 | 8.519 | 35.053 | 60.885 | 1.00 | 22.37 |
| 433 | O | ASN | 191 | 9.497 | 34.508 | 60.384 | 1.00 | 21.11 |
| 434 | N | ASP | 192 | 8.460 | 35.413 | 62.164 | 1.00 | 23.15 |
| 435 | CA | ASP | 192 | 9.558 | 35.183 | 63.095 | 1.00 | 22.51 |
| 436 | CB | ASP | 192 | 9.903 | 36.484 | 63.815 | 1.00 | 23.70 |
| 437 | CG | ASP | 192 | 8.808 | 36.922 | 64.774 | 1.00 | 29.04 |
| 438 | OD1 | ASP | 192 | 7.615 | 36.708 | 64.464 | 1.00 | 28.33 |
| 439 | OD2 | ASP | 192 | 9.138 | 37.478 | 65.844 | 1.00 | 30.02 |
| 440 | C | ASP | 192 | 9.145 | 34.129 | 64.123 | 1.00 | 22.10 |
| 441 | O | ASP | 192 | 9.680 | 34.089 | 65.233 | 1.00 | 22.44 |
| 442 | N | GLY | 193 | 8.190 | 33.280 | 63.748 | 1.00 | 20.52 |
| 443 | CA | GLY | 193 | 7.708 | 32.245 | 64.651 | 1.00 | 18.33 |
| 444 | C | GLY | 193 | 6.364 | 32.590 | 65.274 | 1.00 | 19.16 |
| 445 | O | GLY | 193 | 5.745 | 31.765 | 65.942 | 1.00 | 19.96 |
| 446 | N | SER | 194 | 5.904 | 33.817 | 65.052 | 1.00 | 20.43 |
| 447 | CA | SER | 194 | 4.630 | 34.267 | 65.597 | 1.00 | 20.23 |
| 448 | CB | SER | 194 | 4.872 | 35.349 | 66.656 | 1.00 | 19.09 |
| 449 | CG | SER | 194 | 5.140 | 36.599 | 66.047 | 1.00 | 19.84 |
| 450 | C | SER | 194 | 3.699 | 34.814 | 64.512 | 1.00 | 20.33 |
| 451 | O | SER | 194 | 4.149 | 35.388 | 63.514 | 1.00 | 20.77 |
| 452 | N | VAL | 195 | 2.398 | 34.615 | 64.711 | 1.00 | 18.51 |
| 453 | CA | VAL | 195 | 1.392 | 35.105 | 63.779 | 1.00 | 16.29 |
| 454 | CB | VAL | 195 | 0.721 | 33.947 | 62.995 | 1.00 | 18.73 |
| 455 | CG1 | VAL | 195 | −0.425 | 34.483 | 62.146 | 1.00 | 17.46 |
| 456 | CG2 | VAL | 195 | 1.748 | 33.256 | 62.101 | 1.00 | 16.39 |
| 457 | C | VAL | 195 | 0.342 | 35.826 | 64.616 | 1.00 | 14.96 |
| 458 | O | VAL | 195 | −0.042 | 35.347 | 65.677 | 1.00 | 13.86 |
| 459 | N | ALA | 196 | −0.097 | 36.987 | 64.146 | 1.00 | 15.82 |
| 460 | CA | ALA | 196 | −1.106 | 37.776 | 64.846 | 1.00 | 17.66 |
| 461 | CB | ALA | 196 | −2.472 | 37.102 | 64.733 | 1.00 | 12.63 |
| 462 | C | ALA | 196 | −0.763 | 38.008 | 66.314 | 1.00 | 18.77 |
| 463 | O | ALA | 196 | −1.651 | 38.043 | 67.164 | 1.00 | 19.10 |
| 464 | N | GLY | 197 | 0.522 | 38.154 | 66.617 | 1.00 | 19.76 |
| 465 | CA | GLY | 197 | 0.924 | 38.402 | 67.990 | 1.00 | 19.28 |
| 466 | C | GLY | 197 | 1.054 | 37.185 | 68.886 | 1.00 | 20.94 |
| 467 | O | GLY | 197 | 1.414 | 37.313 | 70.053 | 1.00 | 24.66 |
| 468 | N | VAL | 198 | 0.752 | 36.007 | 68.353 | 1.00 | 20.39 |
| 469 | CA | VAL | 198 | 0.859 | 34.766 | 69.113 | 1.00 | 19.76 |
| 470 | CB | VAL | 198 | −0.334 | 33.837 | 68.823 | 1.00 | 19.45 |
| 471 | CG1 | VAL | 198 | −0.251 | 32.596 | 69.693 | 1.00 | 17.99 |
| 472 | CG2 | VAL | 198 | −1.635 | 34.580 | 69.052 | 1.00 | 19.78 |
| 473 | C | VAL | 198 | 2.138 | 34.055 | 68.679 | 1.00 | 20.14 |
| 474 | O | VAL | 198 | 2.325 | 33.786 | 67.490 | 1.00 | 20.40 |
| 475 | N | ARG | 199 | 3.019 | 33.760 | 69.632 | 1.00 | 21.11 |
| 476 | CA | ARG | 199 | 4.270 | 33.074 | 69.312 | 1.00 | 22.59 |
| 477 | CB | ARG | 199 | 5.372 | 33.430 | 70.315 | 1.00 | 24.86 |
| 478 | CG | ARG | 199 | 6.663 | 32.639 | 70.095 | 1.00 | 24.38 |
| 479 | CD | ARG | 199 | 7.904 | 33.438 | 70.474 | 1.00 | 26.43 |
| 480 | NE | ARG | 199 | 7.902 | 34.787 | 69.912 | 1.00 | 27.34 |
| 481 | CZ | ARG | 199 | 8.319 | 35.096 | 68.688 | 1.00 | 27.49 |
| 482 | NH1 | ARG | 199 | 8.780 | 34.157 | 67.873 | 1.00 | 25.26 |
| 483 | NH2 | ARG | 199 | 8.275 | 36.353 | 68.273 | 1.00 | 27.96 |
| 484 | C | ARG | 199 | 4.060 | 31.573 | 69.333 | 1.00 | 22.36 |
| 485 | O | ARG | 199 | 3.556 | 31.029 | 70.313 | 1.00 | 25.36 |
| 486 | N | TYR | 200 | 4.447 | 30.900 | 68.255 | 1.00 | 21.21 |
| 487 | CA | TYR | 200 | 4.297 | 29.452 | 68.180 | 1.00 | 20.20 |
| 488 | CB | TYR | 200 | 3.624 | 29.061 | 66.861 | 1.00 | 17.43 |
| 489 | CG | TYR | 200 | 2.187 | 29.543 | 66.787 | 1.00 | 20.26 |
| 490 | CD1 | TYR | 200 | 1.866 | 30.766 | 66.186 | 1.00 | 19.67 |
| 491 | CE1 | TYR | 200 | 0.547 | 31.232 | 66.152 | 1.00 | 17.26 |
| 492 | CD2 | TYR | 200 | 1.149 | 28.795 | 67.351 | 1.00 | 19.77 |
| 493 | CE2 | TYR | 200 | −0.173 | 29.254 | 67.322 | 1.00 | 17.74 |
| 494 | CZ | TYR | 200 | −0.462 | 30.471 | 66.722 | 1.00 | 17.74 |
| 495 | OH | TYR | 200 | −1.760 | 30.924 | 66.696 | 1.00 | 18.38 |
| 496 | C | TYR | 200 | 5.662 | 28.801 | 68.325 | 1.00 | 20.58 |
| 497 | O | TYR | 200 | 5.795 | 27.750 | 68.952 | 1.00 | 25.13 |
| 498 | N | PHE | 201 | 6.674 | 29.433 | 67.745 | 1.00 | 21.61 |
| 499 | CA | PHE | 201 | 8.043 | 28.945 | 67.843 | 1.00 | 22.11 |
| 500 | CB | PHE | 201 | 8.349 | 27.871 | 66.782 | 1.00 | 20.39 |
| 501 | CG | PHE | 201 | 8.171 | 28.326 | 65.363 | 1.00 | 20.23 |
| 502 | CD1 | PHE | 201 | 9.254 | 28.797 | 64.629 | 1.00 | 21.85 |
| 503 | CD2 | PHE | 201 | 6.935 | 28.222 | 64.735 | 1.00 | 20.75 |
| 504 | CE1 | PHE | 201 | 9.114 | 29.156 | 63.282 | 1.00 | 18.71 |
| 505 | CE2 | PHE | 201 | 6.785 | 28.579 | 63.390 | 1.00 | 21.52 |
| 506 | CZ | PHE | 201 | 7.882 | 29.046 | 62.664 | 1.00 | 21.08 |
| 507 | C | PHE | 201 | 8.997 | 30.121 | 67.719 | 1.00 | 24.26 |
| 508 | O | PHE | 201 | 8.567 | 31.257 | 67.517 | 1.00 | 21.62 |
| 509 | N | ASP | 202 | 10.290 | 29.851 | 67.849 | 1.00 | 26.83 |
| 510 | CA | ASP | 202 | 11.292 | 30.906 | 67.789 | 1.00 | 28.71 |
| 511 | CB | ASP | 202 | 12.149 | 30.883 | 69.054 | 1.00 | 33.70 |
| 512 | CG | ASP | 202 | 11.401 | 31.365 | 70.267 | 1.00 | 39.70 |
| 513 | OD1 | ASP | 202 | 11.080 | 32.575 | 70.315 | 1.00 | 44.55 |
| 514 | OD2 | ASP | 202 | 11.138 | 30.536 | 71.167 | 1.00 | 42.64 |
| 515 | C | ASP | 202 | 12.220 | 30.843 | 66.596 | 1.00 | 26.97 |
| 516 | O | ASP | 202 | 12.685 | 29.771 | 66.213 | 1.00 | 25.73 |
| 517 | N | GYS | 203 | 12.494 | 32.015 | 66.034 | 1.00 | 25.65 |
| 518 | CA | GYS | 203 | 13.400 | 32.157 | 64.904 | 1.00 | 27.80 |
| 519 | CB | GYS | 203 | 12.953 | 31.274 | 63.721 | 1.00 | 28.41 |
| 520 | SG | GYS | 203 | 11.589 | 31.891 | 62.715 | 1.00 | 28.23 |
| 521 | C | GYS | 203 | 13.490 | 33.627 | 64.499 | 1.00 | 28.61 |
| 522 | O | GYS | 203 | 12.683 | 34.456 | 64.927 | 1.00 | 27.75 |
| 523 | N | ASP | 204 | 14.492 | 33.951 | 63.694 | 1.00 | 29.32 |
| 524 | CA | ASP | 204 | 14.682 | 35.322 | 63.253 | 1.00 | 31.69 |
| 525 | CB | ASP | 204 | 16.061 | 35.477 | 62.590 | 1.00 | 35.33 |
| 526 | CG | ASP | 204 | 17.222 | 35.165 | 63.542 | 1.00 | 41.00 |
| 527 | OD1 | ASP | 204 | 18.248 | 34.623 | 63.066 | 1.00 | 45.86 |
| 528 | OD2 | ASP | 204 | 17.116 | 35.460 | 64.757 | 1.00 | 38.06 |
| 529 | C | ASP | 204 | 13.588 | 35.706 | 62.263 | 1.00 | 31.25 |
| 530 | O | ASP | 204 | 12.984 | 34.841 | 61.628 | 1.00 | 32.37 |
| 531 | N | PRO | 205 | 13.295 | 37.012 | 62.142 | 1.00 | 30.83 |
| 532 | CD | PRO | 205 | 13.886 | 38.118 | 62.913 | 1.00 | 31.37 |
| 533 | CA | PRO | 205 | 12.263 | 37.483 | 61.207 | 1.00 | 29.25 |
| 534 | CB | PRO | 205 | 12.357 | 39.006 | 61.296 | 1.00 | 29.12 |
| 535 | CG | PRO | 205 | 12.967 | 39.271 | 62.620 | 1.00 | 30.55 |
| 536 | C | PRO | 205 | 12.511 | 36.989 | 59.785 | 1.00 | 28.31 |
| 537 | O | PRO | 205 | 13.656 | 36.912 | 59.328 | 1.00 | 26.02 |
| 538 | N | LYS | 206 | 11.424 | 36.653 | 59.098 | 1.00 | 28.10 |
| 539 | CA | LYS | 206 | 11.475 | 36.168 | 57.726 | 1.00 | 28.09 |
| 540 | CB | LYS | 206 | 12.301 | 37.126 | 56.859 | 1.00 | 33.43 |
| 541 | CG | LYS | 206 | 11.477 | 37.991 | 55.921 | 1.00 | 41.18 |
| 542 | CD | LYS | 206 | 10.774 | 39.106 | 56.681 | 1.00 | 46.96 |
| 543 | CE | LYS | 206 | 9.279 | 38.845 | 56.811 | 1.00 | 52.64 |
| 544 | NZ | LYS | 206 | 8.700 | 39.528 | 58.009 | 1.00 | 54.49 |
| 545 | C | LYS | 206 | 12.021 | 34.743 | 57.584 | 1.00 | 25.55 |
| 546 | O | LYS | 206 | 12.163 | 34.250 | 56.467 | 1.00 | 25.80 |
| 547 | N | TYR | 207 | 12.324 | 34.085 | 58.703 | 1.00 | 22.35 |
| 548 | CA | TYR | 207 | 12.843 | 32.710 | 58.669 | 1.00 | 22.72 |
| 549 | CB | TYR | 207 | 14.032 | 32.556 | 59.624 | 1.00 | 24.77 |
| 550 | CG | TYR | 207 | 15.338 | 33.056 | 59.062 | 1.00 | 27.31 |
| 551 | CD1 | TYR | 207 | 15.735 | 34.381 | 59.254 | 1.00 | 28.20 |
| 552 | CE1 | TYR | 207 | 16.938 | 34.850 | 58.737 | 1.00 | 29.92 |
| 553 | CD2 | TYR | 207 | 16.179 | 32.209 | 58.336 | 1.00 | 28.04 |
| 554 | CE2 | TYR | 207 | 17.390 | 32.668 | 57.811 | 1.00 | 27.39 |
| 555 | CZ | TYR | 207 | 17.759 | 33.989 | 58.017 | 1.00 | 29.69 |
| 556 | OH | TYR | 207 | 18.946 | 34.458 | 57.511 | 1.00 | 30.44 |
| 557 | C | TYR | 207 | 11.793 | 31.652 | 59.035 | 1.00 | 21.40 |
| 558 | O | TYR | 207 | 12.063 | 30.452 | 58.980 | 1.00 | 21.28 |
| 559 | N | GLY | 208 | 10.601 | 32.092 | 59.417 | 1.00 | 18.03 |
| 560 | CA | GLY | 208 | 9.574 | 31.139 | 59.780 | 1.00 | 15.50 |
| 561 | C | GLY | 208 | 8.412 | 31.148 | 58.816 | 1.00 | 16.29 |
| 562 | O | GLY | 208 | 8.151 | 32.145 | 58.145 | 1.00 | 17.50 |
| 563 | N | GLY | 209 | 7.707 | 30.029 | 58.747 | 1.00 | 16.55 |
| 564 | CA | GLY | 209 | 6.561 | 29.946 | 57.867 | 1.00 | 15.32 |
| 565 | C | GLY | 209 | 5.594 | 28.881 | 58.331 | 1.00 | 15.92 |
| 566 | O | GLY | 209 | 6.003 | 27.876 | 58.905 | 1.00 | 18.17 |
| 567 | N | PHE | 210 | 4.308 | 29.113 | 58.105 | 1.00 | 14.90 |
| 568 | CA | PHE | 210 | 3.282 | 28.146 | 58.463 | 1.00 | 16.74 |
| 569 | CB | PHE | 210 | 2.222 | 28.774 | 59.374 | 1.00 | 16.17 |
| 570 | CG | PHE | 210 | 2.519 | 28.634 | 60.842 | 1.00 | 17.20 |
| 571 | CD1 | PHE | 210 | 2.875 | 29.748 | 61.601 | 1.00 | 18.61 |
| 572 | CD2 | PHE | 210 | 2.433 | 27.394 | 61.469 | 1.00 | 17.88 |
| 573 | GE1 | PHE | 210 | 3.143 | 29.630 | 62.974 | 1.00 | 21.83 |
| 574 | GE2 | PHE | 210 | 2.698 | 27.261 | 62.834 | 1.00 | 21.16 |
| 575 | CZ | PHE | 210 | 3.054 | 28.385 | 63.588 | 1.00 | 20.25 |
| 576 | C | PHE | 210 | 2.630 | 27.682 | 57.161 | 1.00 | 17.37 |
| 577 | O | PHE | 210 | 2.191 | 28.503 | 56.349 | 1.00 | 16.88 |
| 578 | N | VAL | 211 | 2.585 | 26.368 | 56.961 | 1.00 | 15.90 |
| 579 | CA | VAL | 211 | 1.988 | 25.792 | 55.761 | 1.00 | 15.45 |
| 580 | CB | VAL | 211 | 3.072 | 25.408 | 54.709 | 1.00 | 15.30 |
| 581 | CG1 | VAL | 211 | 3.885 | 26.631 | 54.307 | 1.00 | 12.68 |
| 582 | CG2 | VAL | 211 | 3.989 | 24.322 | 55.276 | 1.00 | 13.86 |
| 583 | C | VAL | 211 | 1.212 | 24.532 | 56.125 | 1.00 | 15.36 |
| 584 | O | VAL | 211 | 1.372 | 23.994 | 57.218 | 1.00 | 16.47 |
| 585 | N | ARG | 212 | 0.378 | 24.063 | 55.206 | 1.00 | 17.40 |
| 586 | CA | ARG | 212 | −0.393 | 22.851 | 55.439 | 1.00 | 18.81 |
| 587 | CB | ARG | 212 | −1.542 | 22.752 | 54.437 | 1.00 | 17.70 |
| 588 | CG | ARG | 212 | −2.629 | 23.786 | 54.661 | 1.00 | 20.41 |
| 589 | CD | ARG | 212 | −3.358 | 23.573 | 55.992 | 1.00 | 19.64 |
| 590 | NE | ARG | 212 | −4.607 | 24.333 | 56.047 | 1.00 | 21.34 |
| 591 | CZ | ARG | 212 | −5.364 | 24.466 | 57.131 | 1.00 | 17.56 |
| 592 | NH1 | ARG | 212 | −5.005 | 23.886 | 58.267 | 1.00 | 18.10 |
| 593 | NH2 | ARG | 212 | −6.468 | 25.197 | 57.083 | 1.00 | 16.84 |
| 594 | C | ARG | 212 | 0.522 | 21.635 | 55.300 | 1.00 | 20.03 |
| 595 | O | ARG | 212 | 1.491 | 21.659 | 54.534 | 1.00 | 18.23 |
| 596 | N | PRO | 213 | 0.233 | 20.558 | 56.050 | 1.00 | 21.79 |
| 597 | CD | PRO | 213 | −0.861 | 20.417 | 57.030 | 1.00 | 22.12 |
| 598 | CA | PRO | 213 | 1.062 | 19.349 | 55.978 | 1.00 | 22.62 |
| 599 | CB | PRO | 213 | 0.322 | 18.352 | 56.872 | 1.00 | 24.14 |
| 600 | CG | PRO | 213 | −0.441 | 19.214 | 57.838 | 1.00 | 20.67 |
| 601 | C | PRO | 213 | 1.296 | 18.810 | 54.563 | 1.00 | 23.37 |
| 602 | O | PRO | 213 | 2.385 | 18.326 | 54.263 | 1.00 | 24.67 |
| 603 | N | VAL | 214 | 0.298 | 18.911 | 53.688 | 1.00 | 23.11 |
| 604 | CA | VAL | 214 | 0.451 | 18.409 | 52.323 | 1.00 | 24.19 |
| 605 | CB | VAL | 214 | −0.873 | 18.501 | 51.518 | 1.00 | 24.82 |
| 606 | CG1 | VAL | 214 | −1.900 | 17.543 | 52.100 | 1.00 | 26.57 |
| 607 | CG2 | VAL | 214 | −1.388 | 19.934 | 51.504 | 1.00 | 24.69 |
| 608 | C | VAL | 214 | 1.544 | 19.107 | 51.510 | 1.00 | 23.73 |
| 609 | O | VAL | 214 | 1.966 | 18.607 | 50.464 | 1.00 | 24.84 |
| 610 | N | ASP | 215 | 2.004 | 20.259 | 51.986 | 1.00 | 22.33 |
| 611 | CA | ASP | 215 | 3.037 | 21.011 | 51.283 | 1.00 | 22.26 |
| 612 | CB | ASP | 215 | 2.707 | 22.501 | 51.338 | 1.00 | 21.96 |
| 613 | CG | ASP | 215 | 1.436 | 22.832 | 50.598 | 1.00 | 28.29 |
| 614 | OD1 | ASP | 215 | 1.106 | 22.095 | 49.646 | 1.00 | 30.46 |
| 615 | OD2 | ASP | 215 | 0.763 | 23.822 | 50.959 | 1.00 | 32.65 |
| 616 | C | ASP | 215 | 4.417 | 20.765 | 51.874 | 1.00 | 20.14 |
| 617 | O | ASP | 215 | 5.400 | 21.383 | 51.471 | 1.00 | 20.74 |
| 618 | N | VAL | 216 | 4.477 | 19.843 | 52.824 | 1.00 | 22.36 |
| 619 | CA | VAL | 216 | 5.719 | 19.525 | 53.510 | 1.00 | 24.04 |
| 620 | CB | VAL | 216 | 5.601 | 19.790 | 55.030 | 1.00 | 25.20 |
| 621 | CG1 | VAL | 216 | 6.956 | 19.615 | 55.694 | 1.00 | 23.32 |
| 622 | CG2 | VAL | 216 | 5.055 | 21.187 | 55.277 | 1.00 | 22.12 |
| 623 | C | VAL | 216 | 6.156 | 18.079 | 53.340 | 1.00 | 26.07 |
| 624 | O | VAL | 216 | 5.365 | 17.149 | 53.518 | 1.00 | 26.51 |
| 625 | N | LYS | 217 | 7.426 | 17.911 | 52.989 | 1.00 | 24.78 |
| 626 | CA | LYS | 217 | 8.031 | 16.602 | 52.826 | 1.00 | 25.38 |
| 627 | CB | LYS | 217 | 8.706 | 16.484 | 51.461 | 1.00 | 27.84 |
| 628 | CG | LYS | 217 | 7.848 | 15.819 | 50.408 | 1.00 | 34.88 |
| 629 | CD | LYS | 217 | 8.314 | 16.191 | 49.010 | 1.00 | 42.96 |
| 630 | CE | LYS | 217 | 9.371 | 15.217 | 48.500 | 1.00 | 47.27 |
| 631 | NZ | LYS | 217 | 10.273 | 15.820 | 47.469 | 1.00 | 49.15 |
| 632 | C | LYS | 217 | 9.072 | 16.555 | 53.933 | 1.00 | 24.48 |
| 633 | O | LYS | 217 | 9.900 | 17.456 | 54.058 | 1.00 | 24.51 |
| 634 | N | VAL | 218 | 9.023 | 15.517 | 54.752 | 1.00 | 23.97 |
| 635 | CA | VAL | 218 | 9.962 | 15.401 | 55.853 | 1.00 | 25.68 |
| 636 | CB | VAL | 218 | 9.186 | 15.093 | 57.161 | 1.00 | 26.82 |
| 637 | CG1 | VAL | 218 | 9.599 | 13.758 | 57.741 | 1.00 | 28.68 |
| 638 | CG2 | VAL | 218 | 9.394 | 16.220 | 58.149 | 1.00 | 29.06 |
| 639 | C | VAL | 218 | 11.010 | 14.331 | 55.564 | 1.00 | 25.91 |
| 640 | O | VAL | 218 | 10.735 | 13.354 | 54.876 | 1.00 | 24.68 |
| 641 | N | GLY | 219 | 12.222 | 14.526 | 56.066 | 1.00 | 25.54 |
| 642 | CA | GLY | 219 | 13.249 | 13.533 | 55.832 | 1.00 | 26.45 |
| 643 | C | GLY | 219 | 14.649 | 14.093 | 55.813 | 1.00 | 26.33 |
| 644 | O | GLY | 219 | 14.929 | 15.116 | 56.433 | 1.00 | 28.35 |
| 645 | N | ASP | 220 | 15.537 | 13.407 | 55.105 | 1.00 | 26.24 |
| 646 | CA | ASP | 220 | 16.917 | 13.843 | 54.995 | 1.00 | 25.72 |
| 647 | CB | ASP | 220 | 17.817 | 12.658 | 54.648 | 1.00 | 29.29 |
| 648 | CG | ASP | 220 | 19.290 | 13.003 | 54.725 | 1.00 | 33.58 |
| 649 | OD1 | ASP | 220 | 19.616 | 14.127 | 55.163 | 1.00 | 36.10 |
| 650 | OD2 | ASP | 220 | 20.124 | 12.151 | 54.344 | 1.00 | 37.97 |
| 651 | C | ASP | 220 | 17.015 | 14.917 | 53.914 | 1.00 | 26.39 |
| 652 | O | ASP | 220 | 17.237 | 14.627 | 52.733 | 1.00 | 27.16 |
| 653 | N | PHE | 221 | 16.820 | 16.163 | 54.329 | 1.00 | 23.69 |
| 654 | CA | PHE | 221 | 16.892 | 17.294 | 53.418 | 1.00 | 22.47 |
| 655 | CB | PHE | 221 | 15.522 | 17.964 | 53.299 | 1.00 | 21.04 |
| 656 | CG | PHE | 221 | 14.497 | 17.131 | 52.565 | 1.00 | 22.73 |
| 657 | OD1 | PHE | 221 | 13.513 | 16.428 | 53.269 | 1.00 | 21.74 |
| 658 | OD2 | PHE | 221 | 14.485 | 17.084 | 51.169 | 1.00 | 20.35 |
| 659 | CE1 | PHE | 221 | 12.532 | 15.699 | 52.599 | 1.00 | 17.10 |
| 660 | CE2 | PHE | 221 | 13.505 | 16.356 | 50.490 | 1.00 | 21.18 |
| 661 | CZ | PHE | 221 | 12.525 | 15.662 | 51.210 | 1.00 | 16.58 |
| 662 | C | PHE | 221 | 17.914 | 18.261 | 54.004 | 1.00 | 23.41 |
| 663 | O | PHE | 221 | 17.562 | 19.192 | 54.734 | 1.00 | 23.82 |
| 664 | N | PRO | 222 | 19.203 | 18.047 | 53.683 | 1.00 | 23.08 |
| 665 | CD | PRO | 222 | 19.709 | 17.004 | 52.770 | 1.00 | 24.09 |
| 666 | CA | PRO | 222 | 20.284 | 18.894 | 54.182 | 1.00 | 20.93 |
| 667 | CB | PRO | 222 | 21.538 | 18.133 | 53.788 | 1.00 | 22.27 |
| 668 | CG | PRO | 222 | 21.138 | 17.401 | 52.554 | 1.00 | 21.54 |
| 669 | C | PRO | 222 | 20.227 | 20.266 | 53.544 | 1.00 | 20.95 |
| 670 | O | PRO | 222 | 19.671 | 20.439 | 52.458 | 1.00 | 23.36 |
| 671 | N | GLU | 223 | 20.803 | 21.247 | 54.222 | 1.00 | 22.03 |
| 672 | CA | GLU | 223 | 20.803 | 22.604 | 53.712 | 1.00 | 20.74 |
| 673 | CB | GLU | 223 | 21.484 | 23.516 | 54.722 | 1.00 | 24.29 |
| 674 | CG | GLU | 223 | 21.788 | 24.885 | 54.191 | 1.00 | 29.58 |
| 675 | CD | GLU | 223 | 22.330 | 25.782 | 55.267 | 1.00 | 37.43 |
| 676 | OE1 | GLU | 223 | 22.895 | 25.247 | 56.248 | 1.00 | 41.67 |
| 677 | OE2 | GLU | 223 | 22.187 | 27.017 | 55.136 | 1.00 | 41.44 |
| 678 | C | GLU | 223 | 21.499 | 22.715 | 52.357 | 1.00 | 19.59 |
| 679 | O | GLU | 223 | 22.610 | 22.219 | 52.171 | 1.00 | 21.80 |
| 680 | N | LEU | 224 | 20.846 | 23.370 | 51.407 | 1.00 | 19.06 |
| 681 | CA | LEU | 224 | 21.424 | 23.536 | 50.084 | 1.00 | 18.88 |
| 682 | CB | LEU | 224 | 20.322 | 23.821 | 49.062 | 1.00 | 18.64 |
| 683 | CG | LEU | 224 | 19.465 | 22.583 | 48.769 | 1.00 | 22.10 |
| 684 | OD1 | LEU | 224 | 18.124 | 23.007 | 48.186 | 1.00 | 21.33 |
| 685 | OD2 | LEU | 224 | 20.210 | 21.645 | 47.811 | 1.00 | 21.76 |
| 686 | C | LEU | 224 | 22.468 | 24.651 | 50.069 | 1.00 | 21.12 |
| 687 | O | LEU | 224 | 22.156 | 25.827 | 49.865 | 1.00 | 22.85 |
| 688 | N | SER | 225 | 23.718 | 24.267 | 50.285 | 1.00 | 21.17 |
| 689 | CA | SER | 225 | 24.810 | 25.225 | 50.306 | 1.00 | 22.01 |
| 690 | CB | SER | 225 | 24.978 | 25.799 | 51.716 | 1.00 | 22.53 |
| 691 | CG | SER | 225 | 25.848 | 26.917 | 51.707 | 1.00 | 29.80 |
| 692 | C | SER | 225 | 26.102 | 24.551 | 49.881 | 1.00 | 19.27 |
| 693 | O | SER | 225 | 26.308 | 23.367 | 50.142 | 1.00 | 19.36 |
| 694 | N | ILE | 226 | 26.963 | 25.317 | 49.223 | 1.00 | 19.24 |
| 695 | CA | ILE | 226 | 28.263 | 24.826 | 48.775 | 1.00 | 21.99 |
| 696 | CB | ILE | 226 | 28.346 | 24.762 | 47.240 | 1.00 | 20.05 |
| 697 | CG2 | ILE | 226 | 29.793 | 24.582 | 46.802 | 1.00 | 21.53 |
| 698 | CG1 | ILE | 226 | 27.477 | 23.615 | 46.725 | 1.00 | 19.70 |
| 699 | CD1 | ILE | 226 | 27.266 | 23.641 | 45.232 | 1.00 | 19.11 |
| 700 | C | ILE | 226 | 29.321 | 25.805 | 49.273 | 1.00 | 23.13 |
| 701 | O | ILE | 226 | 29.337 | 26.967 | 48.862 | 1.00 | 24.16 |
| 702 | N | ASP | 227 | 30.194 | 25.348 | 50.165 | 1.00 | 22.64 |
| 703 | CA | ASP | 227 | 31.239 | 26.223 | 50.688 | 1.00 | 24.83 |
| 704 | CB | ASP | 227 | 31.808 | 25.672 | 51.998 | 1.00 | 26.46 |
| 705 | CG | ASP | 227 | 30.828 | 25.784 | 53.156 | 1.00 | 31.97 |
| 706 | OD1 | ASP | 227 | 30.911 | 24.947 | 54.075 | 1.00 | 35.13 |
| 707 | OD2 | ASP | 227 | 29.978 | 26.703 | 53.153 | 1.00 | 33.41 |
| 708 | C | ASP | 227 | 32.349 | 26.345 | 49.658 | 1.00 | 23.02 |
| 709 | O | ASP | 227 | 32.815 | 27.445 | 49.358 | 1.00 | 24.46 |
| 710 | N | GLU | 228 | 32.754 | 25.202 | 49.114 | 1.00 | 20.23 |
| 711 | CA | GLU | 228 | 33.805 | 25.135 | 48.110 | 1.00 | 18.61 |
| 712 | CB | GLU | 228 | 35.187 | 25.157 | 48.765 | 1.00 | 17.51 |
| 713 | CG | GLU | 228 | 36.321 | 25.128 | 47.761 | 1.00 | 22.14 |
| 714 | CD | GLU | 228 | 37.673 | 25.285 | 48.417 | 1.00 | 26.26 |
| 715 | CE1 | GLU | 228 | 38.577 | 25.857 | 47.779 | 1.00 | 29.45 |
| 716 | CE2 | GLU | 228 | 37.833 | 24.837 | 49.570 | 1.00 | 30.48 |
| 717 | C | GLU | 228 | 33.645 | 23.839 | 47.343 | 1.00 | 17.19 |
| 718 | O | GLU | 228 | 33.354 | 22.802 | 47.926 | 1.00 | 16.62 |
| 719 | N | ILE | 229 | 33.849 | 23.899 | 46.036 | 1.00 | 17.73 |
| 720 | CA | ILE | 229 | 33.710 | 22.720 | 45.204 | 1.00 | 18.21 |
| 721 | CB | ILE | 229 | 32.299 | 22.680 | 44.559 | 1.00 | 19.03 |
| 722 | CG2 | ILE | 229 | 32.141 | 23.838 | 43.589 | 1.00 | 19.34 |
| 723 | CG1 | ILE | 229 | 32.054 | 21.323 | 43.884 | 1.00 | 17.72 |
| 724 | CD1 | ILE | 229 | 30.594 | 21.059 | 43.571 | 1.00 | 16.86 |
| 725 | C | ILE | 229 | 34.780 | 22.747 | 44.129 | 1.00 | 20.29 |
| 726 | O | ILE | 229 | 35.419 | 23.806 | 43.973 | 1.00 | 22.95 |
| 727 | OT | ILE | 229 | 34.973 | 21.714 | 43.462 | 1.00 | 22.27 |
| 728 | OH2 | TIP3 | 501 | −1.946 | 13.345 | 52.275 | 1.00 | 41.27 |
| 729 | OH2 | TIP3 | 502 | 6.636 | 14.012 | 54.959 | 1.00 | 43.65 |
| 730 | OH2 | TIP3 | 503 | 7.613 | 18.516 | 58.128 | 1.00 | 65.21 |
| 731 | OH2 | TIP3 | 504 | 13.390 | 9.645 | 61.917 | 1.00 | 68.67 |
| 732 | OH2 | TIP3 | 505 | 14.686 | 14.604 | 59.591 | 1.00 | 34.15 |
| 733 | OH2 | TIP3 | 506 | 17.790 | 15.056 | 58.793 | 1.00 | 41.76 |
| 734 | OH2 | TIP3 | 507 | 12.680 | 22.367 | 65.681 | 1.00 | 31.26 |
| 735 | OH2 | TIP3 | 508 | 11.237 | 27.198 | 68.446 | 1.00 | 38.37 |
| 736 | OH2 | TIP3 | 509 | 4.306 | 21.356 | 67.858 | 1.00 | 25.39 |
| 737 | OH2 | TIP3 | 510 | 9.918 | 25.049 | 68.333 | 1.00 | 30.47 |
| 738 | OH2 | TIP3 | 511 | 4.826 | 15.480 | 71.206 | 1.00 | 49.12 |
| 739 | OH2 | TIP3 | 512 | 1.400 | 21.640 | 69.131 | 1.00 | 43.94 |
| 740 | OH2 | TIP3 | 513 | 5.552 | 19.628 | 72.409 | 1.00 | 32.32 |
| 741 | OH2 | TIP3 | 514 | 6.704 | 25.752 | 70.259 | 1.00 | 54.07 |
| 742 | OH2 | TIP3 | 515 | 2.210 | 34.403 | 72.778 | 1.00 | 29.33 |
| 743 | OH2 | TIP3 | 516 | 4.775 | 38.124 | 69.130 | 1.00 | 40.75 |
| 744 | OH2 | TIP3 | 517 | 7.792 | 36.993 | 72.357 | 1.00 | 36.18 |
| 745 | OH2 | TIP3 | 518 | 2.940 | 38.490 | 65.074 | 1.00 | 20.93 |
| 746 | OH2 | TIP3 | 519 | 1.303 | 38.526 | 62.240 | 1.00 | 32.37 |
| 747 | OH2 | TIP3 | 520 | 9.185 | 38.140 | 60.246 | 1.00 | 34.88 |
| 748 | OH2 | TIP3 | 521 | 2.569 | 36.302 | 58.725 | 1.00 | 39.77 |
| 749 | OH2 | TIP3 | 522 | 3.115 | 37.160 | 61.027 | 1.00 | 38.93 |
| 750 | OH2 | TIP3 | 523 | 5.722 | 37.636 | 62.561 | 1.00 | 31.89 |
| 751 | OH2 | TIP3 | 524 | 12.062 | 37.275 | 66.392 | 1.00 | 35.63 |
| 752 | OH2 | TIP3 | 525 | 12.019 | 35.106 | 68.024 | 1.00 | 56.76 |
| 753 | OH2 | TIP3 | 526 | 16.379 | 31.766 | 62.786 | 1.00 | 33.83 |
| 754 | OH2 | TIP3 | 527 | 28.173 | 36.619 | 60.809 | 1.00 | 58.43 |
| 755 | OH2 | TIP3 | 528 | 21.157 | 32.253 | 56.858 | 1.00 | 53.16 |
| 756 | OH2 | TIP3 | 529 | 21 .366 | 28.396 | 48.439 | 1.00 | 34.62 |
| 757 | OH2 | TIP3 | 530 | 20.056 | 27.068 | 51.634 | 1.00 | 24.89 |
| 758 | OH2 | TIP3 | 531 | 18.477 | 24.941 | 52.161 | 1.00 | 19.28 |
| 759 | OH2 | TIP3 | 532 | 16.348 | 25.721 | 50.884 | 1.00 | 24.52 |
| 760 | OH2 | 11P3 | 533 | 14.170 | 29.183 | 48.047 | 1.00 | 48.09 |
| 761 | OH2 | TIP3 | 534 | 15.421 | 27.703 | 53.156 | 1.00 | 21.15 |
| 762 | OH2 | TIP3 | 535 | 12.779 | 34.211 | 44.829 | 1.00 | 63.90 |
| 763 | OH2 | TIP3 | 536 | 15.009 | 23.783 | 46.741 | 1.00 | 43.20 |
| 764 | OH2 | TIP3 | 537 | 14.792 | 21.321 | 48.211 | 1.00 | 28.84 |
| 765 | OH2 | TIP3 | 538 | 8.132 | 19.458 | 49.572 | 1.00 | 31.66 |
| 766 | OH2 | TIP3 | 539 | 8.104 | 26.620 | 48.729 | 1.00 | 30.32 |
| 767 | OH2 | TIP3 | 540 | −0.087 | 25.571 | 52.786 | 1.00 | 17.50 |
| 768 | OH2 | TIP3 | 541 | −5.307 | 19.210 | 51.241 | 1.00 | 39.41 |
| 769 | OH2 | TIP3 | 542 | −4.475 | 21.623 | 52.129 | 1.00 | 27.25 |
| 770 | OH2 | TIP3 | 543 | −13.253 | 15.240 | 38.542 | 1.00 | 70.52 |
| 771 | OH2 | TIP3 | 544 | −5.052 | 25.767 | 53.701 | 1.00 | 23.11 |
| 772 | OH2 | TIP3 | 545 | −7.160 | 21.234 | 56.101 | 1.00 | 41.58 |
| 773 | OH2 | TIP3 | 546 | −5.995 | 20.319 | 58.282 | 1.00 | 31.90 |
| 774 | OH2 | TIP3 | 547 | 3.809 | 15.693 | 66.057 | 1.00 | 45.46 |
| 775 | OH2 | TIP3 | 548 | −8.146 | 21.907 | 63.951 | 1.00 | 31.88 |
| 776 | OH2 | TIP3 | 549 | −4.066 | 19.227 | 65.385 | 1.00 | 30.56 |
| 777 | OH2 | TIP3 | 550 | 1.459 | 17.628 | 68.303 | 1.00 | 66.39 |
| 778 | OH2 | TIP3 | 551 | −8.753 | 25.055 | 79.147 | 1.00 | 62.40 |
| 779 | OH2 | TIP3 | 552 | 6.278 | 16.984 | 67.284 | 1.00 | 37.32 |
| 780 | OH2 | TIP3 | 553 | 16.387 | 25.491 | 59.447 | 1.00 | 26.53 |
| 781 | OH2 | TIP3 | 554 | 21 .462 | 22.830 | 58.645 | 1.00 | 40.96 |
| 782 | OH2 | TIP3 | 555 | 24.194 | 19.500 | 56.563 | 1.00 | 34.62 |
| 783 | OH2 | TIP3 | 556 | 20.715 | 17.963 | 57.913 | 1.00 | 32.76 |
| 784 | OH2 | TIP3 | 557 | 16.671 | 12.752 | 51.030 | 1.00 | 26.88 |
| 785 | OH2 | TIP3 | 558 | 14.371 | 12.262 | 51.284 | 1.00 | 43.70 |
| 786 | OH2 | TIP3 | 559 | 27.775 | 21.312 | 49.589 | 1.00 | 27.28 |
| 787 | OH2 | TIP3 | 560 | 25.852 | 24.555 | 55.158 | 1.00 | 43.44 |
| 788 | OH2 | TIP3 | 561 | 33.801 | 24.886 | 54.945 | 1.00 | 60.01 |
| 789 | OH2 | TIP3 | 562 | 32.670 | 25.681 | 58.272 | 1.00 | 64.80 |
| 790 | OH2 | TIP3 | 563 | 36.187 | 25.446 | 52.667 | 1.00 | 53.63 |
| 791 | OH2 | TIP3 | 564 | 29.839 | 31.985 | 51.863 | 1.00 | 58.10 |
| 792 | OH2 | TIP3 | 565 | 29.226 | 28.913 | 51.889 | 1.00 | 40.53 |
| 793 | OH2 | 11P3 | 566 | 16.067 | 38.494 | 59.519 | 1.00 | 33.67 |
| 794 | OH2 | TIP3 | 567 | 7.690 | 43.583 | 55.837 | 1.00 | 47.10 |
| 795 | OH2 | TIP3 | 568 | −2.121 | 33.361 | 65.729 | 1.00 | 15.45 |
| 796 | OH2 | TIP3 | 569 | −4.393 | 37.297 | 67.868 | 1.00 | 20.59 |
| 797 | OH2 | TIP3 | 570 | −2.180 | 28.419 | 76.536 | 1.00 | 58.30 |
| 798 | OH2 | TIP3 | 571 | −7.115 | 23.775 | 53.633 | 1.00 | 37.81 |
| 799 | OH2 | TIP3 | 572 | −6.813 | 26.213 | 53.074 | 1.00 | 14.19 |
| 800 | OH2 | TIP3 | 573 | −8.670 | 31.651 | 55.017 | 1.00 | 18.02 |
| 801 | OH2 | TIP3 | 574 | 22.577 | 29.573 | 50.514 | 1.00 | 32.23 |
| 802 | OH2 | TIP3 | 575 | −10.362 | 31.326 | 56.980 | 1.00 | 38.74 |
| 803 | OH2 | TIP3 | 576 | 18.884 | 16.435 | 56.581 | 1.00 | 27.95 |
| 804 | OH2 | TIP3 | 577 | 21.637 | 28.199 | 52.868 | 1.00 | 32.16 |
| 805 | OH2 | TIP3 | 578 | 19.539 | 37.905 | 60.425 | 1.00 | 53.43 |
| 806 | OH2 | TIP3 | 579 | 8.224 | 23.791 | 70.104 | 1.00 | 48.90 |
| 807 | OH2 | TIP3 | 580 | −8.908 | 31.374 | 59.774 | 1.00 | 35.20 |
| 808 | OH2 | TIP3 | 581 | 18.125 | 22.910 | 60.880 | 1.00 | 33.75 |
| 809 | OH2 | TIP3 | 582 | 3.969 | 14.939 | 54.495 | 1.00 | 43.50 |
| 810 | OH2 | TIP3 | 583 | −12.195 | 16.867 | 34.385 | 1.00 | 53.69 |
| 811 | OH2 | TIP3 | 584 | 28.668 | 8.763 | 62.158 | 1.00 | 30.18 |
| 812 | OH2 | TIP3 | 585 | −6.227 | 27.836 | 53.858 | 1.00 | 21.81 |
| 813 | OH2 | TIP3 | 586 | 16.401 | 16.638 | 66.955 | 1.00 | 35.79 |
| 814 | OH2 | TIP3 | 587 | 3.154 | 20.575 | 47.621 | 1.00 | 33.65 |

REMARK r = 0.224693 free_{—L r = 0.294006}
REMARK DATE:13-Aug-01 10:25:38 created by user: songlin
CRYST1 64.156 64.156 101.946 90.00 90.00 120.00

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.015587 | 0.008999 | 0.000000 | 0.00000 |
| SCALE2 | 0.000000 | 0.017998 | 0.000000 | 0.00000 |
| SCALE3 | 0.000000 | 0.000000 | 0.009809 | 0.00000 |

What is claimed is:

1. A polypeptide comprising amino acid sequence of the CAP-Gly domain and heterologous amino acid sequence.

2. The polypeptide of claim 1, wherein the amino acid sequence of the CAP-Gly domain comprises at least 5 contiguous amino acid residues derived from the CAP-Gly domain.

3. The polypeptide of claim 2, wherein the contiguous amino acid residues are those described by a portion of SEQ ID NO: 1.

4. The polypeptide of claim 2, wherein the polypeptide is isolated.

5. The polypeptide of claim 2, wherein the polypeptide is purified.

6. The polypeptide of claim 1, wherein the polypeptide has a CAP-Gly domain with structure analogous to the structure of the CAP-Gly domain of F53F4.3 protein.

7. The polypeptide of claim 6, wherein the structure of the CAP-Gly domain of the polypeptide is substantially the same as the structure of the CAP-Gly domain of F53F4.3 protein.

8. An isolated polypeptide comprising an amino acid sequence according to amino acid residues 135 to 229 of a cytoskeletal-associated protein with structure analogous to the structure of the CAP-Gly domain of F53F4.3 protein.

9. The polypeptide of claim 8, wherein the structure of the CAP-Gly domain of the polypeptide is substantially the same as the structure of the CAP-gly domain of F53F4.3 protein.

10. The polypeptide of claim 8, wherein a portion of the amino acid sequence is GKNDG (SEQ ID NO: 2).

11. The polypeptide of claim 8, wherein a portion of the amino acid sequence is GKHDG (SEQ ID NO: 3).

12. The polypeptide of claim 8, wherein a portion of the amino acid sequence is GKNSG (SEQ ID NO: 4).

13. The polypeptide of claim 8, wherein a portion of the amino acid sequence is GKHSG (SEQ ID NO: 5).

14. A crystal of the CAP-Gly domain, wherein the space group of the crystal is P6₁22 and unit cell dimensions of the crystal are about a=64±3 Å, b=64±3 Å, and c=102±3 Å.

15. A crystal of the CAP-Gly domain, wherein the space group of the crystal is P6₁22 and unit cell dimensions are about a=64±2 Å, b=64±2 Å, and c=102±2Å.

16. A crystal of the CAP-Gly domain, wherein the space group of the crystal is P6₁22 and unit cell dimensions are about a=64±1 Å, b=64±1 Å, and c=102±1 Å.

17. The crystal of the CAP-Gly domain of claim 14, wherein the CAP-Gly domain has a three-dimensional structure characterized by the atomic structure coordinates of Table 2.

18. The crystal of the CAP-Gly domain of claim 14, wherein the crystal is formed from a polypeptide comprising amino acid sequence of at least 5 contiguous amino acid residues derived from the CAP-Gly domain.

19. A method of characterizing protein structures comprising the steps:
(a) determining the three-dimensional structure of the CAP-Gly domain;
(b) determining the three-dimensional structure of an experimental protein;
(c) comparing the three-dimensional structure of the experimental protein to the three-dimensional structure of the CAP-Gly domain; and
(d) recording variances between the three-dimensional structure of the CAP-Gly domain and the experimental protein.

20. The method of claim 19, wherein the three-dimensional structure of the CAP-Gly domain is derived from the structure of a polypeptide comprising amino acid sequence of at least 5 contiguous amino acid residues derived from the CAP-Gly domain.

21. The method of claim 19 wherein the three-dimensional structure of the CAP-Gly domain is derived from a crystal of the CAP-Gly domain, wherein the space group of the crystal is P6122 and unit cell dimensions of the crystal are about a=64±3 Å, b=64±3 Å, and c=102±3 Å.

22. The method of claim 21, wherein the three-dimensional structure of the CAP-Gly domain is defined by the atomic structure coordinates of Table 2.

23. A method of evaluating two or more experimental proteins in respect to the CAP-Gly domain, comprising:
(a) evaluating the variances of (d) of claim 19 for a first experimental protein;
(b) evaluating the variances of (d) of claim 19 for a second experimental protein; and
(c) ranking the experimental protein with the least variance from the structure of CAP-Gly domain as being most similar.

24. A method for generating analogs of polypeptides comprising the CAP-Gly domain, comprising:
(a) determining the structure of a CAP-Gly domain;
(b) selecting a polypeptide comprising an amino acid sequence that maintains a CAP-Gly domain structure; and
(c) generating an analog polypeptide comprising the amino acid sequence according to step (b) that maintains the CAP-Gly domain structure.

25. A method for determining whether an analog of the CAP-Gly domain will have an altered three-dimensional structure as compared to the CAP-Gly domain, comprising:
(a) determining the three-dimensional coordinates of atoms of a CAP-Gly domain;
(b) providing a computer having a memory means, a data input means, a visual display means, the memory means containing three-dimensional molecular simulation software operable to retrieve coordinate data from the memory means and to display a three-dimensional representation of a molecule on the visual display means and being operable to produce a three-dimensional representation of an analog of the molecule responsive to operator-selected changes to the chemical structure of the molecule and to display the three-dimensional representation of the analog;
(c) inputting three-dimensional coordinate data of the atoms of the CAP-Gly domain into the computer and storing the data in the memory means;
(d) displaying a three-dimensional representation of the CAP-Gly domain on the visual display means;
(e) inputting into the data input means of the computer at least one operator-selected change in chemical structure of the CAP-Gly domain;
(f) executing the molecular simulation software to produce a modified three-dimensional molecular representation of the analog structure; and
(g) displaying the three-dimensional representation of the analog on the visual display means, whereby changes in three-dimensional structure of the Cap-Gly domain consequent on changes in chemical structure can be visually determined.

26. The method according to claim 25, wherein the determination of the analog structure comprises displaying on the visual display means the three-dimensional structure of both the original CAP-Gly domain and the CAP-Gly domain analog, visually comparing the configuration and spatial arrangement of the CAP-Gly domain, and selecting an analog structure wherein the domains are substantially the same.

27. A method for identifying CAP-Gly domain analogs that mimic the three-dimensional structure of the CAP-Gly domain, comprising:
(a) producing a multiplicity of analog structures of the CAP-Gly domain by the method of claim 25, and
(b) selecting an analog structure represented by a three-dimensional representation wherein the three-dimensional configuration and spatial arrangement of regions involved in function of the CAP-Gly domain remain substantially preserved.

28. A method for producing an analog of a CAP-Gly domain that mimics the three-dimensional structure of the CAP-Gly domain, comprising:
(a) determining the three-dimensional coordinates of atoms of an CAP-Gly domain;
(b) providing a computer having a memory means, a data input means, a visual display means, the memory means containing three-dimensional molecular simulation software operable to retrieve coordinate data from the memory means and to display a three-dimensional representation of a domain on the visual display means and being operable to produce a modified three-dimensional analog representation responsive to operator-selected changes to the chemical structure of the domain and to display the three-dimensional representation of the modified analog;
(c) inputting three-dimensional co-ordinate data of atoms of the CAP-Gly domain into the computer and storing the data in the memory means;
(d) inputting into the data input means of the computer at least one operator-selected change in chemical structure of the CAP-Gly domain;
(e) executing the molecular simulation software to produce a modified three-dimensional molecular representation of the analog structure;
(f) displaying the three-dimensional representation of the analog on the visual display means, whereby changes in three-dimensional structure of the CAP-Gly domain consequent on changes in chemical structure can be visually monitored;
(g) repeating steps (d) through (f) to produce a multiplicity of analogs;
(h) selecting an analog structure represented by a three-dimensional representation wherein the three-dimensional configuration and spatial arrangement of regions involved in function of the CAP-Gly domain remain substantially preserved;
(i) synthesizing the selected analog by means of recombinant DNA technology; and
(j) determining the CAP-Gly domain function of the synthesized CAP-Gly domain analog, whereby an analog having the activity is a mimic of the three-dimensional structure of the CAP-Gly domain.

29. A method for identifying a potential ligand of a CAP-Gly domain containing protein, comprising:
(a) using a three-dimensional structure of the CAP-Gly domain or portions thereof as defined by atomic coordinates of F53F4.3 according to Table 2;
(b) employing the three-dimensional structure to design or select the potential ligand;
(c) synthesizing the potential ligand;
(d) contacting the potential ligand with the CAP-Gly domain containing protein; and
(e) determining whether the potential ligand binds to the CAP-Gly domain containing protein.

30. The method according to claim 29, wherein the step of employing the three-dimensional structure to design or select the ligand comprises:
(a) identifying chemical functionalities capable of associating with the CAP-Gly domain; and
(b) assembling the identified chemical functionalities into a single molecule to provide the structure of the CAP-Gly domain potential ligand.

31. The method according to claim 30, wherein the potential ligand is designed de novo.

32. The method according to claim 30, wherein the potential ligand is designed from a known compound.

33. The method of claim 29, wherein the CAP-Gly domain of (a) consists essentially of sequence corresponding to amino acid residue 135 through amino acid residue 229 of F53F4.3 from Candida elegans.

34. The method of claim 29, wherein the set of atomic coordinates obtained in step (a) are obtained using a crystal having the space group of P6₁22 and unit cell dimensions of about a=64±3 Å, b=64±3 Å, and c=102±3 Å.

35. The method of claim 34, wherein the atomic coordinates are the atomic coordinates in Table 2.

36. An analog of the CAP-Gly domain made by;
(a) determining the structure of a CAP-Gly domain;
(b) selecting a polypeptide comprising an amino acid sequence that maintains a CAP-Gly domain structure; and
(c) generating an analog polypeptide comprising the amino acid sequence according to step (b) that maintains the CAP-Gly domain structure.

37. An analog of the CAP-Gly domain made by;
(a) determining the three-dimensional coordinates of atoms of an CAP-Gly domain;
(b) providing a computer having a memory means, a data input means, a visual display means, the memory means containing three-dimensional molecular simulation software operable to retrieve coordinate data from the memory means and to display a three-dimensional representation of a domain on the visual display means and being operable to produce a modified three-dimensional analog representation responsive to operator-selected changes to the chemical structure of the domain and to display the three-dimensional representation of the modified analog;
(c) inputting three-dimensional co-ordinate data of atoms of the CAP-Gly domain into the computer and storing the data in the memory means;
(d) inputting into the data input means of the computer at least one operator-selected change in chemical structure of the CAP-Gly domain;
(e) executing the molecular simulation software to produce a modified three-dimensional molecular representation of the analog structure;
(f) displaying the three-dimensional representation of the analog on the visual display means, whereby changes in three-dimensional structure of the CAP-Gly domain consequent on changes in chemical structure can be visually monitored;
(g) repeating steps (d) through (f) to produce a multiplicity of analogs;
(h) selecting an analog structure represented by a three-dimensional representation wherein the three-dimensional configuration and spatial arrangement of regions involved in function of the CAP-Gly domain remain substantially preserved;
(i) synthesizing the selected analog by means of recombinant DNA technology; and
(j) determining the CAP-Gly domain function of the synthesized CAP-Gly domain analog, whereby an analog having the activity is a mimic of the three-dimensional structure of the CAP-Gly domain.

38. An analog structure of a CAP-Gly domain produced by;
(a) determining the three-dimensional coordinates of atoms of a CAP-Gly domain;
(b) providing a computer having a memory means, a data input means, a visual display means, the memory means containing three-dimensional molecular simulation software operable to retrieve coordinate data from the memory means and to display a three-dimensional representation of a molecule on the visual display means and being operable to produce a three-dimensional representation of an analog of the molecule responsive to operator-selected changes to the chemical structure of the molecule and to display the three-dimensional representation of the analog;
(c) inputting three-dimensional coordinate data of the atoms of the CAP-Gly domain into the computer and storing the data in the memory means;
(d) displaying a three-dimensional representation of the CAP-Gly domain on the visual display means;
(e) inputting into the data input means of the computer at least one operator-selected change in chemical structure of the CAP-Gly domain;
(f) executing the molecular simulation software to produce a modified three-dimensional molecular representation of the analog structure; and
(g) displaying the three-dimensional representation of the analog on the visual display means, whereby changes in three-dimensional structure of the Cap-Gly domain consequent on changes in chemical structure can be visually determined.

39. The analog structure of a CAP-Gly domain of claim 38, wherein the production of the analog structure comprises determination of whether the analog structure is altered comprises displaying on the visual display means the three-dimensional structure of both the original CAP-Gly domain and the CAP-Gly domain analog, visually comparing the configuration and spatial arrangement of the CAP-Gly domain, and selecting an analog structure wherein the domains are substantially the same.

40. A ligand of CAP-Gly domain containing polypeptide, wherein method of identifying the ligand comprises:
(a) using a three-dimensional structure of any CAP-Gly domain or portions thereof as defined by atomic coordinates of F53F4.3 according to Table 2;
(b) employing the three-dimensional structure to design or select the potential ligand;
(c) synthesizing the potential ligand;
(d) contacting the potential ligand with the CAP-Gly domain containing protein; and
(e) determining whether the potential ligand binds to the CAP-Gly domain containing protein.

41. A method for identifying an interacting partner for a protein containing a CAP-Gly domain, comprising:
(a) providing a CAP-Gly domain or analog thereof;
(b) contacting the CAP-Gly domain or analog thereof with potential. interacting partners; and
(c) determining the presence of interaction between the CAP-Gly domain or analog thereof and the potential interacting partners, thereby identifying an interacting partner of the protein containing a CAP-Gly domain.

42. The method of claim 41, wherein the CAP-Gly domain or analog thereof of (a) is a polypeptide, CAP-Gly domain or analog that mimics structure of a portion of the CAP-Gly domain.

43. The method of claim 41, wherein the CAP-Gly domain or analog thereof of (a) is made by a method for generating analogs of polypeptides comprising the CAP-Gly domain, comprising:
(a) determining the structure of a CAP-Gly domain;
(b) selecting a polypeptide comprising an amino acid sequence that maintains a CAP-Gly domain structure; and
(c) generating an analog polypeptide comprising the amino acid sequence according to step (b) that maintains the CAP-Gly domain structure.

44. The method of claim 41, wherein the CAP-Gly domain or analog thereof of (a) is made by a method for generating analogs, comprising:
(a) determining the three-dimensional coordinates of atoms of an CAP-Gly domain;
(b) providing a computer having a memory means, a data input means, a visual display means, the memory means containing three-dimensional molecular simulation software operable to retrieve coordinate data from the memory means and to display a three-dimensional representation of a domain on the visual display means and being operable to produce a modified three-dimensional analog representation responsive to operator-selected changes to the chemical structure of the domain and to display the three-dimensional representation of the modified analog;
(c) inputting three-dimensional co-ordinate data of atoms of the CAP-Gly domain into the computer and storing the data in the memory means;
(d) inputting into the data input means of the computer at least one operator-selected change in chemical structure of the CAP-Gly domain;
(e) executing the molecular simulation software to produce a modified three-dimensional molecular representation of the analog structure;
(f) displaying the three-dimensional representation of the analog on the visual display means, whereby changes in three-dimensional structure of the CAP-Gly domain consequent on changes in chemical structure can be visually monitored;
(g) repeating steps (d) through (f) to produce a multiplicity of analogs;
(h) selecting an analog structure represented by a three-dimensional representation wherein the three-dimensional configuration and spatial arrangement of regions involved in function of the CAP-Gly domain remain substantially preserved;
(i) synthesizing the selected analog by means of recombinant DNA technology; and
(j) determining the CAP-Gly domain function of the synthesized CAP-Gly domain analog, whereby an analog having the activity is a mimic of the three-dimensional structure of the CAP-Gly domain.

45. An apparatus for determining whether a compound will interact with a protein containing a CAP-Gly domain, comprising:
(a) a memory that stores
(i) the three-dimensional coordinates and identities of the atoms of the CAP-Gly domain that together form a solvent-accessible surface; and
(ii) executable instructions; and
(b) a processor that executes instructions to:
(i) receive three-dimensional structural information for a candidate compound;
(ii) determine if the three-dimensional structure of the candidate compound is complementary to the structure of the solvent-accessible surface of the CAP-Gly domain; and
(iii) output the results of the determination.

46. The apparatus of claim 45, wherein the three-dimensional coordinates and identities of atoms of the CAP-Gly domain are derived from the structure of amino acid residue 135 through amino acid residue 229 of F53F4.3 from Candida elegans.

47. The apparatus of claim 46, wherein the set of three-dimensional coordinates and identities of atoms of the CAP-Gly domain are derived from a crystal having the space group of P6₁22 and unit cell dimensions of approximately a=b=64 Å and c=102 Å.

48. The apparatus of claim 46, wherein the three-dimensional coordinates and identities of atoms of the CAP-Gly domain are the atomic coordinates in Table 2.

49. A computer-readable storage medium comprising digitally-encoded structural data, wherein the data comprise the identity and three-dimensional coordinates of at least 6 amino acids of the CAP-Gly domain.

50. The medium of claim 49, wherein the data comprise the identity and three-dimensional coordinates of at least 8 amino acids of the CAP-Gly domain.

51. The medium of claim 49, wherein the data comprise the identity and three-dimensional coordinates of at least 10 amino acids of the CAP-Gly domain.

52. The medium of claim 49, wherein the data comprise the identity and three-dimensional coordinates of at least 15 amino acids of the CAP-Gly domain.

53. The medium of claim 49, wherein the data comprise the identity and three-dimensional coordinates of at least 20 amino acids of the CAP-Gly domain.

54. The computer-readable storage medium of claim 49, wherein the data comprises the atomic coordinates in Table 2 or a portion thereof.

55. A repository of reference three-dimensional coordinates, and software configured to:
(a) receive a subject set of coordinates which comprise a subject structure;
(b) compare each subject set of coordinates to the reference set of coordinates;
(c) calculate the root mean squared deviation of the subject set of coordinates from the reference set of coordinates; and
(d) compare the root mean squared deviation from step (c) to limit values, whereby if the deviation from step (c) is less than or equal to the limit values, the subject structure is assigned a function based on the subject structure's similarity to CAP-Gly domain structure.

56. The repository and software of claim 55, wherein the reference set of coordinates are those coordinates in Table 2 or a portion thereof.

57. The repository and software of claim 55, wherein the limit values of (d) correspond to values less than or equal to 3 Å in root mean squares deviation.

58. The repository and software of claim 55, wherein the limit values correspond to values less than or equal to 2.5 Å in root mean squares deviation.

59. The repository and software of claim 55, wherein the limit values correspond to values less than or equal to 2 Å in root mean squares deviation.

60. The repository and software of claim 55, wherein the limit values correspond to values less than or equal to 1.5 Å in root mean squares deviation.

61. The repository and software of claim 55, wherein the limit values correspond to values less than or equal to 1 Å in root mean squares deviation.

62. The repository and software of claim 55, wherein the limit values correspond to values less than or equal to 0.5 Å in root mean squares deviation.

63. The repository and software of claim 55, wherein the limit values correspond to values less than or equal to 0.2 Å in root mean squares deviation.

64. The repository and software of claim 55, wherein the limit values correspond to values less than or equal to 0.1 Å in root mean squares deviation.

65. A method of determining relationships between two or more polypeptide structures, comprising:
(a) obtaining a reference structure, wherein the reference structure is a structure of a polypeptide comprising the CAP-Gly domain or a portion thereof;
(b) obtaining at least one subject structure;
(c) determining a topology diagram for each of the reference and subject structures;
(d) comparing the topology diagram of the reference structure and the topology diagram of the subject structure; and
(e) assigning a relationship between the reference structure and any subject structure, wherein if the topology diagrams of the subject structures correspond to the topology diagram of the reference structure, the proteins have substantially the same protein fold.

66. The method of claim 65, wherein the reference structure is a structure defined by the atomic coordinates of Table 2.

67. The method of claim 65, wherein determination of the topology diagram considers secondary structural elements, spatial adjacency within fold and approximate orientation.

68. The method of claim 67, wherein determination of the topology diagram neglects the length of loop elements.

69. The method of claim 68, wherein determination of the topology diagram neglects the structure of loop elements.

70. The method of claim 69, wherein the topology diagram neglects spatial orientations of secondary structural elements.

71. The method of claim 65, wherein the topology diagrams are determined using TOPS protein topology search, pattern discovery and structure comparison.

72. A polypeptide that comprises any amino acid sequence that adopts structure substantially similar to that of a polypeptide comprising the CAP-Gly domain or a portion thereof as indicated by a method of comparison, comprising:
(a) obtaining a reference structure, wherein the reference structure is a structure of a polypeptide comprising the CAP-Gly domain or a portion thereof;
(b) obtaining at least one subject structure;
(c) determining a topology diagram for each of the reference and subject structures;
(d) comparing the topology diagram of the reference structure and the topology diagram of the subject structure; and
(e) assigning a relationship between the reference structure and any subject structure, wherein if the topology diagrams of the subject structures correspond to the topology diagram of the reference structure, the proteins have substantially the same protein fold.

73. The polypeptide of claim 72, wherein the amino acid sequence comprises greater than 5 contiguous amino acid residues.

74. The polypeptide of claim 72, wherein the amino acid sequence comprises greater than 7 contiguous amino acid residues.

75. The polypeptide of claim 72, wherein the amino acid sequence comprises greater than 9 contiguous amino acid residues.

76. The polypeptide of claim 72, comprising more than one amino acid sequence that adopts structure substantially similar to that of a polypeptide comprising the CAP-Gly domain or a portion thereof.

77. The polypeptide of claim 76, wherein the CAP-Gly domain structure is the structure defined by atomic coordinates from Table 2.

78. A method of identifying a compound that alters a function of a CAP-Gly domain containing protein comprising:
(a) providing a model of the structure of the CAP-Gly domain;
(b) studying the interaction of at least one candidate ligand with the model;
(c) selecting a compound which is predicted to act as a ligand; and
(d) determining that the selected compound will alter a function of a CAP-Gly domain containing protein.

79. The method of claim 78, wherein (a) comprises use of atomic coordinate data according to Table 2.

80. The method of claim 78, wherein (b) comprises studying the interaction of a ligand with amino acid residues selected from the group consisting of sequence according to Gly189 to Gly193, of sequence according to Val156 to Met106, Arg162, Tyr168, Phe174, Trp179, Lys190, Asn191, Val195, Tyr200, Phe201, Gly209, Phe210, and Val211 of F54F4.3, homologs, and conservative variations thereof.

81. The method of claim 78, wherein (b) comprises studying the interaction of a ligand with amino acid residues selected from the group consisting of sequence according to Gly189 to Gly193, of sequence according to Val156 to Met160, Arg162, Tyr168, Phe174, Trp179, Lys190, Asn191, Val195, Tyr200, Phe201, Gly209, Phe210, and Val211 of F54F4.3 and homologs thereof.

82. The method of claim 78, wherein (c) comprises use of molecular dynamics calculations.

83. The method of claim 78, wherein (c) comprises visual inspection of the provided model of the structure of the CAP-Gly domain and the compound.

84. The method of claim 78, wherein (c) comprises use of assays to determine binding or absence of binding between the compound and a CAP-Gly domain.

85. The method of claim 84, wherein (d) comprises use of an in vivo assay.

86. The method of claim 84, wherein (d) comprises use of an in vitro assay.

87. The method of claim 84, wherein (d) comprises use of a virus assembly assay.

88. The method of claim 87, wherein the virus assembly assay monitors the assembly of a virus selected from the group consisting of large DNA viruses and recombinants and variants thereof.

89. The method of claim 88, wherein the large DNA viruses can be selected from the group consisting of poxviruses, iridoviruses, and African swine fever virus.

90. The method of claim 84, wherein (d) comprises use of an assay that monitors chaperone activity.

91. A method of screening compounds to identify ligands with biological effects, the method comprising:
(a) contacting a polypeptide comprising a CAP-Gly domain with at least one compound;
(b) assaying for a selected biological effect;
(c) assaying for the selected biological effect in the absence of the at least one compound; and
(d) comparing the level of the selected biological effect in (b) to that in (c), whereby compounds are identified as ligands with biological effects when the level of the selected biological effect in (b) differs from the level of the selected biological effect in step (c).

92. The method of claim 91, wherein the compounds are selected from a chemical library.

93. The method of claim 91, wherein the compounds are selected from a natural products library.

94. The method of claim 91, wherein the compounds are selected from a combinatorial library.

95. The method of claim 91, wherein the biological effect is selected from the group consisting of microtubule formation, microtubule organization, viral capsid formation, virus factory formation, plaque formation, aggresome formation and chaperone activity.

96. The method of claim 91, wherein the method is an in vivo assay.

97. The method of claim 91, wherein the method is an ex vivo assay.

98. The method of claim 91, wherein the method is an in vitro assay.
